(19) United States
(12) Patent Application Publication
(10) Pub. No.: US 2026/0226489 A1
(43) Pub. Date: Aug. 6, 2026

(54) DWARF PLANT VARIETIES THROUGH MUTATION OF ETTIN GENE UPSTREAM OPEN READING FRAMES

(71) Applicant: genXtraits, Inc., Alameda, CA (US)

(72) Inventors: Roger P. Hellens, Ngongotaha (NZ); Oliver J. Ratcliffe, Alameda, CA (US); Jeffrey M. Libby, Cupertino, CA (US)

(21) Appl. No.: 19/148,936

(22) PCT Filed: Feb. 3, 2024
(86) PCT No.: PCT/US2024/014365

§ 371 Date: Jul. 17, 2025

Related U.S. Application Data
(60) Provisional application No. 63/443,444, filed on Feb. 5, 2023.

(51) Int. Cl. C12N 15/82; C07K 14/415; C12N 9/22; C12N 15/11

(52) U.S. Cl. CPC C12N 15/8262; C07K 14/415; C12N 9/226; C12N 15/11; C12N 15/8213; C12N 2310/20

(57) ABSTRACT

Crop and other plants with increasing the expression of the transcription factor ETTIN are disclosed. The expression of ETTIN in a plant is increased by reducing the transcription of an Upstream OPEN READING FRAME (uORF) which is located upstream of the ETTIN coding sequence. Reduced expression of the ETTIN gene confers a dwarf phenotype in the plant. The plant in which the ETTIN uORF is targeted may be a rootstock of a crop plant. The rootstock may be grafted to a scion of a crop plant and the scion exhibits a dwarf phenotype. The present disclosure provides a means of creating mutations in uORFs DNA repressor elements for the purpose of generating new dwarf crop varieties.

### FIELD OF THE INVENTION

This disclosure relates to compositions and methods for the development of plant varieties.

### BACKGROUND OF THE INVENTION

The ETTIN or Auxin Response Factor 3 (ARF3) gene and related paralogs from the ARF family each encodes proteins that regulates a plant's response to auxin. Generally, ETTIN (SEQ ID NO: 1, 2) promotes a short bushy growth habit in a wide variety of plants. An ETTIN gene with altered expression or activity may result in elevated levels of ETTIN protein which produces a plant with reduced apical dominance, a bushier habit, an altered xylem/phloem ratio, an increased number of phloem elements, reduced root mass, and/or other dwarfing-associated phenotypes. A plant comprising such an ETTIN gene with altered expression or activity may be suitable for use as a rootstock plant, particularly since it has been shown that a scion grafted to a rootstock with increased ETTIN expression exhibits dwarf phenotype characteristics (patent publication US20180371481, 2018, Foster).

Auxin Response Factors (ARFs) are a class of transcription factor (of which ETTIN is a member) which activate regulate the transcription of target genes in response to changes in the level of auxin. ARFs interact with AUX/IAA proteins, which have a repressive role and result in ARFs being degraded (Reed (2001) Trends Plant Sci. 6, 420-425.) Thus, the activity of an ARF protein can be increased by removing its capacity to interact with AUX/IAA proteins. This can be achieved, amongst other means, by introducing mutations which compromise residues in the ARF domain that interacts with AUX/IAA proteins.

Generally, Upstream Open Reading Frames (uORFs), which reside in the 5′ untranslated region of an mRNA, are DNA repressor elements that repress the downstream coding sequence (CDS; Calvo et al, 2009. Proc. Natl. Acad. Sci. USA 106:7507-7512; Arribere & Gilbert, 2013. Genome Res 23:977-987). Generally, uORFs regulate eukaryotic gene expression and their translation usually inhibits downstream expression of the primary ORF.

Since expression of ETTIN promotes a dwarf phenotype, it follows that increasing the expression of ETTIN in a plant by targeting and reducing the activity of a uORF upstream of the ETTIN CDS will confer a dwarf phenotype in a plant. The instant disclosure is thus directed to the generation of mutations in an ETTIN uORF that releases the repression of translation of the ETTIN gene, which results in elevated levels of the polypeptide and a more extreme short, bushy growth habit. Alternatively, in instances where a non-bushy architecture (i.e. increased apical dominance) is desired, such as in trees grown for timber, mutations that strengthen a uORF and further increase the repression of an ETTIN homolog, could be desirable.

The plant in which the ETTIN uORF is targeted may be a rootstock of a crop plant. The rootstock may be grafted to a scion of a crop plant and generate a developmental signal causing the scion to exhibit a dwarf phenotype. If an ETTIN uORF is strengthened, for example by converting its non-canonical start codon to ATG, a rootstock could be produced that cause the grafted scion to grow with a long straight main stem and minimal branching.

The present disclosure provides a means of creating mutations in uORFs DNA repressor elements for the purpose of generating new dwarf crop varieties.

### SUMMARY

The present disclosure is directed to approaches that disrupt the function of uORFs in the 5′UTRs of ETTIN genes by creating or selecting mutations therein. A favored approach involves introducing gene editing construct or constructs into a plant cell, for example, a CRISPR/Cas construct, that comprises a polynucleotide sequence that encodes a Cas enzyme (e.g., Cas9), and at least one guide RNA sequence with complementarity to a uORF sequence. The guide RNA sequence targets a target site in a uORF nucleotide sequence that encodes a uORF polypeptide in a plant. The present description provides for uORF polypeptides that are at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a predicted protein sequence encoded by a uORF (uPEP) located upstream of a nucleotide sequence that encodes any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, or a polypeptide encoded by any of SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240.

In the present disclosure, the gene editing construct interacts with a target site that encodes a uORF polypeptide and edits the target site that encodes a uORF polypeptide which alters the expression level or activity of an ETTIN polypeptide in the plant cell. The plant cell is then regenerated into a plant with an elevated level or activity of an ETTIN polypeptide. As a result, the plant exhibits at least one dwarfing-associated attribute and/or a dwarf phenotype. Optionally, the resulting plant can be vegetatively, selfed, and/or crossed to a second plant. The resulting progeny plants then exhibit a dwarf phenotype.

The present disclosure is also directed to a plant, plant part, or plant cell, the genome of which harbors a non-naturally occurring allele of a gene that encodes an ETTIN homolog polypeptide, or a uORF polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a uPEP encoded by a uORF located upstream of a nucleotide sequence that encodes any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, or a polypeptide encoded by any of SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240, sequences of ETTIN proteins from different crops. The non-naturally occurring allele comprises a mutation in a uORF within the 5′UTR of the gene that encodes the polypeptide. The allele in question may be produced by a directed technique such as gene editing or by selecting a mutant plant carrying the allele from a mutagenized population (such as a TILLING population), with the selection being made based on either the presence of a dwarf phenotype, and elevated level of the polypeptide, or by DNA sequencing to identify mutations in the UTR, as compared to a wild-type plant.

The plant part carrying the above non-naturally occurring allele may be a root stock that is grafted to a scion to produce a plant that exhibits a dwarf phenotype when grown to maturity. A plant exhibiting a dwarf phenotype may then be selected by identifying an attribute associated with a dwarf phenotype, or by identifying a rootstock with competence to induce an attribute associated with a dwarf phenotype.

A mutation in the plant or plant part is introduced into a uORF is at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or about 100% to a sequence from the SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240.

The present disclosure also pertains to a method of producing new plant variety comprising introducing or selecting a mutation in a cell of the plant species. The mutation is in a uORF sequence within the 5′UTR of gene that encodes a polypeptide that is an ETTIN homolog or a polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a uPEP encoded by a uORF located upstream of a nucleotide sequence that encodes any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, or a polypeptide encoded by any of SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240. The cell is then regenerated into a plant or plant part which is selected for an increased level of the polypeptide and the presence of a dwarf phenotype, and then multiplying the selected plant or plant part through a process of vegetative propagation, grafting or tissue culture or crossing.

### BRIEF DESCRIPTION OF THE SEQUENCE LISTING

The Sequence Listing provides exemplary polynucleotide and polypeptide sequences of the invention. Traits associated with the use of the sequences are included in the Examples.

The sequence listing provided in the file entitled “GXTR-0003PCT, which is an XML text file that was created on 1 Feb. 29, 2024 submitted with this application, and which comprises 264,890 bytes, is hereby incorporated by reference in its entirety.

### DETAILED DESCRIPTION

The present description is directed to compositions of plants with a dwarf phenotype and methods for producing plants with a dwarf phenotype.

The present description relates to polynucleotides and polypeptides, for example, for modifying phenotypes of plants. Throughout this disclosure, various information sources are referred to and/or are specifically incorporated. The information sources include scientific journal articles, patent documents, textbooks, and World Wide Web browser-inactive page addresses, for example. The contents and teachings of the information sources can be relied on and used to make and use embodiments of the invention.

As used herein and in the appended claims, the singular forms “a,” “an,” and “the” include plural reference unless the context clearly dictates otherwise. Thus, for example, a reference to “a plant” includes a plurality of such plants, and, for example, a reference to “a stress” is a reference to one or more stresses and equivalents thereof known to those skilled in the art, and so forth.

### Definitions

“Identity” or “similarity” refers to sequence similarity between two polynucleotide sequences or between two polypeptide sequences, with identity being a stricter comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching, or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

The term “variant”, as used herein, may refer to polynucleotides or polypeptides that differ from the presently disclosed polynucleotides or polypeptides, respectively, in sequence from each other, and as set forth below.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences o may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Non-naturally occurring allele” means an allele of a gene that has been produced or selected through human intervention including through application of gene editing, or selection of plants or plant cells harboring a desired sequence change from among a larger population of mutated plants or plant cells with an introduced mutation (that is, a mutation introduced by synthetic means).

The term “plant” includes whole plants, shoot vegetative organs/structures (e.g., leaves, stems, and tubers), roots, flowers, and floral organs/structures (e.g., bracts, sepals, petals, stamens, carpels, anthers, and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae. (See for example, from Daly et al. (2001) Plant Physiol. 127:1328-1333 Ku et al. (2000) Proc. Natl. Acad. Sci. 97:9121-9126; and see also Tudge, in The Variety of Life, Oxford University Press, New York, NY (2000) pp. 547-606).

In the context of a plant, “dwarf” refers to an individual or variety which exhibits, as compared to wild-type plant of the same species, a phenotype that comprises a shorter or bushier stature, reduced apical dominance, increased outgrowth of secondary or higher order shoots, increased branching, reduced height, shorter internodes, and/or flowering branches which exhibit a reduced number of vegetative nodes prior to the production of floral nodes.

A “control plant” as used in the present invention refers to a plant cell, seed, plant component, plant tissue, plant organ or whole plant used to compare against a mutant, gene-edited, transgenic, or genetically modified plant for the purpose of identifying an enhanced phenotype in the mutant, gene-edited, transgenic, or genetically modified plant. A control plant may in some cases be a transgenic plant line that comprises an empty vector or marker gene, but does not contain a recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic wild-type plant of the parental line used to generate a gene edited, mutant, or transgenic plant herein.

“Homology” refers to sequence similarity between a reference sequence and at least a fragment of a sequence of interest or a newly sequenced clone insert or its encoded amino acid sequence.

Homologous protein sequences are those with a common evolutionary origin. There are two types of protein homologs, depending on how they originated: paralogs, derived from a gene duplication event, and orthologs, originated from a speciation event. The ortholog conjecture postulates that orthologous sequences are functionally more similar than paralogous sequences in comparable divergence times (Mier, Pérez-Pulido and Andrade-Navarro. BMC Bioinformatics 19, 431 (2018). doi.org/10.1186/s12859-018-2457-y).

“ETTIN homolog” means a protein, which upon performing a BLAST analysis against the set of proteins encoded by the Arabidopsis proteome, returns a higher level of sequence identity to the Arabidopsis ETTIN protein or a paralog of ETTIN than to any other protein in the Arabidopsis proteome, and which has a level of similarity to the Arabidopsis ETTIN protein equal or higher than an HSP of bit score 50.

“Arabidopsis ETTIN protein” means a product of the ETTIN gene which corresponds to the locus identified by Arabidopsis genome identifier AT2G33860 and which is also known by the alternative names ARF3, AUXIN RESPONSE TRANSCRIPTION FACTOR 3, and ETT.

“uPEP” or “uPeptide” as used herein refers to the peptide which is encoded by a uORF.

In general, the term “variant” refers to molecules with some differences, generated synthetically or naturally, in their base or amino acid sequences as compared to a reference (native) polynucleotide or polypeptide, respectively. These differences include substitutions, insertions, deletions, or any desired combinations of such changes in a native polynucleotide of amino acid sequence.

A “conserved domain”, with respect to presently disclosed polypeptides refers to a domain within a polypeptide factor family that exhibits a higher degree of sequence homology, such as at least at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 96%, at least 97%, at least 98%, at least 99% or about 100% amino acid residue sequence identity of a polypeptide of consecutive amino acid residues. A fragment or domain can be referred to as outside a conserved domain, outside a consensus sequence, or outside a consensus DNA-binding site that is known to exist or that exists for a particular protein class, family, or sub-family. In this case, the fragment or domain will not include the exact amino acids of a consensus sequence or consensus DNA-binding site of a polypeptide class, family or sub-family, or the exact amino acids of a particular protein consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence. Thus, by using alignment methods well known in the art, the conserved domains of the plant proteins for Auxin Response Factor 3 protein homologs, orthologs, or paralogs.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance. A trait can also include an increased level of a polypeptide, which may be detected by Western blotting, antibody-based techniques, or mass spectrometry. A variety of analytical techniques can be used to measure the amount of, comparative level of, or difference in any selected chemical compound or macromolecule in artificially modified plants, however.

“Trait modification” refers to producing a detectable difference in a characteristic in a mutant or gene edited plant ectopically expressing or containing an elevated level of a polynucleotide or polypeptide of the present invention relative to a plant not doing so, such as a wild-type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, or at least about a 100%, or an even greater difference compared with a wild-type plant. It is known that there can be a natural variation in the modified trait. Therefore, the trait modification observed entails a change of the normal distribution of the trait in the plants compared with the distribution observed in wild-type plant.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs, or whole plants may be used as controls to compare levels of expression and the extent and nature of trait modification with cells, tissue, or plants of the same species in which a polypeptide's expression is altered, e.g., in that it has been knocked out, elevated overexpressed, or ectopically expressed.

“TILLING” is an acronym for a technique that involves “Targeting Induced Local Lesions in Genomes” whereby a plant breeder creates a mutant population through means of mutagens (such as EMS, X-rays, T-DNAs or transposon) and then individual plants carrying mutations in a target DNA sequence of interest are selected by sequencing that target region from a large number of plants and choosing the individuals that have changes in that target sequence compared to the same sequence from a wild-type plant.

“Yield” or “plant yield” refers to increased plant growth, increased crop growth, increased biomass, and/or increased plant product production, and is dependent to some extent on temperature, plant size, organ size, planting density, light, water, and nutrient availability, and how the plant copes with various stresses, such as through temperature acclimation and water or nutrient use efficiency.

“Planting density” refers to the number of plants that can be grown per acre. For crop species, planting or population density varies from a crop to a crop, from one growing region to another, and from year to year. Using corn as an example, the average prevailing density in 2000 was in the range of 20,000-25,000 plants per acre in Missouri, USA. A desirable higher population density (a measure of yield) would be at least 22,000 plants per acre, and a more desirable higher population density would be at least 28,000 plants per acre, more preferably at least 34,000 plants per acre, and most preferably at least 40,000 plants per acre. The average prevailing densities per acre of a few other examples of crop plants in the USA in the year 2000 were: wheat 1,000,000-1,500,000; rice 650,000-900,000; soybean 150,000-200,000, canola 260,000-350,000, sunflower 17,000-23,000 and cotton 28,000-55,000 plants per acre (Cheikh et al. (2003) U.S. patent application No. 20030101479). A desirable higher population density for each of these examples, as well as other valuable species of plants, would be at least 10% higher than the average prevailing density or yield. In particular, the creation of dwarf varieties can enable planting at high densities.

“Rootstock” refers to a lower portion of a plant comprising the roots and a portion of the stem which is fused through grafting to an aerial portion of the plant (the “Scion”), the latter generally having a different genotype. The grafting method allows for root beneficial and shoot beneficial phenotypes to be combined in the same plant by bringing together plant parts with different genetic compositions.

### Desirability of Dwarf Crop Plants

In many cases it is advantageous to grow smaller plants. For example, plants with smaller stature may be more resistant to damage from wind and rain, have improved lodging resistance, or may be more resistant to heat, low humidity, or water deficit. Dwarf plants are also of significant interest to the ornamental horticulture industry, and particularly for home garden applications for which space availability may be limited.

Growers of grain crops, such as wheat, rice, corn, and sorghum, often seek dwarf varieties in order to improve Harvest Index. Harvest index is the ratio of the total grain production of a crop to its biomass and high values are sought so as to capture as much grain yield as possible per area of land. In particular, trait developers are seeking “short corn” varieties with increased harvest index. Application of the inventions detailed herein can address this need.

Growers of fruit trees routinely prune and trim their trees; more compact dwarfed fruit tree varieties plants require less care in this regard. A large vegetative mass and excessive height is typically not desired in fruit trees since the plant expends greater energy on vegetative growth at the expense of fruit yield. It is also more challenging to harvest fruit from bulky, tall trees than those with a short stature. Thus, shorter dwarf varieties are typically desired.

Dwarf plants may exhibit an altered phenotype, as compared to a wild-type plant or a plant of typical stature, characterized by at least one of the following attributes: a) altered auxin transport, b) slower auxin transport, c) reduced apical dominance, d) an altered xylem/phloem ratio, e) an increased number of phloem elements, f) smaller phloem elements, g) thicker bark, h) a bushier habit, i) reduced root mass, j) reduced vigor, k) less vegetative growth, 1) earlier termination of shoot growth, m) earlier competence to flower, n) precocity, o) earlier phase change, p) smaller canopy, q) reduced stem circumference, r) reduced branch diameter, s) fewer sylleptic branches, t) shorter sylleptic branches, u) more axillary flowers, v) an earlier terminating primary axis, w) earlier terminating secondary axes, and x) shorter internode length y) reduced scion mass, or z) smaller size (see patent publication US20180371481, 2018, Foster).

The plant may be a root stock that is grafted to a scion to produce a plant that exhibits a dwarf phenotype when grown to maturity.

In contrast to the above examples, growers of commercial forestry plantations often desire trees that have long straight main stems with minimal branching, which yield higher quality timber. In these instance, the opposite of a dwarf phenotype is desired.

### ETTIN/ARF3

ETTIN, or ARF3, is of particular interest as a modulator of plant stature because it is a well characterized transcriptional regulator or auxin responses. Furthermore, an ortholog of ETTIN has been reported to be up-regulated in apple varieties ‘M9’ and ‘M27’ relative to vigorous rootstocks. (US20180371481, 2018, Foster). However, it has not previously been recognized ETTIN can be upregulated in apple and other fruit trees by mutating a uORF in order to produce a dwarf trait.

ARF3 is a member of a large family of Auxin Response Factors, transcription factors that activate or repress downstream genes in response to auxin. ARF3/ETTIN was first discovered as a gene required for normal patterning of floral organs in Arabidopsis (Sessions and Zambryski 1995. Development 121 (5): 1519-1532; Sessions, Nemhauser et al. 1997. Development 124 (22): 4481-4491. It was later discovered that ARF3 and the transcription factor KANADI mediate both auxin flow and organ polarity, which includes vascular patterning (Pekker, Alvarez et al. 2005. Plant Cell Online 17 (11): 2899-2910; Izhakia and Bowman 2007. Plant Cell 19:495-508; Kelley, Arreola et al. 2012 . . . . Development 139 (6): 1105-1109. ARF3 also has a key role in promoting phase change (transition to flowering), increased ARF3 expression leads to earlier flowering, loss of ARF3 function delays flowering. (Fahlgren, Montgomery et al. 2006. Current Biol. 16 (9): 939-944; Hunter, Willmann et al. 2006. Development 133 (15): 2973-2981). (US20180371481, 2018, Foster).

Auxin response factors bind specifically to the DNA sequence 5′-TGTCTC-3′ found in the auxin-responsive promoter elements (AuxREs and could potentially act as transcriptional activator or repressors. Formation of heterodimers with Aux/IAA proteins may also alter their ability to modulate early auxin response genes expression. Involved in the establishment or elaboration of tissue patterning during gynoecial development (www.ncbi.nlm.nih.gov/protein/023661.2). Thus, a further means to produce a dwarf phenotype is through mutation of residues in the domain of the ARF3 protein that interacts with Aux/IAA proteins.

uORFs

An upstream open reading frame (uORF) is a member of a class of small, conserved ORFs located upstream of protein-coding major ORFs (mORFs) in the 5′-untranslated regions (5′UTR) of mRNAs. uORFs act as cis acting elements that modify the activity of a downstream sequence that encodes a polypeptide. As such, they offer a novel opportunity to activate the expression of the longer downstream open reading frames encoding polypeptides of interest, through gene editing approaches that introduce mutations into the uORF sequences. Upregulation of the level of a target polypeptide can thereby be achieved by modulating the activity, or expression, for example, by knocking-out, of a negatively acting uORF upstream of the sequence encoding the polypeptide. Not all eukaryotic genes contain uORFs but hitherto the barrier to the aforementioned approach has been in identifying the uORF sequences; existing algorithms often fail to accurately identify these elements due to their short length and also given that they are often initiated via non-AUG start codons (Hellens et al., 2016. Trend Plant Sci. 21:317-328). uORFs are regulatory elements that are prevalent in eukaryotic mRNAs. uORFs are located upstream of protein-coding major ORFs (also known as main ORFs or mORFs) in the 5′-untranslated regions (5′UTR) of mRNAs. In some instances, uORFs are believed to modulate the translation initiation rate of downstream coding sequences (CDSs) by sequestering ribosomes. In other cases, uORFs encode evolutionarily conserved short peptides that function as cis-acting repressor peptides of the downstream mORF. In many cases the actual presence of a uORF is strongly conserved across species. Thus, once a uORF has been identified in a target locus from a given species, the homologous locus from another species will typically also contain a uORF and be subject to uORF repression. Identification of the uORFs from the species described herein (SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240) therefore provides a roadmap for identifying the uORF containing ETTIN homologs from other target crops based on homology searches for the polypeptides encoded by the mORFs at those loci.

A small number of examples have been described in plants whereby mutation of a uORF derepresses a downstream mORF encoding a polypeptide of interest, following the identification of the uORF in a first species. An example concerns the GGP clade of proteins which regulate ascorbate levels. A desirable trait comprising increased ascorbate levels was identified through overexpression of the gene in Arabidopsis; that locus contains a uORF, and the equivalent homologous gene in a target crop can be activated to obtain the same desired trait by mutation of the uORF in the crop gene, which will typically reside at a similar position upstream of the mORF in the homologous locus of the target crop. A specific instance concerns editing the uORF of LsGGP2, which encodes a key enzyme in vitamin C biosynthesis in lettuce, which was targeted based on the homologous gene having been demonstrated as being subject to uORF control in Arabidopsis by Laing et al (Liang et al., Plant Cell. 2015 March; 27 (3): 772-786). Editing the uORF of the lettuce homolog not only increased oxidation stress tolerance, but also increased ascorbate content by about 150% (Zhang et al. Nature Biotechnol. volume 36, pages 894-898 (2018).

Genome-wide studies have revealed the widespread regulatory functions of uORFs in different species in different biological contexts (Zhang et al. 2019. Trends Biochem. Sci. 44:782-794. doi: 10.1016/j.tibs.2019.03.002). A given uORF may act as a translational control element for regulating expression of its associated downstream major open reading frame (mORF). The translational regulation of mORFs by highly conserved uORFs in response to cellular metabolite levels has been documented in plant studies (Hayden C. A. and Jorgensen R. A. 2007. BMC Biol. 5:32; Tran M. K., et al. 2008. BMC Genomics 9:361).

Various methods to identify uORFs in eukaryotes have been described. For example, to identify conserved peptide uORFs, Hayden and Jorgensen created “uORF-Finder”, a Perl program that compares the mORF amino acid sequence of cDNAs from one collection with the mORF sequences of another species' collection to identify putative mORF homologs, and then compares uORFs in the 5′ UTRs of the two paired sequences to identify uORFs with conserved amino acid sequences (Hayden and Jorgensen, 2007. BMC Biology 5:32). By comparing full-length cDNA sequences from Arabidopsis and rice, distinct homology groups of conserved peptide uORFs are so identified. Skarshewski et al. describe the use of “uPEPperoni”, an online tool for upstream open reading frame location and analysis of transcript conservation (Skarshewski, A., et al. 2014. BMC Bioinform. 15:36. doi: 10.1186/1471-2105-15-36).

Rather than making use of bioinformatics-based analysis, Ingolia et al. describe methods for ribosome profiling: identifying uORFs by evaluating ribosome occupancy of upstream open reading frames and other sequences. See, for example, U.S. Pat. No. 9,677,068; Ingolia N. T., 2014. Cell Reports 8:5, 1365-1379. See also Ingolia N. T. 2011. Cell 11; 147:789-802 in which the authors describe how the majority of putative lincRNAs contain regions of high translation comparable to protein-coding genes. Specific start sites marked by harringtonine followed by ribosome footprints extended to the first in-frame stop codon. The majority of novel near-cognate initiation sites detected drive the translation of uORFs. This is consistent with the high level of translation that is observed on many 5′ UTRs as opposed to 3′ UTRs, which are almost devoid of ribosomes.

### Introduction of Targeted Genetic Modifications Through Gene Editing

A preferred method of practicing the invention is to use genome editing to produce a “targeted genetic modification” to produce a “non-naturally occurring allele” as referenced herein. The terms “genome editing”, “genome edited”, “genome modified”, “genetically modified” are used interchangeably to describe plants with specific DNA sequence changes in their genomes wherein those DNA sequence changes include changes of specific nucleotides, the deletion of specific nucleotide sequences or the insertion of specific nucleotide sequences.

As used herein, a technique for introducing a “targeted genetic modification” refers to any method, protocol, or technique that allows the precise and/or targeted editing at a specific location (also referred to a “locus” or “native locus” in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas system), a TALE-endonuclease (TALEN), a recombinase, or a transposase. CRISPR is an acronym for clustered, regularly interspaced, short, palindromic repeats and Cas an abbreviation for CRISPR-associated protein; for a review, see Khandagal & Nadal, Plant Biotechnol. Rep., 2016, 10, 327. Engineered meganucleases, zinc finger nucleases (ZFN), transcription activator-like effector nucleases (TALENs) can also be used. US Patent Application 2016/0032297 provides detailed methodology for these methods.

Genome editing tools can accurately change the architecture of a genome at specific target locations. These tools can be efficiently used for the generation of plants with high crop yields, desired alterations in composition, and resistance to biotic and abiotic stresses. It may be challenging to achieve all desired modifications using a particular genome editing tool. Thus, multiple genome editing tools have been developed to facilitate efficient genome editing. Some of the major genome editing tools used to edit plant genomes are: homologous recombination (HR), zinc finger nucleases (ZFNs), transcription activator-like effector nucleases (TALENs), pentatricopeptide repeat proteins (PPRs), the CRISPR/Cas9 system, RNA interference (RNAi), cisgenesis, and intragenesis. In addition, site-directed sequence editing and oligonucleotide-directed mutagenesis have the potential to edit the genome at the single-nucleotide level. Recently, adenine base editors (ABEs) have been developed to mutate A-T base pairs to G-C base pairs. ABEs use deoxyadeninedeaminase (TadA) with catalytically impaired Cas9 nickase to mutate A-T base pairs to G-C base pairs. A summary of these methods an applicability is provided by Mohanta et al., Genes (Basel). 2017 December; 8 (12): 399. Gene editing techniques are now available that use a variety of alternative CAS enzymes to CAS9

Such genome editing methods encompass a wide range of approaches to precisely remove genes, gene fragments, to alter the DNA sequence of coding sequences or control sequences, or to insert new DNA sequences into genes or protein coding regions to reduce or increase the expression of target genes in plant genomes (Belhaj, K. 2013, Plant Methods, 9, 39; Khandagale & Nadal, 2016, Plant Biotechnol Rep, 10, 327). Preferred methods involve the in vivo site-specific cleavage to achieve double stranded breaks in the genomic DNA of the plant genome at a specific DNA sequence using nuclease enzymes and the host plant DNA repair system. Multiple approaches are available for producing double stranded breaks in genomic DNA, and thus achieve genome editing, including the use of the CRISPR/Cas system.

An extensive overview of the CRISPR/Cas system and useful applications thereof can be found at www.addgene.org/guides/crispr/

The CRISPR/Cas genome editing system provides flexibility in targeting specific sequences for modification within the genome and enables the execution of a range of different edits including the activation or upregulation of target loci or the knock-out of target loci. The method relies on providing the Cas enzyme and a short guide RNA “gRNA” containing a short guide sequence (~20 bp), with sequence complementarity to the target DNA sequence in the plant genome, for example, a guide RNA with complementarity to a uORF. Depending on the type of Cas enzyme, alternatively a DNA, an RNA/DNA hybrid, or a double stranded DNA guide polynucleotide can be used. The guide portion of this guide polynucleotide directs the Cas enzyme to the desired cut site for cleavage with a recognition sequence for binding the Cas enzyme.

The target in the plant genome can be any ~20-mer nucleotide DNA sequence, provided that the sequence is unique compared to the rest of the genome and also that the target is present immediately adjacent to a Protospacer Adjacent Motif (PAM). The PAM sequence serves as a binding signal for the editing enzyme, but the exact sequence depends on which Cas protein is being used. A list of Cas proteins and PAM sequences can be found at www.addgene.org/guides/crispr/#pam-table

The simplest application of CRISPR/Cas is to produce knockout or loss of function alleles in a target locus. The gRNA targets the Cas enzyme to a specific locus in the genome, which then produces a double stranded break. The resulting DSB is then repaired by one of the general repair pathways present in the cell. This typically causes small nucleotide insertions or deletions (indels) at the DSB site. In most cases, small indels in the target DNA result in amino acid deletions, insertions, or frameshift mutations leading to premature stop codons within the open reading frame (ORF) of the targeted gene. The ideal result is a loss-of-function mutation within the targeted gene. However, the strength of the knockout phenotype for a given mutant cell must be validated experimentally, for example for testing for the presence of transcript from the target ORF by RT-PCR or hybridization-based approaches. These features make the CRISPR/Cas system a suitable tool for knockout of uORFs.

CRISPR/Cas can also be used to produce more sophisticated changes to the native sequence at targeted loci in the genome. This can involve inserting sequences, replacing sequences, or editing specific bases so as to insert or create new domains within a polypeptide encoded at a desired locus. One way to introduce such changes is to make use of the high fidelity but low efficiency high fidelity homology directed (HDR) repair pathway within the cell. In order to make such precise modifications using HDR, a DNA repair template incorporating the desired genome modification that the practitioner desires to create at the target locus must be delivered into the cell type of interest with the gRNA(s) and Cas9 or Cas9 nickase. The repair template must contain the desired edit as well as additional homologous sequence immediately upstream and downstream of the target (termed left & right homology arms). The length of each homology arm is dependent on the size of the change being introduced, with larger insertions requiring longer homology arms. Since the efficiency of Cas9 cleavage is relatively high and the efficiency of HDR is relatively low, a large portion of the Cas9-induced DSBs will be repaired to produce edits not comprising the specific desired change. Thus, an additional confirmation/screening step is required to select one of more cells from the edited population that contain the desired change. These cells then can be regenerated into a population of cells, a tissue, organ or whole plant or plant population. Such selection can be achieved by incorporating a marker sequence into the edit, which is readily screened or by PCR or hybridization-based methods.

CRISPR-related gene editing systems can also be deployed to change specific bases without the need for double stranded breaks. Such approaches are referred to in the art as “base editing” systems. Using these systems, the skilled practitioner can create a targeted genetic modification comprising an amino acid substitution or the creation of start or stop codon.

To avoid relying on HDR, which has low efficiency, researchers have developed two classes of base editors: cytosine base editors (CBEs) and adenine base editors (ABEs). Cytosine base editors are created by fusing Cas9 nickase or catalytically inactive “dead” Cas9 (dCas9) to a cytidine deaminase like APOBEC. As with traditional CRISPR techniques, base editors are targeted to a specific locus by a gRNA, and they can convert cytidine to uridine within a small editing window near the PAM site. Uridine is subsequently converted to thymidine through base excision repair, creating a C to T change. Likewise, adenosine base editors have been engineered to convert adenosine to inosine, which is treated like guanosine by the cell, creating an A to G change.

Adenine DNA deaminases do not exist in nature, but these enzymes have been created by directed evolution of the Escherichia coli TadA, a tRNA adenine deaminase. Like cytosine base editors, the evolved TadA domain is fused to a Cas9 protein to create the adenine base editor. Both types of base editors are available with multiple Cas9 variants including high fidelity Cas9's. Further advancements have been made by optimizing expression of the fusions, modifying the linker region between Cas variant and deaminase to adjust the editing window, or adding fusions that increase product purity such as the DNA glycosylase inhibitor (UGI) or the bacteriophage Mu-derived Gam protein (Mu-GAM).

While many base editors are designed to work in a very narrow window proximal to the PAM sequence, some base editing systems create a wide spectrum of single-nucleotide variants (somatic hypermutation) in a wider editing window, and are thus well suited to directed evolution applications. Examples of these base editing systems include targeted AID-mediated mutagenesis (TAM) and CRISPR-X, in which Cas9 is fused to activation-induced cytidine deaminase (AID).

Other CRISPR systems, specifically the Type VI CRISPR enzymes Cas13a/C2c2 and Cas13b, target RNA rather than DNA. Fusing a hyperactive adenosine deaminase that acts on RNA, ADAR2 (E488Q), to catalytically dead Cas13b creates a programmable RNA base editor that converts adenosine to inosine in RNA (termed REPAIR). Since inosine is functionally equivalent to guanosine, the result is an A->G change in RNA. The catalytically inactive Cas13b ortholog from Prevotella sp., dPspCas13b, does not appear to require a specific sequence adjacent to the RNA target, making this a very flexible editing system. Editors based on a second ADAR variant, ADAR2 (E488Q/T375G), display improved specificity, and editors carrying the delta-984-1090 ADAR truncation retain RNA editing capabilities and are small enough to be packaged in AAV particles.

In the context of this disclosure, it is recognized that the term Cas nuclease includes any nuclease which site-specifically recognizes CRISPR sequences based on gRNA or DNA sequences and includes Cas9, Cpf1 and others described below. Many authors have identified that CRISPR/Cas genome editing, is a preferred way to edit the genomes of complex organisms (Sander & Joung, 2013, Nat Biotech, 2014, 32, 347; Wright et al., 2016, Cell, 164, 29) including plants (Zhang et al., 2016, Journal of Genetics and Genomics, 43, 151; Puchta, E L, 2016, Plant J., 87, 5; Khandagale & Nadaf, 2016, PLANT BIOTECHNOL REP, 10, 327). US Patent Application 2016/020822 provides extensive description of the materials and methods useful for genome editing in plants using the CRISPR/Cas9 system and describes many of the uses of the CRISPR/Cas9 system for genome editing of a range of gene targets in crops.

It is further recognized that many variations of the CRISPR/Cas system can be used for applying the invention herein, including the use of wild-type Cas9 from Streptococcus pyogenes (Type II Cas) (Barakate & Stephens, 2016, Frontiers in Plant Science, 7, 765; Bortesi & Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187). Other examples include Tru-gRNA/Cas9 in which off-target mutations are significantly decreased (Fu et al., 2014, Nature biotechnology, 32, 279; Osakabe et al., 2016, Scientific Reports, 6, 26685; Smith et al., 2016, Genome biology, 17, 1; Zhang et al., 2016, Scientific Reports, 6, 28566), a high specificity Cas9 (mutated S. pyogenes Cas9) with little to no off-target activity (Kleinstiver et al., 2016, Nature 529, 490; Slaymaker et al., 2016, Science, 351, 84). Further variations comprise the Type I and Type III systems in which multiple Cas proteins are expressed to achieve editing (Li et al., 2016, Nucleic acids research, 44:e34; Luo et al., 2015, Nucleic acids research, 43, 674), the Type V Cas system using the Cpf1 enzyme (Kim et al., 2016, Nature biotechnology, 34, 863; Toth et al., 2016, Biology Direct, 11, 46; Zetsche et al., 2015, Cell, 163, 759), DNA-guided editing using the NgAgo Argonaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819). A unique nuclease Cpf1, an alternative to Cas9 has advantages over the Cas9 system in reducing off-target edits which creates unwanted mutations in the host genome. Examples of crop genome editing using the CRISPR/Cpf1 system include rice (Tang et. al., 2017, Nature Plants 3, 1-5; Wu et. al., 2017, Molecular Plant, Mar. 16, 2017) and soy bean (Kim et., al., 2017, Nat Commun. 8, 14406). Other authors have described the use of Argonaute related proteins as an alternative to CRISPR systems for gene editing (Hegge et al. Nature reviews Microbiology. 2017. Epub 2017 Jul. 25. pmid: 28736447; Swarts et al. Nucleic acids research. 2015; 43 (10): 5120-9. Epub 2015 May 1. pmid: 25925567; Swarts et al. Nature. 2014; 507 (7491): 258-61. Epub 2014 Feb. 18. pmid: 24531762. See also PCT Application Number PCT/US2019/025163 and/or Publication Number WO2019204266A1.

Detailed methodologies for gene editing in plants to create new crop traits, including the selection of cells containing the desired edits, and methods for introducing the CRISPR system components into an initial target plant cell are set forth in published patent application WO2019195157. As specified therein, the “guide polynucleotide” in a CRISPR system also relates to a polynucleotide sequence that can form a complex with a Cas endonuclease and enables the Cas endonuclease to recognize and optionally cleave a DNA target site. The guide polynucleotide can be a single molecule (i.e., a single guide RNA (gRNA) that is a synthetic fusion between a crRNA and part of the tracrRNA sequence) or two molecules (i.e. the crRNA and tracrRNA as found in natural Cas9 systems in bacteria). The guide polynucleotide sequence can be provided as an RNA sequence or can be transcribed from a DNA sequence to produce an RNA sequence. The guide polynucleotide sequence can also be provided as a combination RNA-DNA sequence (see for example, Yin, H. et al., 2018, Nature Chemical Biology, 14, 311). As used herein “guide RNA” sequences comprise a variable targeting domain, called the “guide”, complementary to the target site in the genome (for example, a guide RNA with complementarity to a uORF), and an RNA sequence that interacts with the Cas9 or Cpf1 endonuclease, called the “guide RNA scaffold”. A guide polynucleotide that solely comprises ribonucleic acids is also referred to as a “guide RNA”. As used herein the “guide target sequence” refers to the sequence of the genomic DNA adjacent to a PAM site, where the gRNA will bind to cleave the DNA. The “guide target sequence” is often complementary to the “guide” portion of the gRNA, however several mismatches, depending on their position, can be tolerated and still allow Cas mediated cleavage of the DNA. The method also provides introducing single guide RNAs (gRNAs) into plants. The single guide RNAs (gRNAs) include nucleotide sequences that are complementary to the target chromosomal DNA. The gRNAs can be, for example, engineered single chain guide RNAs that comprise a crRNA sequence (complementary to the target DNA sequence) and a common tracrRNA sequence, or as crRNA-tracrRNA hybrids. The gRNAs can be introduced into the cell or the organism as a DNA with an appropriate promoter, as an in vitro transcribed RNA, or as a synthesized RNA. Basic guidelines for designing the guide RNAs for any target gene of interest are well known in the art as described for example by Brazelton et al. (Brazelton, V. A. et al., 2015, GM Crops & Food, 6, 266-276) and Zhu (Zhu, L. J. 2015, Frontiers in Biology, 10, 289-296).

Published patent applications WO2019195157 and WO2019204266A1 also provides example of the types of mutation that can lead to increased activity of transcription factor polypeptides. These include mutations to the coding sequence that give rise to amino acid changes in the encoded protein.

In certain preferred embodiments of the present invention, the guide polynucleotide/Cas endonuclease system can be used to allow for the insertion or deletion of a promoter or promoter element, such as an enhancer element, upstream of the coding sequence of any one the polypeptide sequences of the invention, wherein the promoter insertion (or promoter element deletion) results in any one of the following or any one combination of the following: an elevated level of the polypeptide, a permanently activated gene locus, an increased promoter activity (increased promoter strength), an increased promoter tissue specificity, a decreased promoter tissue specificity, a new promoter activity, an extended window of gene expression, a modification of the timing or developmental progress of gene expression, a mutation of DNA binding elements and/or an addition of DNA binding elements.

The guide RNA/Cas endonuclease system can be used to allow for the insertion of a promoter element to increase the expression of the polypeptide sequences of this disclosure. Promoter elements, such as enhancer elements, are often introduced in promoters driving gene expression cassettes in multiple copies for trait gene testing or to produce transgenic plants expressing specific traits. Enhancer elements can be, but are not limited to, a 35S enhancer element (Benfey et al, EMBO J, August 1989; 8 (8): 2195-2202). In some plants (events), the enhancer elements can cause a desirable phenotype, a yield increase, or a change in expression pattern of the trait of interest that is desired. It may be desired to remove the extra copies of the enhancer element while keeping the trait gene cassettes intact at their integrated genomic location. The guide RNA/Cas endonuclease can be used to remove the unwanted enhancing element from the plant genome. A guide RNA can be designed to contain a variable targeting region targeting a target site sequence of 12-30 bps adjacent to a NGG (PAM) in the enhancer. The Cas endonuclease can make cleavage to insert one or multiple enhancers.

To repress the function of a target polypeptide encoding locus, the promoter elements to be deleted can be, but are not limited to, promoter core elements, promoter enhancer elements or 35 S enhancer elements (CaMV35S enhancers (Benfey et al, EMBO J, August 1989; 8 (8): 2195-2202)). The promoter or promoter fragment to be deleted can be endogenous, artificial, pre-existing, or transgenic to the cell that is being edited. Preferably the promoter element is endogenous to the cell that is being edited. These approaches, removing promoters or promoter elements, can be applied to knock-out polypeptides of interest.

In a further embodiment, the targeted genetic modification of interest is an intron site of any one of the polypeptide sequences of the disclosure, wherein the modification consists of inserting an intron enhancing motif into the intron which results in modulation of the transcriptional activity of the gene containing said intron. In yet another embodiment, methods provide for modifying alternative splicing sites of any one of the polypeptide sequences of the disclosure resulting in enhanced production of the functional gene transcripts and polypeptides (proteins).

In additional embodiments, the modification of the polypeptide sequences of the disclosure includes editing the intron borders of alternatively spliced genes to alter the accumulation of splice variants. In other embodiments, the guide polynucleotide/Cas endonuclease system can be used to modify or replace a coding sequence of the polypeptide in the genome of a plant cell, wherein the modification or replacement results in any one of the following, or any one combination of the following: an increased protein activity, an increased protein functionality, a site specific mutation, a protein domain swap, a protein knock-out, a new protein functionality, insertion or creation of a transcriptional activation domain, insertion or creation of a transcriptional repression domain, or a modified protein functionality.

Delivery of Gene Editing Components into Plant Cells and Plants

Sandhya et al., J Genet Eng Biotechnol. 2020 December; 18:25. Published online 2020 Jul. 7. doi: 10.1186/s43141-020-00036-8, present methods for delivering gene editing tools such CRISPR/Cas9 components into plants to execute the gene editing process. The effective delivery of CRISPR/Cas9 components, including the guide sequence, the Cas9, and where applicable a DNA-repair template containing the desired sequence edit, into plant cells is critical for editing to be efficient. The practitioner can select from a variety of delivery methods to introduce the gene editing components into plant cells. These include Agrobacterium-mediated, bombardment or biolistic method, floral-dip, and PEG-mediated protoplast transformation. Additional methods include nanoparticle and pollen magnetofection-mediated delivery systems (Kwak et ai., 2019, Nature Nanotechnology, DOI 10.1038/S41565-019-0375-4) (Demirer et al, 2019, Nature Nanotechnology, DOI 10.1038/S41565-019-0382-5) can be used. CRISPR constructs can be coated onto gold particles for gene gun mediated introduction into plant cells, CRISPR constructs can be transfected into protoplasts using PEG, or introduced via an Agrobacterium strain harboring a CRISPR vector. Components may also be introduced via floral dip (Castel et al., PLOS One. 2019; 14 (1):e0204778) or a pollen-tube tube pathway-based method. In the next step, a plant cell containing targeted genetic modification produced by the introduced CRISPR system is selected and regenerated into an explant and then a plant tissue or whole plant. In some instances, this procedure involves selecting explants harboring the genome edit on selection plates and regenerating a whole plant. Finally, PCR and Sanger sequencing are generally used for confirmation that the desired sequence edit has been successfully introduced into the selected plant. The selected plant is then examined to confirm that it exhibits the target trait of interest that was initially sought by introducing the genome modification.

Sandhya et al., J Genet Eng Biotechnol. 2020 December; 18:25. Published online 2020 Jul. 7. doi: 10.1186/s43141-020-00036-8, provide tables showing which methods can be successfully applied to particular crops. For example, the following plants can all be successfully gene edited using PEG mediated delivery of CRISPR system components: Apple, Brassica oleracea, Brassica rapa, Citrullus lanatus, Glycine max, Grapevine, Oryza sativa, Petunia, Physcomitrella patens, Solanum lycopersicum, Triticum aestivum, and Zea mays. By way of further example, the following plants can all be successfully gene edited using particle bombardment mediated delivery of CRISPR system components: Glycine max, Hordeum vulgare, Oryza sativa, Triticum aestivum and Zea mays. By way of a further example, the following plants can all be successfully gene edited using particle bombardment mediated delivery of CRISPR system components: Arabidopsis thaliana, Banana, Citrus sinensis, Cucumis sativum, Glycine max, Kiwi fruit, Lotus japonicus, Marchantia polymorpha, Medicago truncatula, Nicotiana benthamaina, Nicotiana tabacum, Oryza sativa, Populus, Salvia miltiorrhiza, Solanum lycopersicum, Sorghum bicolor, Triticum aestivum and Zea mays.

Activation of ETTIN and its Homologs Through Targeted Genetic Modifications that Comprise the Mutation of uORFs

Upstream ORFs (uORFs) encode short mRNAs that are encoded within the 5′ UTR of a gene encoding a regulatory protein of interest (the coding sequence of which is sometimes referred to as the main ORF or mORF). The uORF is typically out-of-frame with the main coding sequence of the gene of interest, which in an embodiment of the invention herein is a transcription factor gene or ETTIN protein-encoding gene. A substantial proportion of eukaryotic mRNAs contain uORFs in the 5′ leader sequence preceding the main functional protein-encoding ORF (Kochetov, 2008, BioEssays 30:683-691). uORFs often encode short peptides which negatively regulate the activity of the regulatory proteins encoded by the genes of which they are upstream. Furthermore, uORFs sometimes initiate at a non-canonical codon (e.g., ACG rather than AUG) and the encoded peptide is often much less than 100 residues in length. These features make uORFs challenging to identify via automated bioinformatic searches, and experimentation is typically needed to confirm that a putative uORF functions as a negative regulator of a downstream coding sequence. For example, Laing et al., 2015, Plant Cell 27 (3) 772-786; DOI: 10.1105/tpc.114.133777, removed a uORF that encodes a 60- to 65-residue peptide in the upstream region of GGP in lettuce, and showed this was sufficient to deliver a trait comprising increase levels of ascorbate. In fact, the peptides encoded by uORFs are very short indeed in some instances; for example, in humans, a functional peptide of only 6 amino acids was identified as being encoded by a uORF. Several plant uORFs have been shown to modulate mORF translation in response to the levels of various key metabolites within the cell (e.g., polyamines, sucrose, phosphocholine and ascorbate. A proposed function of several of the peptides encoded by these uORFs is to slow or stall the ribosomes and as a consequence limit translation of the downstream main ORF which encodes to regulatory protein (see Hellens et al., 2016, Trends in Plant Science, Vol. 21, No. 4 pp 317. dx.doi.org/10.1016/j.tplants.2015.11.005, and references therein).

Recently, it has been proposed that gene editing of uORFs may offer a general approach to activate crop genes to produce traits of interest in a highly targeted manner (Zhang and Voytas, May 2019, National Science Review, Volume 6, Issue 3, Page 391, doi.org/10.1093/nsr/nwy123). In particular, this can avoid many of the drawbacks associated with traditional approaches, which often involve large insertions of foreign DNA fragments, such as sequences of strong promoters, enhancers or engineered artificial transcription activators, in the genome. Indeed, many of the problems associated with genetic modifications that comprise transgene integrations, including lack of consumer acceptance of GM products, may be eliminated in a next generation of crop traits produced through knock-out of uORFs in regulator genes by targeted gene editing.

An increasing number of uORFs are being identified in the upstream regions of genes that encode transcriptional regulators and in many cases the uORF and/or its encoded short peptide appear to be controlled by a metabolic signal (van der Horst 2020, Plant Physiol. 2020 January; 182 (1): 110-122, Published online 2019 Aug. 26. doi: 10.1104/pp. 19.00940 and references therein). These include these the S1-group bZIPs, including the (HG1) bZIP transcription factor, which controls amino acid and sugar metabolism and which in turn has its activity regulated by sucrose. SAC51 (HG15) is bHLH transcription factor which is involved in xylem differentiation and regulated in response to thermospermine. Another example is the HsfB1/TBF1 (HG18) HSF transcription factor which is involved in heat tolerance and growth-to-defense transition which is regulated by galactinol.

A further example of transcription factor regulation by uORFs concerns a group of uORF-containing genes identified in the AUXIN RESPONSE FACTOR transcription factor family, of which ETTIN, the subject of this application, is a member (Hellens et al., 2016, Trends in Plant Science, Vol. 21, No. 4 pp 317; Schepetilnikov, M. et al. 2013, EMBO J. 32, 1087-1102; Nishimura, T. et al., 2005, Plant Cell 17, 2940-2953; Zhou, F. et al., 2010, BMC Plant Biol. 10, 193).

### Identifying Homologs (Including Orthologs and Paralogs) for Targeted Genetic Modification

A homolog of a polypeptide is a related polypeptide that possesses an equivalent or similar function to a given polypeptide and which can be subjected to a targeted genetic modification to deliver an equivalent or similar trait to a given polypeptide.

Homologous sequences as described herein can comprise orthologous or paralogous sequences. As used herein, a paralog is a homolog from the same species and an ortholog is a homolog from a different species. Several different methods are known by those of skill in the art for identifying and defining these functionally homologous sequences. General methods for identifying orthologs and paralogs, including phylogenetic methods, sequence similarity and hybridization methods, are described herein; an ortholog or paralog, including equivalogs, may be identified by one or more of the methods described below.

As described by Eisen, (1998) 10.1101/gr.8.3.163 Genome Res. 8:163-167), evolutionary information may be used to predict gene function. It is common for groups of genes that are homologous in sequence to have diverse, although usually related, functions. However, in many cases, the identification of homologs is not sufficient to make specific predictions because not all homologs have the same function. Thus, an initial analysis of functional relatedness based on sequence similarity alone may not provide one with a means to determine where similarity ends and functional relatedness begins. Fortunately, it is well known in the art that protein function can be classified using phylogenetic analysis of gene trees combined with the corresponding species. Functional predictions can be greatly improved by focusing on how the genes became similar in sequence (i.e., by evolutionary processes) rather than on the sequence similarity itself (Eisen, 1998) supra. In fact, many specific examples exist in which gene function has been shown to correlate well with gene phylogeny (Eisen, 1998) supra. Thus, “[t]he first step in making functional predictions is the generation of a phylogenetic tree representing the evolutionary history of the gene of interest and its homologs. Such trees are distinct from clusters and other means of characterizing sequence similarity because they are inferred by techniques that help convert patterns of similarity into evolutionary relationships After the gene tree is inferred, biologically determined functions of the various homologs are overlaid onto the tree. Finally, the structure of the tree and the relative phylogenetic positions of genes of different functions are used to trace the history of functional changes, which is then used to predict functions of [as yet] uncharacterized genes” (Eisen, 1998) supra.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22:4673-4680; Higgins et al. (1996) Methods Enzymol. 266:383-402). Groups of similar genes can also be identified with pair-wise BLAST analysis (Feng and Doolittle (1987) J. Mol. Evol. 25:351-360). For example, a clade of very similar MADS domain transcription factors from Arabidopsis all share a common function in flowering time (Ratcliffe et al. (2001) Plant Physiol. 126:122-132), and a group of very similar AP2 domain transcription factors from Arabidopsis are involved in tolerance of plants to freezing (Gilmour et al. (1998) Plant J. 16:433-442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001), in Bioinformatics: Sequence and Genome Analysis, Cold Spring Harbor Laboratory Press, Cold Spring Harbor, New York, page 543).

Transcription factor gene sequences (ETTIN is an example of a transcription factor) are conserved across diverse eukaryotic species lines (Goodrich et al. (1993) Cell 75:519-530; Lin et al. (1991) Nature 353:569-571; Sadowski et al. (1988) Nature 335:563-564). Plants are no exception to this observation; diverse plant species possess transcription factors that have similar sequences and functions. Speciation, the production of new species from a parental species, gives rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22:4673-4680); Higgins et al. (1996) Methods Enzymol. 266:383-402) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

By using a phylogenetic analysis, one skilled in the art would recognize that the ability to predict similar functions conferred by closely-related polypeptides is predictable. This predictability has been confirmed by our own many studies in which we have found that a wide variety of polypeptides have orthologous or closely-related homologous sequences that function as does the first, closely-related reference sequence.

The polypeptides sequences belong to distinct clades of polypeptides that include members from diverse species. In each case, most or all of the clade member sequences derived from both dicots and monocots have been shown to confer increased tolerance to one or more abiotic stresses when the sequences were overexpressed, and hence will likely increase yield and or crop quality. These studies each demonstrate that evolutionarily conserved genes from diverse species are likely to function similarly (i.e., by regulating similar target sequences and controlling the same traits), and that polynucleotides from one species may be transformed into closely-related or distantly-related plant species to confer or improve traits.

At the nucleotide level, homologs of the sequences of the invention will typically share at least about 30% or 40%, 41%, 42%, 43%, 44%, 45%, 46%, 47%, 48%, 49%, 50%, 51%, 52%, 53%, 54%, 55%, 56%, 57%, 58, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% nucleotide sequence identity to one or more of the listed full-length sequences, or to a region of a listed sequence excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

At the polypeptide level, homologs of sequences of the invention will typically share preferably at least about 30%, or 35%, or 40%, 41%, 42%, 43%, 44%, 45%, 46%, 47%, 48%, 49%, 50%, 51%, 52%, 53%, 54%, 55%, 56%, 57%, 58, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% sequence identity to one or more of the listed full-length sequences. Within the conserved domain, which is often a DNA binding domain, homologs of sequences of the invention will typically share preferably at least about 60%, about 65%, about 70% or about 80% sequence identity, and more preferably about 85%, about 90%, about 95% or about 97% or more sequence identity to the conserved domains of the listed sequences.

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp (1988) Gene 73:237-244). The clustal algorithm groups sequences into clusters by examining the distances between all pairs. The clusters are aligned pairwise and then in groups. Other alignment algorithms or programs may be used, including ENTREZ, FASTA and BLAST, and which may be used to calculate percent similarity. These are available as a part of the GCG sequence analysis package (University of Wisconsin, Madison, WI), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Software for performing BLAST analyses is publicly available, e.g., through the National Center for Biotechnology Information (see www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410; Altschul (1993) J. Mol. Evol. 36:290-300. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, n=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). Unless otherwise indicated for comparisons of predicted polynucleotides, “sequence identity” refers to the % sequence identity generated from a protein sequence blast or a tblastx using the NCBI version of the algorithm at the default settings using gapped alignments with the filter “off” (see, for example, www.ncbi.nlm.nih.gov/).

Other techniques for alignment are described by Doolittle, R. F. (1996) Methods in Enzymology: Computer Methods for Macromolecular Sequence Analysis, vol. 266, Academic Press, Orlando, FL, USA. Preferably, an alignment program that permits gaps in the sequence is utilized to align the sequences. The Smith-Waterman is one type of algorithm that permits gaps in sequence alignments (see Shpaer (1997) Methods Mol. Biol. 70:173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (see, for example, Hein (1990) Methods Enzymol. 183:626-645) Identity between sequences can also be determined by other methods known in the art, e.g., by varying hybridization conditions (see Published U.S. patent application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al., 1997), PFAM, and other databases which contain previously identified and annotated motifs, sequences, and gene functions. Methods that search for primary sequence patterns with secondary structure gap penalties (Smith et al. (1992) Protein Engineering 5:35-51) as well as algorithms such as Basic Local Alignment Search Tool (BLAST; BLAST; Altschul (1993) J. Mol. Evol. 36:290-300; Altschul et al. (1990) J. Mol. Biol. 215:403-410), BLOCKS ((Henikoff and Henikoff (1991) Nucleic Acids Res. 19:6565-6572), Hidden Markov Models (HMM; Eddy (1996) Curr. Opin. Str. Biol. 6:361-365; Sonnhammer et al. (1997) Proteins 28:405-420), and the like, can be used to manipulate and analyze polynucleotide and polypeptide sequences encoded by polynucleotides. These databases, algorithms and other methods are well known in the art and are described in Ausubel et al. (1997; Short Protocols in Molecular Biology, John Wiley & Sons, New York, NY, unit 7.7) and in Meyers (1995; Molecular Biology and Biotechnology, Wiley VCH, New York, NY, p 856-853), and in Meyers (1995; Molecular Biology and Biotechnology, Wiley VCH, New York, NY, p 856-853).

Furthermore, methods using manual alignment of sequences similar or homologous to one or more polynucleotide sequences or one or more polypeptides encoded by the polynucleotide sequences may be used to identify regions of similarity and conserved domains characteristic of a particular transcription factor family, for example, sequences related to ETTIN. Such manual methods are well-known of those of skill in the art and can include, for example, comparisons of tertiary structure between a polypeptide sequence encoded by a polynucleotide that comprises a known function and a polypeptide sequence encoded by a polynucleotide sequence that has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed polypeptides may be directly synthesized or cloned using compositions provided by the present invention according to methods well known in the art. cDNAs can be cloned using mRNA from a plant cell or tissue that expresses one of the present sequences. Appropriate mRNA sources may be identified by interrogating Northern blots with probes designed from the present sequences, after which a library is prepared from the mRNA obtained from a positive cell or tissue. Polypeptide-encoding cDNA is then isolated using, for example, PCR, using primers designed from a presently disclosed gene sequence, or by probing with a partial or complete cDNA or with one or more sets of degenerate probes based on the disclosed sequences. The cDNA library may be used to transform plant cells. Expression of the cDNAs of interest is detected using, for example, microarrays, Northern blots, quantitative PCR, or any other technique for monitoring changes in expression. Genomic clones may be isolated using similar techniques to those.

This disclosure encompasses isolated nucleotide sequences that are phylogenetically and structurally similar to sequences listed in the Sequence Listing and can function in a plant by conferring a dwarf phenotype when gene edited or otherwise modified in a plant. One skilled in the art would predict that other similar, phylogenetically related sequences falling within the present clades of disclosed sequences would also perform similar functions when similarly upregulated or downregulated.

### Identifying Polynucleotides or Nucleic Acids by Hybridization

Polynucleotides homologous to those encoding the polypeptides sequences illustrated in the Sequence Listing and Table Ican be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking, and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (e.g., Sambrook et al. (1989) Molecular Cloning: A Laboratory Manual, 2nd Ed., Cold Spring Harbor Laboratory, Cold Spring Harbor, N.Y; Berger and Kimmel (1987) Guide to Molecular Cloning Techniques, Methods in Enzymology, vol. 152 Academic Press, Inc., San Diego, CA; and Anderson and Young (1985) “Quantitative Filter Hybridisation.” In: Hames and Higgins, ed., Nucleic Acid Hybridisation, A Practical Approach. Oxford, IRL Press, 73-111.

Encompassed by this disclosure are polynucleotide sequences that are capable of hybridizing to the polynucleotides that encode the claimed polypeptide sequences, including any of the polypeptides within the Sequence Listing, and fragments thereof under various conditions of stringency (see, for example, Wahl and Berger (1987) Methods Enzymol. 152:399-407; and Kimmel (1987) Methods Enzymol. 152:507-511). In addition to the nucleotide sequences that encode the polypeptides listed in the Sequence Listing, full length cDNA, orthologs, and paralogs of the polynucleotides encoding the present polypeptide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al., 1989 supra; Berger and Kimmel, 1987 supra, pages 467-469; and Anderson and Young, 1985 supra.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equations:

$$DNA - DNA : \qquad ( I )$$

$$Tm ( ° C . ) = 81.5 + 16.6 ( log ( Na + ] ) + 0.41 ( \% G + C ) - 0.62 ( \% formamide ) - 500 / L$$

$$DNA - RNA : \qquad ( II )$$

$$Tm ( ° C . ) = 79.8 + 18.5 ( log ( Na + ] ) + 0.58 ( \% G + C ) + 0.12 ( \% G + C ) 2 - 0.5 ( \% formamide ) - 820 / L$$

$$RNA - RNA : \qquad ( III )$$

$$Tm ( ° C . ) = 79.8 + 18.5 ( log ( Na + ] ) + 0.58 ( \% G + C ) + 0.12 ( \% G + C ) 2 - 0.35 ( \% formamide ) - 820 / L$$

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young, 1985, supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength, and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tm−5° C. to Tm−20° C., moderate stringency at Tm−20° C. to Tm−35° C. and low stringency at Tm−35° C. to Tm−50° C. for duplex>150 base pairs. Hybridization may be performed at low to moderate stringency (25-50° C. below Tm), followed by post-hybridization washes at increasing stringencies. Maximum rates of hybridization in solution are determined empirically to occur at Tm−25° C. for DNA-DNA duplex and Tm−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or Northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present polypeptides include, for example:
0.5×, 1.0×, 1.5×, or 2×SSC, 0.1% SDS at 50°, 55°, 60° or 65° C., or 6×SSC at 65° C.;
50% formamide, 4×SSC at 42° C.; or
0.5×SSC, 0.1% SDS at 65° C.;

with, for example, two wash steps of 10-30 minutes each. Useful variations on these conditions will be readily apparent to those skilled in the art. A formula for “SSC, 20×” may be found, for example, in Ausubel et al., Current Protocols in Molecular Biology, Ausubel et al. eds., Current Protocols, a joint venture between Greene Publishing Associates, Inc. and John Wiley & Sons, Inc., (supplemented through 2000).

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, U.S. patent application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a polypeptide known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences capable of hybridizing to the polynucleotide sequences that encode the claimed polypeptides, including any of the polypeptides within the Sequence Listing, and fragments thereof under various conditions of stringency (see, for example, Wahl and Berger, 1987, supra, pages 399-407; and Kimmel, 1987, supra). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

Identification of Homologs by the BLAST-Out and BLAST-Back Method, Followed by Generation or Selection of a uORF Mutation

One skilled in the art can apply the following method to identify a homolog in target (crop) species in which the practitioner wishes to generate a trait. The practitioner first selects a query polynucleotide from a query plant species that delivers a trait of interest, and BLASTs the protein sequence encoded by the query polynucleotide against the proteome, or translated DNA sequences, from the target (crop) species of interest. A particular crop protein sequence identified from the BLAST-out can be considered a homolog if (i) it matches the GID with a HSP of bit score 50 or better and (ii) when the protein is compared back to the set of protein sequences encoded by the genome of the plant from which the query sequence was obtained, and the protein is more similar to the protein encoded by the query polynucleotide, or any paralog of the query polynucleotide, than it is to any other protein sequence encoded by the genome of the query plant species. The practitioner may then target a genetic modification to upregulate or downregulate the identified (crop) protein to produce the desired trait, for example, through the modification of a uORF that may be found upstream of the polynucleotide encoding the identified protein. A particular embodiment of the inventions detailed herein involves selecting or generating so called “weak” non naturally occurring alleles, which result in only a moderate increase in the level of ETTIN homolog protein in the crop of interest. In particular, mutations which completely eliminate the activity of the uORF are less desirable, as these can produce very high levels of the ETTIN homolog protein (e.g., greater than 50% higher than the levels typically found in a wild-type plant) and often lead to developmental defects and/or a reduction in yield. Most favored are alleles which maintain the function of the uORF and result in an elevation in the level of the ETTIN homolog polypeptide to a level that is only between 5 and 50% higher than the level found in a wild-type control plant. Such desirable mutations are typically those which fall outside of the start codon as well as mutations which do not produce a frame shift in the uORF

### EXAMPLES

The invention, now being generally described, will be more readily understood by reference to the following examples, which are included merely for purposes of illustration of certain aspects and embodiments of the present invention and are not intended to limit the invention. It will be recognized by one of skill in the art that a genetic modification that is associated with a particular first trait may also be associated with at least one other, unrelated, and inherent second trait which was not predicted by the first trait.

### Example 1. Identifying a Selection of ETTIN Homologs

Sequence similarity between major open reading frames of different species can be used to identify uORFs in heterologous genes. AT2G33860 (ETTIN, ARF3, AUXIN RESPONSE TRANSCRIPTION FACTOR 3, SEQ ID NO: 2) encodes an Auxin Response [transcription] Factor that binds to the DNA sequence 5′-TGTCTC-3′ found in auxin-responsive promoter element. The protein sequence of ETTIN from Arabidopsis was used to identify the ETTIN orthologs in various plant species. The sequence was then used in a sequence homology alignment search of the genomes of plant species using BLAST (tblastn) at genomevolution.org/coge/CoGeBlast.pl. In each case the best hit proteins were blasted back against the Arabidopsis proteome. If the top hit from the Arabidopsis proteome was ETTIN with a match with a bit score of greater than 50, the identified crop protein was considered to be an ETTIN homolog. A selection of example ETTIN homologs, identified through this approach, are provided as SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238. Results are shown in Table 1.

### Example 2

The presence of uORFs is determined through application of an algorithm to ribosome profiling data. The algorithm identifies the presence of the uORF based on the existence of ribosome enrichment in the interval from one stop codon to the next stop codon within the same open reading frame,

uORFs of ETTIN homolog-encoding sequences in various plant species were identified through application of an algorithm to ribosome profiling data. The algorithm identifies the presence of the uORF in the genome of an organism based on the existence of ribosome enrichment in the interval from one stop codon to the next stop codon within the same open reading frame. The latter stop codon represents the end of a putative uORF. The sequence immediately upstream of the latter stop codon represents a potential target for gene editing that disrupts the function of the uORF.

Results are shown in Table 1.

TABLE 1
ETTIN-related sequences and potential uORFs found within
the 5′UTR of the gene encoding the ETTIN homolog

| SEQ | | |
|---|---|---|
| ID | | |
| NO: | Description | Plant Genus Species |
| 1 | Arabidopsis ETTIN PROTEIN | Arabidopsis thaliana |
| 2 | AT2G33860.UORF1 | Arabidopsis thaliana |
| 3 | ARF peptide (best hit, like lt 2 other genes close to ARF3) | Citrus × clementina |
| 4 | Potential uORF, lcl\|1:1-87 ORF1 CDS | Citrus × clementina |
| 5 | Potential uORF, lcl\|1:2-187 ORF5 CDS | Citrus × clementina |
| 6 | Potential uORF, lcl\|1:156-386 ORF8 CDS | Citrus × clementina |
| 7 | Potential uORF, lcl\|1:160-249 ORF2 CDS | Citrus × clementina |
| 8 | Potential uORF, lcl\|1:307-411 ORF3 CDS | Citrus × clementina |
| 9 | Potential uORF, lcl\|1:383-571 ORF6 CDS | Citrus × clementina |
| 10 | Potential uORF, lcl\|1:526-621 ORF4 CDS | Citrus × clementina |
| 11 | Potential uORF, lcl\|1:558-749 ORF9 CDS | Citrus × clementina |
| 12 | Potential uORF, lcl\|1:584-676 ORF7 CDS | Citrus × clementina |
| 13 | top hit - gene transcript protein | Prunus avium |
| | Pav_sc0002446.1_g140.1.mk:mrna peptide: | |
| | Pav_sc0002446.1_g140.1.mk:mrna pep:protein_coding | |
| 14 | top hit - gene transcript cDNA 5′UTR | Prunus avium |
| | Pav_sc0002446.1_g140.1.mk:mrna utr5:protein_coding | |
| 15 | Potential uORF, lcl\|1:129-215 ORF4 CDS | Prunus avium |
| 16 | Potential uORF, lcl\|1:216-383 ORF5 CDS | Prunus avium |
| 17 | Potential uORF, lcl\|1:230-406 ORF2 CDS | Prunus avium |
| 18 | Potential uORF, lcl\|1:379-519 ORF1 CDS | Prunus avium |
| 19 | Potential uORF, lcl\|1:407-505 ORF3 CDS | Prunus avium |
| 20 | Potential uORF, lcl\|1:426-518 ORF6 CDS | Prunus avium |
| 21 | top hit - gene transcript protein | Solanum lycopersicum |
| 22 | top hit - gene transcript cDNA 5′UTR Solyc09g007810.3.1 | Solanum lycopersicum |
| | utr5:protein_coding | |
| 23 | Potential uORF lcl\|1:1-114 ORF1 CDS | Solanum lycopersicum |
| 24 | Potential uORF lcl\|1:2-79 ORF5 CDS | Solanum lycopersicum |
| 25 | Potential uORF lcl\|1:147-230 ORF8 CDS | Solanum lycopersicum |
| 26 | Potential uORF lcl\|1:179-283 ORF6 CDS | Solanum lycopersicum |
| 27 | Potential uORF lcl\|1:231-308 ORF9 CDS | Solanum lycopersicum |
| 28 | Potential uORF lcl\|1:238-405 ORF2 CDS | Solanum lycopersicum |
| 29 | Potential uORF lcl\|1:383-466 ORF7 CDS | Solanum lycopersicum |
| 30 | Potential uORF lcl\|1:433-510 ORF3 CDS | Solanum lycopersicum |
| 31 | Potential uORF lcl\|1:511-606 ORF4 CDS | Solanum lycopersicum |
| 32 | Potential uORF lcl\|1:558-671 ORF10 CDS | Solanum lycopersicum |
| 33 | top hit -not annotated second hiy, 3rd Hit Vitvi06g00272_t002 | Vitis vinifera |
| | peptide: Vitvi06g00272_P002 pep:protein coding | |
| 34 | 3rd hit - gene transcript cDNA 5′UTR Vitvi06g00272_t002 | Vitis vinifera |
| | utr5:protein_coding | |
| 35 | Potential uORF lcl\|1:1-102 ORF1 CDS | Vitis vinifera |
| 36 | Potential uORF lcl\|1:15-179 ORF8 CDS | Vitis vinifera |
| 37 | Potential uORF lcl\|1:53-145 ORF5 CDS | Vitis vinifera |
| 38 | Potential uORF lcl\|1:103-216 ORF2 CDS | Vitis vinifera |
| 39 | Potential uORF lcl\|1:146-268 ORF6 CDS | Vitis vinifera |
| 40 | Potential uORF lcl\|1:337-417 ORF3 CDS | Vitis vinifera |
| 41 | Potential uORF lcl\|1:342-455 ORF9 CDS | Vitis vinifera |
| 42 | Potential uORF lcl\|1:401-562 ORF7 CDS | Vitis vinifera |
| 43 | Potential uORF lcl\|1:463-561 ORF4 CDS | Vitis vinifera |
| 44 | top hit - gene transcript protein CDO99747 peptide: CDO99747 | Coffea canephora |
| | pep:protein_coding | |
| 45 | top hit - gene transcript cDNA 5′UTR CDO99747 utr5:protein | Coffea canephora |
| | coding | |
| 46 | Potential uORF lcl\|1:1-105 ORF1 CDS | Coffea canephora |
| 47 | lcl\|1:2-79 ORF4 CDS | Coffea canephora |
| 48 | Potential uORF lcl\|1:3-329 ORF8 CDS | Coffea canephora |
| 49 | Potential uORF lcl\|1:80-157 ORF5 CDS | Coffea canephora |
| 50 | Potential uORF lcl\|1:169-246 ORF2 CDS | Coffea canephora |
| 51 | Potential uORF lcl\|1:227-337 ORF6 CDS | Coffea canephora |
| 52 | Potential uORF lcl\|1:316-525 ORF3 CDS | Coffea canephora |
| 53 | Potential uORF lcl\|1:330-464 ORF9 CDS | Coffea canephora |
| 54 | Potential uORF lcl\|1:386-481 ORF7 CDS | Coffea canephora |
| 55 | top hit gene transcript protein PSS04814 peptide: PSS04814 | Actinidia chinensis |
| | pep:protein_coding | |
| 56 | top hit - gene transcript cDNA 5′UTR PSS04814 utr5:protein | Actinidia chinensis |
| | coding | |
| 57 | Potential uORF lcl\|1:1-81 ORF1 CDS | Actinidia chinensis |
| 58 | Potential uORF lcl\|1:2-184 ORF4 CDS | Actinidia chinensis |
| 59 | Potential uORF lcl\|1:42-119 ORF6 CDS | Actinidia chinensis |
| 60 | Potential uORF lcl\|1:166-333 ORF2 CDS | Actinidia chinensis |
| 61 | Potential uORF lcl\|1:200-349 ORF5 CDS | Actinidia chinensis |
| 62 | Potential uORF lcl\|1:361-513 ORF3 CDS | Actinidia chinensis |
| 63 | Potential uORF lcl\|1:369-500 ORF7 CDS | Actinidia chinensis |
| 64 | top hit - gene transcript protein mRNA:MD11G0104300 peptide: | Malus domestica |
| | mRNA:MD11G0104300 pep:protein coding | |
| 65 | top hit - gene transcript cDNA 5′UTR mRNA:MD11G0104300 | Malus domestica |
| | utr5:protein_coding | |
| 66 | Potential uORF lcl\|1:2-229 ORF4 CDS | Malus domestica |
| 67 | Potential uORF lcl\|1:3-113 ORF6 CDS | Malus domestica |
| 68 | Potential uORF lcl\|1:40-219 ORF1 CDS | Malus domestica |
| 69 | Potential uORF lcl\|1:114-452 ORF7 CDS | Malus domestica |
| 70 | Potential uORF lcl\|1:262-447 ORF2 CDS | Malus domestica |
| 71 | Potential uORF lcl\|1:443-595 ORF5 CDS | Malus domestica |
| 72 | Potential uORF lcl\|1:448-540 ORF3 CDS | Malus domestica |
| 73 | Potential uORF lcl\|1:453-614 ORF8 CDS | Malus domestica |
| 74 | top hit, short 5′UTR, gene transcript protein Cav10g09730.t1 | Corylus avellana |
| | peptide: Cav10g09730.t1 pep:protein coding | |
| 75 | top hit - gene transcript cDNA 5′UTR Cav10g09730.t1 | Corylus avellana |
| | utr5:protein coding | |
| 76 | Potential uORF lcl\|1:1-129 ORF1 CDS | Corylus avellana |
| 77 | Potential uORF lcl\|1:2-148 ORF4 CDS | Corylus avellana |
| 78 | Potential uORF lcl\|1:3-311 ORF6 CDS | Corylus avellana |
| 79 | Potential uORF lcl\|1:130-306 ORF2 CDS | Corylus avellana |
| 80 | Potential uORF lcl\|1:312-500 ORF7 CDS | Corylus avellana |
| 81 | Potential uORF lcl\|1: 362-502 ORF5 CDS | Corylus avellana |
| 82 | Potential uORF lcl\|1:379-501 ORF3 CDS | Corylus avellana |
| 83 | Top hit-gene transcript cDNA 5′UTR Sequence (as a reverse | Persea americana |
| | complement) upstream for the region of complementarity | |
| | gb\|JALDXF010000012.1\|:32100026-32100612 Persea americana | |
| | cultivar Gwen isolate Gwen Chr 12, whole genome shotgun | |
| | sequence | |
| 84 | Potential uORF (−ve strand ORF's as the input sequence was | Persea americana |
| | Reverse complement lc\|1:c81-1 ORF17 CDS | |
| 85 | Potential uORF (−ve strand ORF's as the input sequence was | Persea americana |
| | Reverse complement (−ve strand ORF's as the input sequence was | |
| | Reverse complement lc\|1:c184-2 ORF15 CDS | |
| 86 | Potential uORF (−ve strand ORF's as the input sequence was | Persea americana |
| | Reverse complement lc\|1:c277-200 ORF14 CDS | |
| 87 | Potential uORF (−ve strand ORF's as the input sequence was | Persea americana |
| | Reverse complement lc\|1:c350-264 87 CDS | |
| 88 | Potential uORF (−ve strand ORF's as the input sequence was | Persea americana |
| | Reverse complement lc\|1:c388-278 ORF13 CDS | |
| 89 | Potential uORF (−ve strand ORF's as the input sequence was | Persea americana |
| | Reverse complement lc\|1:c482-369 ORF10 CDS | |
| 90 | Potential uORF (−ve strand ORF's as the input sequence was | Persea americana |
| | Reverse complement lc\|1:c477-385 ORF16 CDS | |
| 91 | Potential uORF (−ve strand ORF's as the input sequence was | Persea americana |
| | Reverse complement lc\|1:c587-483 ORF9 CDS | |
| 92 | Potential uORF (−ve strand ORF's as the input sequence was | Persea americana |
| | Reverse complement lc\|1:c586-506 ORF12 CDS | |
| 93 | Persea americana (v3.0, unmasked), Name: augustus_masked- | Persea americana |
| | Scf00072-processed-gene-2.1-mRNA-1, Type: CDS, Feature | |
| | Location: (Chr: Scf00072, join | |
| | (246378 . . . 247502, 249283 . . . 250175, 250539 . . . 250608)) Genomic | |
| | Location: 246378-25060 | |
| 94 | Top hit-gene transcript cDNA 5′UTR Persea americana (v3.0, | Persea americana |
| | unmasked), Name: augustus_masked-Scf00072-processed-gene- | |
| | 2.1, Type: gene, Feature Location: (Chr: Scf00072, | |
| | 244983 . . . 251293) Genomic Location: 244983-251293 | |
| 95 | Potential uORF lc\|1:2-217 ORF5 CDS | Persea americana |
| 96 | Potential uORF lc\|1:46-285 ORF1 CDS | Persea americana |
| 97 | Potential uORF lc\|1:141-371 ORF13 CDS | Persea americana |
| 98 | Potential uORF lc\|1:218-409 ORF6 CDS | Persea americana |
| 99 | Potential uORF lc\|1:372-473 ORF14 CDS | Persea americana |
| 100 | Potential uORF lc\|1:410-526 ORF7 CDS | Persea americana |
| 101 | Potential uORF lc\|1:484-669 ORF2 CDS | Persea americana |
| 102 | Potential uORF lc\|1:569-658 ORF8 CDS | Persea americana |
| 103 | Potential uORF lc\|1:603-689 ORF15 CDS | Persea americana |
| 104 | Potential uORF lc\|1:690-776 ORF16 CDS | Persea americana |
| 105 | Potential uORF lc\|1:800-90 4ORF9 CDS | Persea americana |
| 106 | Potential uORF lc\|1:882-1046 ORF17 CDS | Persea americana |
| 107 | Potential uORF lc\|1:971-1072 ORF10 CDS | Persea americana |
| 108 | Potential uORF lc\|1:988-1143 ORF3 CDS | Persea americana |
| 109 | Potential uORF lc\|1:1083-1232 ORF18 CDS | Persea americana |
| 110 | Potential uORF lc\|1:1121-1309 ORF11 CDS | Persea americana |
| 111 | Potential uORF lc\|1:1165-1284 ORF4 CDS | Persea americana |
| 112 | Potential uORF lc\|1:1233-1337 ORF19 CDS | Persea americana |
| 113 | Potential uORF lc\|1:1310-1393 ORF12 CDS | Persea americana |
| 114 | AT1G19220.1 \| Symbols: IAA22, ARF19, ARF11 \| AUXIN | Arabidopsis thaliana |
| | RESPONSE FACTOR11, auxin response factor 19, indole-3- | |
| | acetic acid inducible 22 \| chr1: 6628395-6632779 REVERSE | |
| | LENGTH = 1086 | |
| 115 | Predicted uORF AT1G19220.1.1 | Arabidopsis thaliana |
| 116 | Predicted uORF AT1G19220.1.2 | Arabidopsis thaliana |
| 117 | Predicted uORF AT1G19220.1.3 | Arabidopsis thaliana |
| 118 | Predicted uORF AT1G19220.1.4 | Arabidopsis thaliana |
| 119 | Predicted uORF AT1G19220.1.5 | Arabidopsis thaliana |
| 120 | Predicted uORF AT1G19220.1.6 | Arabidopsis thaliana |
| 121 | Predicted uORF AT1G19220.1.7 | Arabidopsis thaliana |
| 122 | Predicted uORF AT1G19220.1.8 | Arabidopsis thaliana |
| 123 | AT1G19850.1 \| Symbols: IAA24, MP, ARF5 \| indole-3-acetic | Arabidopsis thaliana |
| | acid inducible 24, AUXIN RESPONSE FACTOR 5, | |
| | MONOPTEROS \| chr1: 6887353-6891182 FORWARD | |
| | LENGTH = 902 | |
| 124 | Predicted uORF AT1G19850.1.1 | Arabidopsis thaliana |
| 125 | Predicted uORF AT1G19850.1.2 | Arabidopsis thaliana |
| 126 | Predicted uORF AT1G19850.1.3 | Arabidopsis thaliana |
| 127 | Predicted uORF AT1G19850.1.4 | Arabidopsis thaliana |
| 128 | Predicted uORF AT1G19850.1.5 | Arabidopsis thaliana |
| 129 | Predicted uORF AT1G19850.1.6 | Arabidopsis thaliana |
| 130 | Predicted uORF AT1G19850.1.7 | Arabidopsis thaliana |
| 131 | Predicted uORF AT1G19850.1.8 | Arabidopsis thaliana |
| 132 | Predicted uORF AT1G19850.1.9 | Arabidopsis thaliana |
| 133 | Predicted uORF AT1G19850.1.10 | Arabidopsis thaliana |
| 134 | AT1G30330.2 \| Symbols: ARF6 \| auxin response factor 6 \| | Arabidopsis thaliana |
| | chr1: 10686125-10690036 REVERSE LENGTH = 935 | |
| 135 | Predicted uORF AT1G30330.1.1 | Arabidopsis thaliana |
| 136 | Predicted uORF AT1G30330.1.2 | Arabidopsis thaliana |
| 137 | Predicted uORF AT1G30330.1.3 | Arabidopsis thaliana |
| 138 | Predicted uORF AT1G30330.1.4 | Arabidopsis thaliana |
| 139 | Predicted uORF AT1G30330.1.5 | Arabidopsis thaliana |
| 140 | Predicted uORF AT1G30330.1.6 | Arabidopsis thaliana |
| 141 | Predicted uORF AT1G30330.1.7 | Arabidopsis thaliana |
| 142 | Predicted uORF AT1G30330.1.8 | Arabidopsis thaliana |
| 143 | Predicted uORF AT1G30330.1.9 | Arabidopsis thaliana |
| 144 | Predicted uORF AT1G30330.1.10 | Arabidopsis thaliana |
| 145 | Predicted uORF AT1G30330.1.11 | Arabidopsis thaliana |
| 146 | Predicted uORF AT1G30330.1.12 | Arabidopsis thaliana |
| 147 | Predicted uORF AT1G30330.1.13 | Arabidopsis thaliana |
| 148 | Predicted uORF AT1G30330.1.14 | Arabidopsis thaliana |
| 149 | Predicted uORF AT1G30330.1.15 | Arabidopsis thaliana |
| 150 | Predicted uORF AT1G30330.1.16 | Arabidopsis thaliana |
| 151 | Predicted uORF AT1G30330.1.17 | Arabidopsis thaliana |
| 152 | Predicted uORF AT1G30330.1.18 | Arabidopsis thaliana |
| 153 | Predicted uORF AT1G30330.1.19 | Arabidopsis thaliana |
| 154 | AT2G33860.1 \| Symbols: ARF3, ETT \| ETTIN, AUXIN | Arabidopsis thaliana |
| | RESPONSE TRANSCRIPTION FACTOR 3 \| chr2: 14325444- | |
| | 14328613 REVERSE LENGTH = 608 | |
| 155 | AT5G20730.1 \| Symbols: NPH4, MSG1, IAA21, IAA23, ARF7. | Arabidopsis thaliana |
| | IAA25, TIR5, BIP \| BIPOSTO, TRANSPORT INHIBITOR | |
| | RESPONSE 5, MASSUGU 1, indole-3-acetic acid inducible 25, | |
| | AUXIN RESPONSE FACTOR 7, NON-PHOTOTROPHIC | |
| | HYPOCOTYL, indole-3-acetic acid inducible 21, indole-3-acetic | |
| | acid inducible 23 \| chr5: 7016704-7021504 REVERSE | |
| | LENGTH = 1165 | |
| 156 | Predicted uORF AT5G20730.1.1 | Arabidopsis thaliana |
| 157 | Predicted uORF AT5G20730.1.2 | Arabidopsis thaliana |
| 158 | Predicted uORF AT5G20730.1.3 | Arabidopsis thaliana |
| 159 | Predicted uORF AT5G20730.1.4 | Arabidopsis thaliana |
| 160 | Predicted uORF AT5G20730.1.5 | Arabidopsis thaliana |
| 161 | Predicted uORF AT5G20730.1.6 | Arabidopsis thaliana |
| 162 | Predicted uORF AT5G20730.1.7 | Arabidopsis thaliana |
| 163 | Predicted uORF AT5G20730.1.8 | Arabidopsis thaliana |
| 164 | Predicted uORF AT5G20730.1.9 | Arabidopsis thaliana |
| 165 | Predicted uORF AT5G20730.1.10 | Arabidopsis thaliana |
| 166 | Predicted uORF AT5G20730.1.11 | Arabidopsis thaliana |
| 167 | Predicted uORF AT5G20730.1.12 | Arabidopsis thaliana |
| 168 | Predicted uORF AT5G20730.1.13 | Arabidopsis thaliana |
| 169 | AT5G37020.1 \| Symbols: ATARF8, ARF8 \| auxin response factor | Arabidopsis thaliana |
| | 8 \| chr5: 14630151-14634106 FORWARD LENGTH = 811 | |
| 170 | Predicted uORF AT5G37020.1.1 | Arabidopsis thaliana |
| 171 | AT5G60450.1 \| Symbols: ARF4 \| auxin response factor 4 \| | Arabidopsis thaliana |
| | chr5: 24308558-24312187 REVERSE LENGTH = 788 | |
| 172 | Predicted uORF AT5G60450.1.1 | Arabidopsis thaliana |
| 173 | Predicted uORF AT5G60450.1.2 | Arabidopsis thaliana |
| 174 | Predicted uORF AT5G60450.1.3 | Arabidopsis thaliana |
| 175 | Predicted uORF AT5G60450.1.4 | Arabidopsis thaliana |
| 176 | Predicted uORF AT5G60450.1.5 | Arabidopsis thaliana |
| 177 | Predicted uORF AT5G60450.1.6 | Arabidopsis thaliana |
| 178 | Predicted uORF AT5G60450.1.7 | Arabidopsis thaliana |
| 179 | Predicted uORF AT5G60450.1.8 | Arabidopsis thaliana |
| 180 | Predicted uORF AT5G60450.1.9 | Arabidopsis thaliana |
| 181 | Os01g0753500:Os01t0753500-01 peptide: Os01t0753500-01 | Oryza sativa |
| | pep:protein_coding | |
| 182 | Predicted uORF Os01g0753500.1.1 | Oryza sativa |
| 183 | Predicted uORF Os01g0753500.1.2 | Oryza sativa |
| 184 | Predicted uORF Os01g0753500.1.3 | Oryza sativa |
| 185 | Predicted uORF Os01g0753500.1.4 | Oryza sativa |
| 186 | Os05g0515400:Os05t0515400-01 peptide: Os05t0515400-01 | Oryza sativa |
| | pep:protein_coding | |
| 187 | Predicted uORF Os05g0515400.1.1 | Oryza sativa |
| 188 | Os05g0563400:Os05t0563400-01 peptide: Os05t0563400-01 | Oryza sativa |
| | pep:protein_coding | |
| 189 | Predicted uORF Os05g0563400.1.1 | Oryza sativa |
| 190 | Zm00001eb152940:Zm00001eb152940_T001 peptide: | Zea mays |
| | Zm00001eb152940_P001 pep:protein_coding | |
| 191 | Predicted uORF Zm00001eb152940.1.1 | Zea mays |
| 192 | Predicted uORF Zm00001eb152940.1.2 | Zea mays |
| 193 | Predicted uORF Zm00001eb152940.1.3 | Zea mays |
| 194 | Predicted uORF Zm00001eb152940.1.4 | Zea mays |
| 195 | Predicted uORF Zm00001eb152940.1.5 | Zea mays |
| 196 | Predicted uORF Zm00001eb152940.1.6 | Zea mays |
| 197 | Predicted uORF Zm00001eb152940.1.7 | Zea mays |
| 198 | Predicted uORF Zm00001eb152940.1.8 | Zea mays |
| 199 | Zm00001eb157270_T001 peptide: Zm00001eb157270_P001 | Zea mays |
| | pep:protein_coding | |
| 200 | Predicted uORF Zm00001eb157270.1.1 | Zea mays |
| 201 | Zm00001eb292830_T001 peptide: Zm00001eb292830_P001 | Zea mays |
| | pep:protein_coding | |
| 202 | Predicted uORF Zm00001eb292830.1.1 | Zea mays |
| 203 | Predicted uORF Zm00001eb292830.1.2 | Zea mays |
| 204 | Zm00001eb295830_T001 peptide: Zm00001eb295830_P001 | Zea mays |
| | pep:protein_coding | |
| 205 | Predicted uORF Zm00001eb295830.1.1 | Zea mays |
| 206 | Predicted uORF Zm00001eb295830.1.2 | Zea mays |
| 207 | Predicted uORF Zm00001eb295830.1.3 | Zea mays |
| 208 | Predicted uORF Zm00001eb295830.1.4 | Zea mays |
| 209 | Predicted uORF Zm00001eb295830.1.5 | Zea mays |
| 210 | Predicted uORF Zm00001eb295830.1.6 | Zea mays |
| 211 | Predicted uORF Zm00001eb295830.1.7 | Zea mays |
| 212 | Zm00001eb370810_T001 peptide: Zm00001eb370810_P001 | Zea mays |
| | pep:protein_coding | |
| 213 | Predicted uORF Zm00001eb370810.1.1 | Zea mays |
| 214 | Predicted uORF Zm00001eb370810.1.2 | Zea mays |
| 215 | Predicted uORF Zm00001eb370810.1.3 | Zea mays |
| 216 | Predicted uORF Zm00001eb370810.1.4 | Zea mays |
| 217 | Predicted uORF Zm00001eb370810.1.5 | Zea mays |
| 218 | Predicted uORF Zm00001eb370810.1.6 | Zea mays |
| 219 | Predicted uORF Zm00001eb370810.1.7 | Zea mays |
| 220 | Predicted uORF Zm00001eb370810.1.8 | Zea mays |
| 221 | Predicted uORF Zm00001eb370810.1.9 | Zea mays |
| 222 | Predicted uORF Zm00001eb370810.1.10 | Zea mays |
| 223 | Predicted uORF Zm00001eb370810.1.11 | Zea mays |
| 224 | Predicted uORF Zm00001eb370810.1.12 | Zea mays |
| 225 | Predicted uORF Zm00001eb370810.1.13 | Zea mays |
| 226 | Predicted uORF Zm00001eb370810.1.14 | Zea mays |
| 227 | Predicted uORF Zm00001eb370810.1.15 | Zea mays |
| 228 | Predicted uORF Zm00001eb370810.1.16 | Zea mays |
| 229 | Predicted uORF Zm00001eb370810.1.17 | Zea mays |
| 230 | Predicted uORF Zm00001eb370810.1.18 | Zea mays |
| 231 | Predicted uORF Zm00001eb370810.1.19 | Zea mays |
| 232 | Predicted uORF Zm00001eb370810.1.20 | Zea mays |
| 233 | TraesCS1A02G334900.1 peptide: TraesCS1A02G334900.1 | Triticum aestivum |
| | pep:protein_coding | |
| 234 | Predicted uORF TraesCS1A02G334900.1.1 | Triticum aestivum |
| 235 | Predicted uORF TraesCS1A02G334900.1.2 | Triticum aestivum |
| 236 | TraesCS1A02G396400.1 peptide: TraesCS1A02G396400.1 | Triticum aestivum |
| | pep:protein_coding | |
| 237 | Predicted uORF TraesCS1A02G396400.1.1 | Triticum aestivum |
| 238 | TraesCS3A02G292400.1 peptide: TraesCS3A02G292400.1 | Triticum aestivum |
| | pep:protein_coding | |
| 239 | Predicted uORF TraesCS3A02G292400.1.1 | Triticum aestivum |
| 240 | Predicted uORF TraesCS3A02G292400.1.2 | Triticum aestivum |

### Example 3: Selection of a Dwarf Tomato Plant Carrying a Non-Naturally Occurring Allele Comprising a uORF Mutation in an ETTIN Gene

A plant is selected from a tomato mutant population wherein the selected plant contains a mutation in a uORF within the 5′UTR of a tomato ETTIN homolog (SEQ ID NO: 22-32), which is identified by sequencing the genomic region containing the ETTIN homolog from a number of plants from the mutant population and comparing the sequences to the genomic region from a wild-type tomato plant of the same variety. The selected plant is then grown and crossed, either to itself or to a second plant and the progeny seed are then germinated and a plant is selected which exhibit a dwarf phenotype. Optionally, the selected plant may be grafted as a rootstock to a scion from a second plant.

### Example 4: Selection of a Dwarf Tomato Plants with a Non-Naturally Occurring Allele Comprising a Mutation in an ETTIN Gene with the Region Encoding an AUX/IAA Interaction Domain

A plant is selected from a tomato mutant population wherein the selected plant contains a mutation in a region of a tomato ETTIN homolog (SEQ ID NO: 21) that encodes a putative AUX/IAA protein interacting region. The plant is identified by sequencing the genomic region containing the ETTIN homolog from a number of plants from the mutant population and comparing the sequences to the genomic region from a wild-type tomato plant of the same variety. The selected plant is then grown and crossed, either to itself or to a second plant and the progeny seed are then germinated and a plant is selected which exhibit a dwarf phenotype. Optionally, the selected plant may be grafted as a rootstock to a scion from a second plant.

### Example 5: Generation of a Dwarf Apple Plant Harboring a Non-Naturally Carrying a Non-Naturally Allele Comprising a uORF Mutation in an ETTIN Gene

A guide RNA with complementarity to a uORF (SEQ ID NO: 65-73) within the 5′UTR sequence of the gene encoding an apple ETTIN homolog (SEQ ID NO. 64) is introduced into apple cells that expresses a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 6: Generation of a Dwarf Apple Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding an apple ETTIN homolog (SEQ ID NO. 64) is introduced into apple cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 7: Generation of a Dwarf Prunus Plant Harboring a Non-Naturally Carrying a Non-Naturally Allele Comprising a uORF Mutation in an ETTIN Gene

A guide RNA with complementarity to a uORF (e.g., SEQ ID NO: 14-20) within the 5′UTR sequence of the gene encoding an ETTIN homolog from the genus Prunus (e.g., cherry, SEQ ID NO. 13, or plum, cherry, peach, nectarine, apricot, or almond) is introduced into Prunus cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 8: Generation of a Dwarf Prunus Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding an almond ETTIN homolog from the genus Prunus (e.g., cherry, SEQ ID NO. 13, or plum, cherry, peach, nectarine, apricot, or almond) is introduced into Prunus cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 9: Generation of a Dwarf Tomato Plant Tomato Harboring a Non-Naturally Carrying a Non-Naturally Allele Comprising a uORF Mutation in an ETTIN Gene

A guide RNA with complementarity to a uORF (SEQ ID NO: 22-32) within the 5′UTR sequence of the gene encoding a tomato ETTIN homolog (SEQ ID NO. 21) is introduced into tomato cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 10: Generation of a Dwarf Tomato Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a tomato ETTIN homolog (SEQ ID NO. 21) is introduced into tomato cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 11: Generation of a Dwarf Potato Plant Tomato Harboring a Non-Naturally Carrying a Non-Naturally Allele Comprising a uORF Mutation in an ETTIN Gene

A guide RNA with complementarity to a uORF within the 5′UTR sequence of the gene encoding a potato ETTIN homolog is introduced into potato cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 12: Generation of a Dwarf Potato Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a potato ETTIN homolog is introduced into potato cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 13: Generation of a Dwarf Citrus Plant Tomato Harboring a Non-Naturally Carrying a Non-Naturally Allele Comprising a uORF Mutation in an ETTIN Gene

A guide RNA with complementarity to a uORF (SEQ ID NO: 4-12) within the 5′UTR sequence of the gene encoding a citrus ETTIN homolog (SEQ ID NO. 3) is introduced into citrus cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 14: Generation of a Dwarf Citrus Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a citrus ETTIN homolog (SEQ ID NO. 3) is introduced into citrus cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 15: Generation of a Dwarf Avocado Plant Tomato Harboring a Non-Naturally Carrying a Non-Naturally Allele Comprising a uORF Mutation in an ETTIN Gene

A guide RNA with complementarity to a uORF (SEQ ID NO: 83-92, 94-113) within the 5′UTR sequence of the gene encoding an avocado ETTIN homolog (SEQ ID NO. 93) is introduced into avocado cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 16: Generation of a Dwarf Avocado Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding an avocado ETTIN homolog (SEQ ID NO. 93) is introduced into avocado cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 17: Generation of a Dwarf Pistachio Plant Tomato Harboring a Non-Naturally Carrying a Non-Naturally Allele Comprising a uORF Mutation in an ETTIN Gene

A guide RNA with complementarity to a uORF within the 5′UTR sequence of the gene encoding a pistachio ETTIN homolog is introduced into pistachio cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 18: Generation of a Dwarf Pistachio Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a pistachio ETTIN homolog is introduced into pistachio cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 19: Generation of a Dwarf Walnut Plant Tomato Harboring a Non-Naturally Carrying a Non-Naturally Allele Comprising a uORF Mutation in an ETTIN Gene

A guide RNA with complementarity to a uORF within the 5′UTR sequence of the gene encoding a walnut ETTIN homolog is introduced into walnut cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 20: Generation of a Dwarf Walnut Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a walnut ETTIN homolog is introduced into walnut cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 21: Generation of a Dwarf Grape Plant Harboring a Non-Naturally Carrying a Non-Naturally Allele Comprising a uORF Mutation in an ETTIN Gene

A guide RNA with complementarity to a uORF (SEQ ID NO: 34-43) within the 5′UTR sequence of the gene encoding a grape ETTIN homolog (SEQ ID NO. 33) is introduced into grape cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 22: Generation of a Dwarf Grape Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a grape ETTIN homolog (SEQ ID NO. 34) is introduced into grape cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 23: Generation of a Dwarf Kiwifruit Plant Harboring a Non-Naturally Carrying a Non-Naturally Allele Comprising a uORF Mutation in an ETTIN Gene

A guide RNA with complementarity to a uORF (SEQ ID NO: 56-63) within the 5′UTR sequence of the gene encoding a kiwifruit ETTIN homolog (SEQ ID NO. 55) is introduced into kiwifruit cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 24: Generation of a Dwarf Kiwifruit Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a kiwifruit ETTIN homolog (SEQ ID NO. 55) is introduced into kiwifruit cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 25: Generation of a Dwarf Mango Plant Harboring a Non-Naturally Carrying a Non-Naturally Allele Comprising a uORF Mutation in an ETTIN Gene

A guide RNA with complementarity to a uORF within the 5′UTR sequence of the gene encoding a mango ETTIN homolog is introduced into mango cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 26: Generation of a Dwarf Mango Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a mango ETTIN homolog is introduced into mango cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 27: Generation of a Dwarf Plant of a Grain Crop Harboring a Non-Naturally Carrying a Non-Naturally Allele Comprising a uORF Mutation in an ETTIN Gene

A guide RNA with complementarity to a uORF within the 5′UTR sequence of the gene encoding an ETTIN homolog from a grain bearing crop such as rice, wheat, sorghum, or corn is introduced into cells of that crop that express a CAS enzyme and regenerated plants are obtained. A plant is then selected from the regenerated plants, which contains a mutation within the uORF sequence as compared to a wild type plant. The selected plant is then grown and propagated, either sexually or asexually and a progeny plant is selected which exhibits a dwarf phenotype. Optionally, the selected plant may be crossed with a second plant to produce a dwarf hybrid variety.

### Example 28: Generation of a Dwarf Plant of a Grain Crop with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding an ETTIN homolog from a grain bearing crop such as rice, wheat, sorghum, or corn is introduced into cells of that crop that express a CAS enzyme and regenerated plants are obtained. A plant is then selected from the regenerated plants, which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be crossed with a second plant to produce a dwarf hybrid variety.

### Example 29: Generation of a Dwarf Wheat Plant Harboring a Non-Naturally Carrying a Non-Naturally Allele Comprising a uORF Mutation in an ETTIN Gene

A guide RNA with complementarity to a uORF (SEQ ID NO: 234-240) within the 5′UTR sequence of the gene encoding a wheat ETTIN homolog (SEQ ID NO. 233) is introduced into wheat cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 30: Generation of a Wheat Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a wheat ETTIN homolog (SEQ ID NO. 233) is introduced into wheat cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype. Optionally, the selected plant may be grafted as a rootstock to a scion from a second plant.

### Example 31: Generation of a Dwarf Maize Plant Harboring a Non-Naturally Carrying a Non-Naturally Allele Comprising a uORF Mutation in an ETTIN Gene

A guide RNA with complementarity to a uORF (SEQ ID NO: 213-232) within the 5′UTR sequence of the gene encoding a maize ETTIN homolog (SEQ ID NO. 212) is introduced into maize cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype.

### Example 32: Generation of a Maize Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a maize ETTIN homolog (SEQ ID NO. 212) is introduced into maize cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype.

### Example 31: Generation of a Dwarf Rice Plant Harboring a Non-Naturally Carrying a Non-Naturally Allele Comprising a uORF Mutation in an ETTIN Gene

A guide RNA with complementarity to a uORF (SEQ ID NO: 182-189) within the 5′UTR sequence of the gene encoding a rice ETTIN homolog (SEQ ID NO. 181) is introduced into rice cells that express a CAS enzyme and regenerated plants are obtained. A plant is then selected from the regenerated plants which contains a mutation within the uORF sequence as compared to a wild type plant. The selected plant is then grown and propagated, either sexually or asexually and a progeny plant is selected which exhibits a dwarf phenotype.

### Example 32: Generation of a Rice Plant with a Non-Naturally Occurring Allele Comprising a Mutation in an ETTIN Gene with the Region Encoding an AUX/IAA Interaction Domain

A guide RNA with complementarity to the sequence encoding an aux/IAA interaction domain within a gene encoding a rice ETTIN homolog (SEQ ID NO. 181) is introduced into rice cells that express a CAS enzyme and regenerated plants are obtained. A plant is then selected from the regenerated plants which contains a mutation within the aux/iaa interaction domain encoding sequence as compared to a wild type plant. The selected plant is then grown and propagated, either sexually or asexually and a progeny plant is selected which exhibits a dwarf phenotype.

### Example 33: Generation of a Pine Tree with Reduced Branching Harboring a Non-Naturally Carrying a Non-Naturally Allele Comprising a uORF Mutation in an ETTIN Gene

A guide RNA with complementarity to a uORF within the 5′UTR sequence of the gene encoding a pine ETTIN homolog is introduced into pine cells that express a CAS enzyme and regenerated plants are obtained. A plant is then selected from the regenerated plants which contains a mutation within the uORF sequence as compared to a wild type plant, wherein the mutation results in a reduction in levels of the pine ETTIN protein. The selected plant is then grown and propagated, either sexually or asexually and a progeny plant is selected which exhibits a desirable phenotype of reduced branching.

### Example 34: Generation of a Eucalyptus Tree with Reduced Branching Harboring a Non-Naturally Carrying a Non-Naturally Allele Comprising a uORF Mutation in an ETTIN Gene

A guide RNA with complementarity to a uORF within the 5′UTR sequence of the gene encoding a Eucalyptus ETTIN homolog is introduced into Eucalyptus cells that express a CAS enzyme and regenerated plants are obtained. A plant is then selected from the regenerated plants which contains a mutation within the uORF sequence as compared to a wild type plant, wherein the mutation results in a reduction in levels of the Eucalyptus ETTIN protein. The selected plant is then grown and propagated, either sexually or asexually and a progeny plant is selected which exhibits a desirable phenotype of reduced branching.

What is claimed is:

1. A rootstock that is grafted to a scion of an avocado plant, plant part, or plant cell;
wherein the genome of the rootstock harbors a non-naturally occurring allele of a gene that contains a 5′untranslated region (UTR) that lies upstream of a main open reading frame (ORF) polynucleotide sequence;
wherein the ORF encodes an ETTIN homolog polypeptide or a polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to SEQ ID NO: 93, and the 5′UTR contains an upstream open reading frame (uORF) that encodes a uORF-encoded peptide (uPEP) that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a polypeptide encoded by SEQ ID NO: 96; and
wherein the non-naturally occurring allele comprises an introduced mutation in the uORF which regulates expression of the ETTIN homolog polypeptide or a polypeptide, and the rootstock produces a plant that exhibits a dwarf phenotype when grown to maturity.

2. A tomato plant, plant part, or plant cell;
wherein the genome of the tomato plant, plant part, or plant cell harbors a non-naturally occurring allele of a gene that contains a 5′untranslated region (UTR) that lies upstream of a main open reading frame (ORF) polynucleotide sequence;
wherein the ORF encodes an ETTIN homolog polypeptide or a polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to SEQ ID NO: 21, and the 5′UTR contains an upstream open reading frame (uORF) that encodes a uORF-encoded peptide (uPEP) that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a polypeptide encoded by SEQ ID NO: 28; and
wherein the non-naturally occurring allele comprises an introduced mutation in the uORF which regulates expression of the ETTIN homolog polypeptide or a polypeptide, and said regulated expression produces a plant that exhibits a dwarf phenotype.

3. A plant, plant part, or plant cell, the genome of which harbors a non-naturally occurring allele of a gene that contains a 5′untranslated region (UTR) that lies upstream of a main open reading frame (ORF) polynucleotide sequence that encodes an ETTIN homolog polypeptide or a polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, and the 5′UTR contains an upstream open reading frame (uORF) that encodes a uORF-encoded peptide (uPEP) that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a polypeptide encoded by any of SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240, and wherein the allele comprises a mutation in the uORF.

4. The plant part, plant part, or plant cell, of claim 3, wherein the plant part is a rootstock that is grafted to a scion to produce a plant that exhibits a dwarf phenotype when grown to maturity.

5. The plant, plant part, or plant cell of claim 3 or claim 4, wherein the plant is a grain crop, fruit crop, fruit tree, or vine.

6. The plant, plant part, or plant cell of claim 5, wherein the grain crop, fruit crop, fruit tree or vine is selected from the group consisting of; maize, wheat, rice, sorghum, avocado, mango, mangosteen, breadfruit, jackfruit, apple, cherry, plum, pear, peach, nectarine, almond, pistachio, walnut, hazelnut, tomato, kiwi, and citrus.

7. A plant, plant part, or plant cell, the genome of which harbors a non-naturally occurring allele of a gene which when compared to a wild-type allele of the gene, expresses an elevated level of a polypeptide that is an ETTIN homolog polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, and the 5′UTR of the gene contains a uORF that encodes a uPEP that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a polypeptide encoded by any of SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240, and wherein the non-naturally occurring allele comprises a mutation in the uORF and wherein the plant exhibits a dwarf phenotype.

8. The plant part, plant part, or plant cell of claim 7, wherein the plant part is a root stock that is grafted to a scion to produce a plant that exhibits a dwarf phenotype when grown to maturity.

9. The plant, plant part, or plant cell of claim 7 or claim 8, wherein the plant is a grain crop, fruit crop, fruit tree, or vine.

10. The plant, plant part, or plant cell of claim 9, wherein the grain crop, fruit crop, fruit tree, or vine is selected from the group consisting of; maize, rice, wheat, sorghum, avocado, mango, mangosteen, breadfruit, jackfruit, apple, cherry, plum, pear, peach, nectarine, almond, pistachio, walnut, hazelnut, tomato, kiwi, and citrus.

11. The non-naturally occurring allele of claim 3 or 7 wherein the allele is produced through gene editing or use of a Cas enzyme.

12. The plant, plant part, or plant cell of claim 1 or claim 5 wherein the allele is selected from a mutated population using the technique of TILLING.

13. The plant of claim 1 or claim 5 wherein the uORF is at least 70%, 71%, 72%, 73%, 74%, 75%, 76%, 77%, 78%, 79%, 80%, 81%, 82%, 83%, 84%, 85%, 86%, 87%, 88%, 89%, 90%, at least 91%, at least 92%, at least 93%, at least 94%, at least 95%, at least 96%, at least 97%, at least 98%, at least 99% or about 100% to a sequence from the group: SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240.

14. A method of producing a population of dwarf plants comprising crossing a plant of claim 3 or claim 7 to a second plant and growing seed from the descendants of a resulting cross.

15. A method of producing new plant variety comprising introducing or selecting a mutation in a cell of the plant species wherein the mutation is in a uORF sequence within the 5′UTR of gene that encodes a polypeptide that is an ETTIN homolog or a polypeptide with at least 30%, at least 40%, at least 50%, at least 60%, at least 70%, at least 75%, at least 80%, at least 85%, at least 90%, at least 91%, at least 92%, at least 93%, at least 96%, at least 97%, at least 98%, at least 99% or 100% identity to any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, and wherein the cell is regenerated into a plant or plant part which is selected for an increased level of the polypeptide and the presence of a dwarf phenotype, and then multiplying the selected plant or plant part through a process of vegetative propagation, grafting or tissue culture or crossing.

16. The method of claim 15, wherein the uORF is at 70%, 71%, 72%, 73%, 74%, 75%, 76%, 77%, 78%, 79%, 80%, 81%, 82%, 83%, 84%, 85%, 86%, 87%, 88%, 89%, 90%, at least 91%, at least 92%, at least 93%, at least 94%, at least 95%, at least 96%, at least 97%, at least 98%, at least 99% or about 100% to a sequence from SEQ ID NO: 42, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240.

17. A gene-editing construct comprising a polynucleotide sequence encoding a gene editing or DNA repair enzyme, and at least one guide RNA sequence targeting a target site in the 5′UTR of gene that contains a uORF that encodes a uPEP that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to a polypeptide encoded by any of SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240; wherein the construct alters the expression level or activity of a polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238.

18. The gene-editing construct of claim 17, wherein the gene-editing construct is a CRISPR/Cas9 construct.

19. The plant of claim 17, wherein the plant exhibits a dwarf phenotype or at least one dwarfing-associated attribute selected from the group consisting of a) altered auxin transport, b) slower auxin transport, c) reduced apical dominance, d) an altered xylem/phloem ratio, e) an increased number of phloem elements, f) smaller phloem elements, g) thicker bark, h) a bushier habit, i) reduced root mass, and j) reduced vigor, k) less vegetative growth, 1) earlier termination of shoot growth, m) earlier competence to flower, n) precocity, o) earlier phase change, p) smaller canopy, q) reduced stem circumference, r) reduced branch diameter, s) fewer sylleptic branches, t) shorter sylleptic branches, u) more axillary flowers, v) an earlier terminating primary axis, w) earlier terminating secondary axes, x) shorter internode length and y) reduced scion mass.

20. The construct of claim 17, wherein the guide RNA sequence is complementary to a uORF comprising SEQ ID NO: 2, 4-12, 14-20, 22-32, 34-43, 45-54, 56-63, 65-73, 75-92, 94-113, 115-122, 124-133, 135-153, 156-168, 170, 172-180, 182-185, 187, 189, 191-198, 200, 202-203, 205-211, 213-232, 234-235, 237, or 239-240.

21. A host cell comprising the construct of claim 17.

22. The host cell of claim 21, wherein the host cell is a bacterial cell.

23. The host cell of claim 21, wherein the host is an Agrobacterium.

24. A plant tissue comprising the host cell of claim 21.

25. The plant tissue of claim 24, wherein the tissue is a root tissue.

26. A method of producing a plant with reduced expression level of an ETTIN homolog polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, the method comprising expressing the construct of claim 17 in a plant cell wherein the plant cell is from shrub or tree, and selecting a regenerated plant that shows a reduction in branching or apical dominance compared to a control plant.

27. The method of claim 26, wherein said reduced expression level of an ETTIN homolog polypeptide is produced by a gene edit of a uORF that alters a non-canonical start codon into an ATG start codon.

28. The method of claim 26, wherein the expression level of an ETTIN homolog polypeptide is reduced to a level of about 0%, or to about or at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, or 99%, or to 0% to 10%, or to or to 10% to 20%, or to 20% to 30%, or to 30% to 40%, or to 40% to 50%, or to 50% to 60%, or to 60% to 70%, or to 70% to 80%, or to 80% to 90%, or to 90% to 99%, of the expression level of the ETTIN homolog polypeptide in a control plant.

29. A method of increasing the production of an active protein from a polynucleotide in a plant cell, the method comprising introducing or selecting a sequence change within a uORF that resides upstream of the start codon of the main ORF that encodes the active protein and wherein the uORF sequence was first identified in the 5′UTR of a gene that encodes a protein that is an ETTIN homolog or a protein that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% percent identity to a SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238.

30. A plant regenerated from the plant cell with increased production of an active protein produced by the method of claim 29.

31. The method of claim 29, herein the active protein is luciferase, renilla, or a fluorescent protein such as GFP.

32. A plant, plant part, or plant cell, the genome of which harbors a non-naturally occurring allele of a gene which when compared to a wild-type allele of the gene, expresses a reduced level of a polypeptide that is an ETTIN homolog polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 96%, 97%, 98%, 99% or about 100% identical to any of SEQ ID NO: 1, 3, 13, 21, 33, 44, 55, 64, 74, 93, 114, 123, 134, 154, 155, 169, 171, 181, 186, 188, 190, 199, 201, 204, 212, 233, 236, or 238, and wherein the 5′UTR of the gene contains a uORF, and wherein the non-naturally occurring allele comprises a mutation in the uORF, and wherein the plant, plant part or plant cell is a species of shrub or tree.

33. The tree or shrub of claim 32, wherein the tree or shrub is a plant of the Pinus, Salix, Eucalyptus, Betula, or Acacia genera.


SEQUENCE LISTING (240 sequences)

SEQ ID NO: 1 - protein, 608 aa, Arabidopsis thaliana
MGGLIDLNVMETEEDETQTQTPSSASGSVSPTSSSSASVSVVSSNSAGGGVCLELWHACAGPLISLPKRGSLVLYFPQGHLEQAPDFSAAIYGLPPHVFCRILDVKLHAETTTDEVYAQVSLLPESEDIERKVREGIIDVDGGEEDYEVLKRSNTPHMFCKTLTASDTSTHGGFSVPRRAAEDCFPPLDYSQPRPSQELLARDLHGLEWRFRHIYRGQPRRHLLTTGWSAFVNKKKLVSGDAVLFLRGDDGKLRLGVRRASQIEGTAALSAQYNQNMNHNNFSEVAHAISTHSVFSISYNPKASWSNFIIPAPKFLKVVDYPFCIGMRFKARVESEDASERRSPGIISGISDLDPIRWPGSKWRCLLVRWDDIVANGHQQRVSPWEIEPSGSISNSGSFVTTGPKRSRIGFSSGKPDIPVSEGIRATDFEESLRFQRVLQGQEIFPGFINTCSDGGAGARRGRFKGTEFGDSYGFHKVLQGQETVPAYSITDHRQQHGLSQRNIWCGPFQNFSTRILPPSVSSSPSSVLLTNSNSPNGRLEDHHGGSGRCRLFGFPLTDETTAVASATAVPCVEGNSMKGASAVQSNHHHSQGRDIYAMRDMLLDIAL

SEQ ID NO: 2 - DNA, 279 bp, Arabidopsis thaliana
  source 1..279: mol_type = genomic DNA
atgtctctctctccacgcacaaaaggtctaaaagccacaccacacacatcagtcaccagacgtagcagagagcctcactgttgcagagagcactcagtactgttctgtttctctgatacctctctctctcctctctcttttaacattgtccaaattaaaaatctaaactttttttctagttttttttttttctttaatagaaaagttttttttctccacggcttaaagactcactcatcactgtgctactactctctcttcttttggctgagagggtaa

SEQ ID NO: 3 - protein, 703 aa, Citrus x clementina
MITFMDSKEKLKEVEKCLDSQLWHACAGGMVQMPPVNSRVFYFPQGHAEHACGPVDFRSCRRIPPYILCRVSAIKFMADPETDEVYAKIKLVPVSNNDPDFDNDDGIAGIHSNETQDKPASFAKTLTQSDANNGGGFSVPRYCAETIFPRLDYSADPPVQTILAKDVHGETWKFRHIYRGTPRRHLLTTGWSTFVNHKKLVAGDSIVFLRAENGDLCVGIRRAKRGIGGGPEVTSGWNGNCVTPYGGFSAFLREDDNKLMRNGIGNVNGPSSNNGNNLMAKGKVRPESVIEAATLAANRQPFEVVYHPRASTPEFCVKASMVKAALQIRWCSGMRFKMAFETEDSSRISWFMGTISSVQVSDPLYWPDSPWRLLQVTWDEPDLLQNVKRVSPWLVELVSNMPTIHLSPFSPPRKKSRLPQPPDFPLDGQLPMPSFSGSLLGPNSPFGCLPDNTPAGMQGARHAHYGLSLSDLHLNKLQSGLSPAGFPPFDRAAKPTRASNSPILQKPSMSENISCLLTMSHSTQPSKKADDLKTPQLVLFGKPILTEQQMSHSCSGDTVSPVRTGNSSSEGNLDKLTNFSDGSGSALQQQGLPDRSFCEVFQWYKDNRQETEPSLETGHCKVFMESEDVGRTLDLSLLGSYDELYKKLAEMFGIENAETLSHLLYRDVTGAVKHIGDEPFSDFMKTARRLTILMDSGSDNVGV

SEQ ID NO: 4 - DNA, 87 bp, Citrus x clementina
  source 1..87: mol_type = genomic DNA
gctttcttcagatccgaagaaaaaggtccccaaaaaaataaaaatcacacagagtactggtggtttcagtactattgcacaacttga

SEQ ID NO: 5 - DNA, 186 bp, Citrus x clementina
  source 1..186: mol_type = genomic DNA
ctttcttcagatccgaagaaaaaggtccccaaaaaaataaaaatcacacagagtactggtggtttcagtactattgcacaacttgatattttcttcctgttttctctgttaaataaattaagggggaaaaaaaaagcctatttagtttctttaagtgaagtgcgtgccgtttgggctcttgcatga

SEQ ID NO: 6 - DNA, 231 bp, Citrus x clementina
  source 1..231: mol_type = genomic DNA
gtgaagtgcgtgccgtttgggctcttgcatgatccgaaaaggtgttccctttgtttctttctttctctgtgtgaaagtgtgtgttttgccttaatctgcttttgtgtcggcctttttaactcaaagactcatcacccagttcaaagtgtaattagatctttgtataggtgtaataaatctctgtatcagtttttttcttttcctttttccttttgtttttggattgattga

SEQ ID NO: 7 - DNA, 90 bp, Citrus x clementina
  source 1..90: mol_type = genomic DNA
agtgcgtgccgtttgggctcttgcatgatccgaaaaggtgttccctttgtttctttctttctctgtgtgaaagtgtgtgttttgccttaa

SEQ ID NO: 8 - DNA, 105 bp, Citrus x clementina
  source 1..105: mol_type = genomic DNA
ttagatctttgtataggtgtaataaatctctgtatcagtttttttcttttcctttttccttttgtttttggattgattgagcagaaaccaagattttttttttaa

SEQ ID NO: 9 - DNA, 189 bp, Citrus x clementina
  source 1..189: mol_type = genomic DNA
ttgagcagaaaccaagattttttttttaatttattttggtggctttcgagttatggagctgttttgaagaaagttgaagcttttttcttgctaagggctgaattggaagtaaatttttggtttgcaatgttttatttttatgaatttttggtgtgcaagtggagatatggcataaaggttcattattga

SEQ ID NO: 10 - DNA, 96 bp, Citrus x clementina
  source 1..96: mol_type = genomic DNA
atttttggtgtgcaagtggagatatggcataaaggttcattattgatagaaggtatgaaattccacttcaaattgtttgtaccatcacttcactga

SEQ ID NO: 11 - DNA, 192 bp, Citrus x clementina
  source 1..192: mol_type = genomic DNA
aggttcattattgatagaaggtatgaaattccacttcaaattgtttgtaccatcacttcactgatattattctttgtactttatttttctgttttcaattttgtgaatatagtgtctaatttggtaatctattcggtttttttaacagagataaacattaagaagaaggggcttatttcttttatgaatttg

SEQ ID NO: 12 - DNA, 93 bp, Citrus x clementina
  source 1..93: mol_type = genomic DNA
aattccacttcaaattgtttgtaccatcacttcactgatattattctttgtactttatttttctgttttcaattttgtgaatatagtgtctaa

SEQ ID NO: 13 - protein, 719 aa, Prunus avium
MRDSERSLDPQLWHACAGGMVHMPPVNSKVFYFPQGHAEHAQAHVDFSSASLRIPSLILCRVASIKFMADPETDEVFAKIKLNPIENNEPYASDEGSMEADGSENQEKPASFAKTLTQSDANNGGGFSVPRYCAETIFPRLDYSADPPVQTVIAKDVHGEIWKFRHIYRGTPRRHLLTTGWSTFVNQKKLVAGDSIVFLRAENGDLCVGIRRAKRGLDGAGGPELASGWNNSHGGGSGACVLPYGGFSVFLREEENKMVRNGGGNLSPNGNLRGKGRVRPESVVEAATLAANGQAFEVVYYPRASTPEFCVKASAVRAAMRVQWCSGMRFKMAFETEDSSRISWFMGTIASVQVSDPIRWPNSPWRLLQVTWDEPDLLQNVKCVNPWLIELVSNFPMIHMSPFSPPRKKLRLPQPDFTLDGQLTLPSFSGNPLGPSSPLCCLPDNTTAGIQGARHAQFRISLSDLHVNNKLQSGLFQSSFQRCDQNSRISNGIRAGHTSSNENLSCLLTMGNSSQNSEKSDNVKKHQFLLFGQPILTEQQISRSCSSDAVSQLLTGKDSKDGNQDRTKFLSDGSKSALEPQVSLEKSSNAGFPWNKDFQGSDFELDTGHCKVFMESEDVGRTLDLSVLGSYEELYRRLANMFGIEKPGLLSHVLYRDVTGAVKQTGVEPFSDFMKKAKRLTILTHPIGESIGRTWIRGMQNAENGLSATNKTGPLSIFA

SEQ ID NO: 14 - DNA, 520 bp, Prunus avium
  source 1..520: mol_type = genomic DNA
ggaaaaattcaggtgagagaagagagagagagggagctgatcttattaaagcttggagctttttaaagtaatggagctaattcacagtgagaaaaagcagatggctactgggtagtgagagaatctaaacagtcattctctctctgattcctgtgtgttacattgaagtgaaacaagaagaagggaaggggctaaacaagcaaagacagagctgaaagaattggagtaaaaaagtgaaccccacacgcactcgacactcctattcggtgcttttcttttttgttgtgacgattttgttgtacacgagcatttactcactgaatcaccaaaagctctctcaaaaaggtgcaaagctttttcccttttcttcttcgttaacatgaagttgaagcctttattgccttgaaccaacagcaacattctaacaacaacaacaacaaccaaaaccagaaaacaaaagaacccaaaaacaatcactgtttttcatcatccggataccttttgaaattgactcaaaacc

SEQ ID NO: 15 - DNA, 87 bp, Prunus avium
  source 1..87: mol_type = genomic DNA
acagtcattctctctctgattcctgtgtgttacattgaagtgaaacaagaagaagggaaggggctaaacaagcaaagacagagctga

SEQ ID NO: 16 - DNA, 168 bp, Prunus avium
  source 1..168: mol_type = genomic DNA
aagaattggagtaaaaaagtgaaccccacacgcactcgacactcctattcggtgcttttcttttttgttgtgacgattttgttgtacacgagcatttactcactgaatcaccaaaagctctctcaaaaaggtgcaaagctttttcccttttcttcttcgttaacatga

SEQ ID NO: 17 - DNA, 177 bp, Prunus avium
  source 1..177: mol_type = genomic DNA
aaaagtgaaccccacacgcactcgacactcctattcggtgcttttcttttttgttgtgacgattttgttgtacacgagcatttactcactgaatcaccaaaagctctctcaaaaaggtgcaaagctttttcccttttcttcttcgttaacatgaagttgaagcctttattgccttga

SEQ ID NO: 18 - DNA, 141 bp, Prunus avium
  source 1..141: mol_type = genomic DNA
catgaagttgaagcctttattgccttgaaccaacagcaacattctaacaacaacaacaacaaccaaaaccagaaaacaaaagaacccaaaaacaatcactgtttttcatcatccggataccttttgaaattgactcaaaac

SEQ ID NO: 19 - DNA, 99 bp, Prunus avium
  source 1..99: mol_type = genomic DNA
accaacagcaacattctaacaacaacaacaacaaccaaaaccagaaaacaaaagaacccaaaaacaatcactgtttttcatcatccggataccttttga

SEQ ID NO: 20 - DNA, 93 bp, Prunus avium
  source 1..93: mol_type = genomic DNA
caacaacaacaacaaccaaaaccagaaaacaaaagaacccaaaaacaatcactgtttttcatcatccggataccttttgaaattgactcaaaa

SEQ ID NO: 21 - protein, 718 aa, Solanum lycopersicum
MITFMDPKEKVKEVEKCLDSQLWHACAGSMVQMPSISSKVFYFPQGHSEHASGNVDFRSSIRIPSYIPCKVSAIKYMADPETDEVFAKIRLIPVGRNEVEFDDDGVVGMNGSDNQDKPSSFAKTLTQSDANNGGGFSVPRYCAETIFPRLDYSADPPVQTILAKDVHGETWKFRHIYRGTPRRHLLTTGWSTFVNHKKLVAGDSIVFLRAENGDLCVGIRRAKRGIGGGPETSSGWNPAGGNCMVPYGGFSSFLREDENKLMRNGNGNNGGNLMNKGKVKAESVIEAANLAASGQPFEVIYYPRASTPEFCVKSSLVKSALQIRWCSGMRFKMPFETEDSSRISWFMGTISSVQVSDPIRWPDSPWRLLQVTWDEPDLLQNVKRVSPWLVELVSNMPTIHLSPFSPPRKKLRLPQHPDFPLDGHLPMPAFSGNHLLGPNSPFGCLPDNTPAGMQGARHAQYGLSLSDLHFNKLHSSLFPVGFPPLDQAAAAPRRPLNSPMISKPCNNENISCLLTMGNSAHSTKKSDIGKAPQLVLFGQPILTEQQISLSCSGDTVSTVRTGNSSSDGNADKIGNVSDGSGSALNQRGLTERSPCDTFQSDPNTEIGHCKVFMESEDVGRTLDLSLLGSYEELCRKLANMFGIDNSEMLNHVLYRDTTGSVKQLGDEPFRYVLKRFPILHYLFIHIMNISRYFRCKEVFLSSILQRLHENSAKVNNSN

SEQ ID NO: 22 - DNA, 678 bp, Solanum lycopersicum
  source 1..678: mol_type = genomic DNA
atcatcatatatatgtatacatctttttccattttgtatgatacaaacagagagctctctctttctcttttgtgtgtgaaatatatatttgtgtattttgagacaaaaacatgacaaagagtattgttagtgaaagcccactctaaaccagtttttcactttccatcaattttgatgatttgttaggtttttcttgggtttttttcttgtttttgtttctgtctggttgaattctgaagtggggttctgttattttgctttgggtgtgggaaaaacagtgtaaaggtggaatcttttggtttctttgagcaactttttgagtgtttcttgatttttgagaagaaaagattggatttttggcttgattttgagctcaaagtgagtcaaggttgctggagaacttagtaattttgtgtttaaatggatgattaggagggaattttatgttttgaagtggtcaaattgaagaaaatgaggagttttgggttgaatcttgtaaaattgttctaatttggtggtaattcaacaaattctcttgagttattatgtacttataattggtgtttctggataaaaaaagcatttacattcctaatgggaaaataagtatttatgaggtgaaagaattgttggttcttgaattgattgttgtaatagtaagattttgttgacatcaag

SEQ ID NO: 23 - DNA, 114 bp, Solanum lycopersicum
  source 1..114: mol_type = genomic DNA
atcatcatatatatgtatacatctttttccattttgtatgatacaaacagagagctctctctttctcttttgtgtgtgaaatatatatttgtgtattttgagacaaaaacatga

SEQ ID NO: 24 - DNA, 78 bp, Solanum lycopersicum
  source 1..78: mol_type = genomic DNA
tcatcatatatatgtatacatctttttccattttgtatgatacaaacagagagctctctctttctcttttgtgtgtga

SEQ ID NO: 25 - DNA, 84 bp, Solanum lycopersicum
  source 1..84: mol_type = genomic DNA
accagtttttcactttccatcaattttgatgatttgttaggtttttcttgggtttttttcttgtttttgtttctgtctggttga

SEQ ID NO: 26 - DNA, 105 bp, Solanum lycopersicum
  source 1..105: mol_type = genomic DNA
tttgttaggtttttcttgggtttttttcttgtttttgtttctgtctggttgaattctgaagtggggttctgttattttgctttgggtgtgggaaaaacagtgtaa

SEQ ID NO: 27 - DNA, 78 bp, Solanum lycopersicum
  source 1..78: mol_type = genomic DNA
attctgaagtggggttctgttattttgctttgggtgtgggaaaaacagtgtaaaggtggaatcttttggtttctttga

SEQ ID NO: 28 - DNA, 168 bp, Solanum lycopersicum
  source 1..168: mol_type = genomic DNA
agtggggttctgttattttgctttgggtgtgggaaaaacagtgtaaaggtggaatcttttggtttctttgagcaactttttgagtgtttcttgatttttgagaagaaaagattggatttttggcttgattttgagctcaaagtgagtcaaggttgctggagaacttag

SEQ ID NO: 29 - DNA, 84 bp, Solanum lycopersicum
  source 1..84: mol_type = genomic DNA
gtcaaggttgctggagaacttagtaattttgtgtttaaatggatgattaggagggaattttatgttttgaagtggtcaaattga

SEQ ID NO: 30 - DNA, 78 bp, Solanum lycopersicum
  source 1..78: mol_type = genomic DNA
gagggaattttatgttttgaagtggtcaaattgaagaaaatgaggagttttgggttgaatcttgtaaaattgttctaa

SEQ ID NO: 31 - DNA, 96 bp, Solanum lycopersicum
  source 1..96: mol_type = genomic DNA
tttggtggtaattcaacaaattctcttgagttattatgtacttataattggtgtttctggataaaaaaagcatttacattcctaatgggaaaataa

SEQ ID NO: 32 - DNA, 114 bp, Solanum lycopersicum
  source 1..114: mol_type = genomic DNA
ttggtgtttctggataaaaaaagcatttacattcctaatgggaaaataagtatttatgaggtgaaagaattgttggttcttgaattgattgttgtaatagtaagattttgttga

SEQ ID NO: 33 - protein, 711 aa, Vitis vinifera
MKETEKSLDSQLWHACAGGMVQMPLVSSKVFYFPQGHAEHAHTNVDFAAAPRIPALVLCRVAAVKFMADPETDEVYAKIRLVPIANNELDCEDDGVMGSSGSEAPEKPASFAKTLTQSDANNGGGFSVPRYCAETIFPRLDYSADPPVQTVIAKDVHGEIWKFRHIYRGTPRRHLLTTGWSTFVNQKKLVAGDSIVFLRAENGDLCVGIRRAKRGIAGGPESPSGWNPASPYAGFPKFLREDESKLMRNGVGGNSNPGSGGGLRGGRRVRPESVVEAATLAANGQPFEVVYYPRASTPEFCVKASGVRSAVRIQWCSGMRFKMPFETEDSSRISWFMGTISSVQVADPIRWPNSPWRLLQVTWDEPDLLQNVKRVSPWLVELVSNMPIIHLSPFSPPRKKLRIPQHPDFPFDGQFPMSSFSSNPLGSSSPLCCLPDNTPAGIQGARHAQYGISLSDLHLNNKLQSGLFPTSFQRFDQHSRITNGIITAHRKNNESISCLLTMGNSSQNLEKSANEKTPQFLLFGQPILTEQQMSRTCSSDAVSQVLTGKSSSDGSSDKAKKISDGLGSTLEQKGKPENLSNVGFSWHQGFQTTEIGLDTGHCKVFMESEDVGRSLDLSVLGSYEELYTRLANMFGIERSETFSHVLYRDATGAVKHTGDEPFSDFTKKAKRLTILMDSGSNNIGRTWITGMRNAENGLDSSNKTGPLSIFA

SEQ ID NO: 34 - DNA, 562 bp, Vitis vinifera
  source 1..562: mol_type = genomic DNA
ggctccggttgtagagagagagttggattttcagatctgaggaaaaagataagaagaaaagggaagacaagaaaagaaaagaaaatcaggcaagggagttgatccatttttttttttgcttttccttcgttggtttggagtctagcgatggaagaaggcatgggatggatttatattaatctctgcttctcaatcagattgtgtatcgttcgctgagaaaataggagtaagtgaaggaataatttttctatcttgtttttcaggttgagagttgaggttcccgatttgaatcatcgattttgaaccatttgggtagttgaatctgaagttttttgagttgaaggagtgtgcagatttgtagtgaatgtaaggagcgtcaaccgtgggagagtgttgttgaagagaggggaactgtgagggagggctgcgactgtggttgttgtttgagcatttaagctatgaggaagaggttgttgttgtggaggttattgctctgaaaagcgctgttgtgacaagtgttttcgttttgaagttcagaaatcgggatttttgttaccggata

SEQ ID NO: 35 - DNA, 102 bp, Vitis vinifera
  source 1..102: mol_type = genomic DNA
ggctccggttgtagagagagagttggattttcagatctgaggaaaaagataagaagaaaagggaagacaagaaaagaaaagaaaatcaggcaagggagttga

SEQ ID NO: 36 - DNA, 165 bp, Vitis vinifera
  source 1..165: mol_type = genomic DNA
agagagagttggattttcagatctgaggaaaaagataagaagaaaagggaagacaagaaaagaaaagaaaatcaggcaagggagttgatccatttttttttttgcttttccttcgttggtttggagtctagcgatggaagaaggcatgggatggatttatattaa

SEQ ID NO: 37 - DNA, 93 bp, Vitis vinifera
  source 1..93: mol_type = genomic DNA
gaagaaaagggaagacaagaaaagaaaagaaaatcaggcaagggagttgatccatttttttttttgcttttccttcgttggtttggagtctag

SEQ ID NO: 38 - DNA, 114 bp, Vitis vinifera
  source 1..114: mol_type = genomic DNA
tccatttttttttttgcttttccttcgttggtttggagtctagcgatggaagaaggcatgggatggatttatattaatctctgcttctcaatcagattgtgtatcgttcgctga

SEQ ID NO: 39 - DNA, 123 bp, Vitis vinifera
  source 1..123: mol_type = genomic DNA
cgatggaagaaggcatgggatggatttatattaatctctgcttctcaatcagattgtgtatcgttcgctgagaaaataggagtaagtgaaggaataatttttctatcttgtttttcaggttga

SEQ ID NO: 40 - DNA, 81 bp, Vitis vinifera
  source 1..81: mol_type = genomic DNA
gttgaaggagtgtgcagatttgtagtgaatgtaaggagcgtcaaccgtgggagagtgttgttgaagagaggggaactgtga

SEQ ID NO: 41 - DNA, 114 bp, Vitis vinifera
  source 1..114: mol_type = genomic DNA
aggagtgtgcagatttgtagtgaatgtaaggagcgtcaaccgtgggagagtgttgttgaagagaggggaactgtgagggagggctgcgactgtggttgttgtttgagcatttaa

SEQ ID NO: 42 - DNA, 162 bp, Vitis vinifera
  source 1..162: mol_type = genomic DNA
agagaggggaactgtgagggagggctgcgactgtggttgttgtttgagcatttaagctatgaggaagaggttgttgttgtggaggttattgctctgaaaagcgctgttgtgacaagtgttttcgttttgaagttcagaaatcgggatttttgttaccggata

SEQ ID NO: 43 - DNA, 99 bp, Vitis vinifera
  source 1..99: mol_type = genomic DNA
ggaagaggttgttgttgtggaggttattgctctgaaaagcgctgttgtgacaagtgttttcgttttgaagttcagaaatcgggatttttgttaccggat

SEQ ID NO: 44 - protein, 668 aa, Coffea canephora
MNSGNEPMNEVEKSLDPQLWHACAGGMVQMSPVNSKVFYFPQGHAEHAHKSVDFGTFCRIPPLILCRVSSIKYLADTETDEVFAKIRLAPLRGNECSDDDGDDGLLAFDKNDGQEKPSSFAKTLTQSDANNGGGFSVPRYCAETIFPRLDYSAEPPVQTILAKDVHGEIWKFRHIYRGTPRRHLLTTGWSNFVNQKKLVAGDSIVFLRAENGDLCVGIRRAKRGIGGEEENRIMRNPKGGTNNSDIGARGRGKVRAESVVEAANLAASGQAFEVIYYPRASTPEFVVKASAVKAAIRIQWCSGMRFKMPFETEDSSRISWFMGTVSSVQVDDPIHWPNSPWRLLQVAWDEPDLLQNVKRVSPWLVELVSNMPAINLSPFSPPRKKLRLPQPPEFPLVGQLPMPSLFSNPLSPSSPLCCLPDKIPAGIQGARHAQFGLPSSEPHFNKLQAGLFPFKLKQLEHAAAAASRIPNSSCFMEDYESKDNVSCVLTIGNSVQGSKPNVRTEAPLFVLFGQPILTEQQISQSSSGDTARSSLSERNPENTVTVSGGSGSGVLQSGHPENSLDQVLPWYKDPKLEFGLETGHCKVFMESEDVGRTLDLSVFGSYEELYGKLAEMFGLERSEMLSNVLYQDPAGVVKHSGDEPFSDFLKAARRITILTDSGSDNVGR

SEQ ID NO: 45 - DNA, 610 bp, Coffea canephora
  source 1..610: mol_type = genomic DNA
ctggagtccggactgctgctgctgctatcatcagtcgtgtttctcctgtcaaaagaaatttacatcacccccacaataatcttcaccctcttgtttttcctttgaatcaatacaaactagtgaaaaaggttggccttttttttttttttttctttaaagttcccttgagagcaaaaaagagagaatggaacatcagtttgtcaaaaagccagtatcttgaaggtaactcaagatgccttgttctagtatttctcccgactctctctctatatatccccctcacccttttcttcagttttttttcctttggattgattgctggggactgaagggttgaatccatgtattttgcctatttttatatgggttgtttcaacaacattaaaaatgtatcttcgtcttcgcaattgcagtttattttcttccagtattttactctcgtcctgcaatccctttgtgtttaaagtacttcagatcctaatacttgtaatcttgttgtcttgtttcagagatttgattgattgagaaaaactaggacccagttaaaggattgcttgtgttgtgtatgcgtttgaggccttagatttgcaaaagaagaatgattactgtg

SEQ ID NO: 46 - DNA, 105 bp, Coffea canephora
  source 1..105: mol_type = genomic DNA
ctggagtccggactgctgctgctgctatcatcagtcgtgtttctcctgtcaaaagaaatttacatcacccccacaataatcttcaccctcttgtttttcctttga

SEQ ID NO: 47 - DNA, 78 bp, Coffea canephora
  source 1..78: mol_type = genomic DNA
tggagtccggactgctgctgctgctatcatcagtcgtgtttctcctgtcaaaagaaatttacatcacccccacaataa

SEQ ID NO: 48 - DNA, 327 bp, Coffea canephora
  source 1..327: mol_type = genomic DNA
ggagtccggactgctgctgctgctatcatcagtcgtgtttctcctgtcaaaagaaatttacatcacccccacaataatcttcaccctcttgtttttcctttgaatcaatacaaactagtgaaaaaggttggccttttttttttttttttctttaaagttcccttgagagcaaaaaagagagaatggaacatcagtttgtcaaaaagccagtatcttgaaggtaactcaagatgccttgttctagtatttctcccgactctctctctatatatccccctcacccttttcttcagttttttttcctttggattgattgctggggactga

SEQ ID NO: 49 - DNA, 78 bp, Coffea canephora
  source 1..78: mol_type = genomic DNA
tcttcaccctcttgtttttcctttgaatcaatacaaactagtgaaaaaggttggccttttttttttttttttctttaa

SEQ ID NO: 50 - DNA, 78 bp, Coffea canephora
  source 1..78: mol_type = genomic DNA
gagcaaaaaagagagaatggaacatcagtttgtcaaaaagccagtatcttgaaggtaactcaagatgccttgttctag

SEQ ID NO: 51 - DNA, 111 bp, Coffea canephora
  source 1..111: mol_type = genomic DNA
ctcaagatgccttgttctagtatttctcccgactctctctctatatatccccctcacccttttcttcagttttttttcctttggattgattgctggggactgaagggttga

SEQ ID NO: 52 - DNA, 210 bp, Coffea canephora
  source 1..210: mol_type = genomic DNA
ttgctggggactgaagggttgaatccatgtattttgcctatttttatatgggttgtttcaacaacattaaaaatgtatcttcgtcttcgcaattgcagtttattttcttccagtattttactctcgtcctgcaatccctttgtgtttaaagtacttcagatcctaatacttgtaatcttgttgtcttgtttcagagatttgattgattga

SEQ ID NO: 53 - DNA, 135 bp, Coffea canephora
  source 1..135: mol_type = genomic DNA
agggttgaatccatgtattttgcctatttttatatgggttgtttcaacaacattaaaaatgtatcttcgtcttcgcaattgcagtttattttcttccagtattttactctcgtcctgcaatccctttgtgtttaa

SEQ ID NO: 54 - DNA, 96 bp, Coffea canephora
  source 1..96: mol_type = genomic DNA
aaatgtatcttcgtcttcgcaattgcagtttattttcttccagtattttactctcgtcctgcaatccctttgtgtttaaagtacttcagatcctaa

SEQ ID NO: 55 - protein, 728 aa, Actinidia chinensis
MCGLIDLNTVLDGDETTAASFDSPTPSPPPSTSSGALALGSSSSSSSVCLELWHACAGPLTSLPKKGSVVVYFPQGHLEHASDFPAAAYDLPPHVFCRVLDVKLHAEAATDEVYAQVSLIPDTQIEQKWRQGEIEAESEEEDTEGAAKSTTPHMFCKTLTASDTSTHGGFSVPRRAAEDCFPPLDYKQQRPSQELFAKDLHGIEWRFRHIYRGQPRRHLLTTGWSAFVNKKKLVSGDAVLFLRGGNGELRLGIRRAAQVKNDSTFPALSCQKLNVGNINVVVNAISMRSVFNIWYNPRASSSEFIIPFCKFSKSLTHSFTVGMRFKMRFETEDSAERRYTGLITGVSDVDPARWPGSKWRCLLVRWDDLETIRHNRVSPWEIEPAGSVAASSSLMAPGMKRTRVGLPSTKPDFPIPRDGTGVSDFGESFRFHKVLQGQEITGFIYPYDGTDVHNHHPSEIRRCPPCSGSSKMSPIGNGVRNLLGDSGVSYENTGFGESLLFNKVLQGQETFSSSPYGRAPASNMAQGQGINDGLRASSSRNGWSHLLQGYNTYLRPSAPPIPVSSPSSVLMFHPARTPALNELRNQVLPDVSETYGGKPTSSSPCDRSFRKEDTQCVNFFGLLKEHNQLGVSHSTFATHSSYRGSQDLVSACKSSCRLFGFPLTEERNATNKECNPISSPYNPNASFRPHGEENLLSTPPLVTKIIGSSCTKVSDLCAVRDTLLDIPL

SEQ ID NO: 56 - DNA, 543 bp, Actinidia chinensis
  source 1..543: mol_type = genomic DNA
gcagcgctgctgcttctgcagctgctgcaacttgcaggtgagagaaatcacaaatctcacccctcacctcactacatgtagttccaagcatacacacatttatatacacatacgtataaatatatatatctgtataatcacacaaaagcatgatttgatatctaaaagtcccctgaaaatctagagagagaaattatagagcgagcgagtgagaattttatttctactcgtctcctttcacagtgtgaatcgcagggcgtctctctctctctctctctgtctgtctatatctacatattcgtatgtagttcgtatttccgccatttctgttaaatttctcaaagcttgatttgatgttgagaatctagagagagagtgagaatcttgtttcgactcatctgctttctccgactgcgaatcgcagggtttatctctctatttctctatataactccatatgtatcagtatcgcagttagtaattcgtccatttccgattgaatttcgccgttgatttgtgttctaggtttttttttgtgtgacg

SEQ ID NO: 57 - DNA, 81 bp, Actinidia chinensis
  source 1..81: mol_type = genomic DNA
gcagcgctgctgcttctgcagctgctgcaacttgcaggtgagagaaatcacaaatctcacccctcacctcactacatgtag

SEQ ID NO: 58 - DNA, 183 bp, Actinidia chinensis
  source 1..183: mol_type = genomic DNA
cagcgctgctgcttctgcagctgctgcaacttgcaggtgagagaaatcacaaatctcacccctcacctcactacatgtagttccaagcatacacacatttatatacacatacgtataaatatatatatctgtataatcacacaaaagcatgatttgatatctaaaagtcccctgaaaatctag

SEQ ID NO: 59 - DNA, 78 bp, Actinidia chinensis
  source 1..78: mol_type = genomic DNA
gagaaatcacaaatctcacccctcacctcactacatgtagttccaagcatacacacatttatatacacatacgtataa

SEQ ID NO: 60 - DNA, 168 bp, Actinidia chinensis
  source 1..168: mol_type = genomic DNA
aagtcccctgaaaatctagagagagaaattatagagcgagcgagtgagaattttatttctactcgtctcctttcacagtgtgaatcgcagggcgtctctctctctctctctctgtctgtctatatctacatattcgtatgtagttcgtatttccgccatttctgttaa

SEQ ID NO: 61 - DNA, 150 bp, Actinidia chinensis
  source 1..150: mol_type = genomic DNA
agcgagcgagtgagaattttatttctactcgtctcctttcacagtgtgaatcgcagggcgtctctctctctctctctctgtctgtctatatctacatattcgtatgtagttcgtatttccgccatttctgttaaatttctcaaagcttga

SEQ ID NO: 62 - DNA, 151 bp, Actinidia chinensis
  source 1..151: mol_type = genomic DNA
gaatctagagagagagtgagaatcttgtttcgactcatctgctttctccgactgcgaatcagggtttatctctctatttctctatataactccatatgtatcagtatcgcagttagtaattcgtccatttccgattgaatttcgccgttga

SEQ ID NO: 63 - DNA, 132 bp, Actinidia chinensis
  source 1..132: mol_type = genomic DNA
agagagagtgagaatcttgtttcgactcatctgctttctccgactgcgaatcgcagggtttatctctctatttctctatataactccatatgtatcagtatcgcagttagtaattcgtccatttccgattga

SEQ ID NO: 64 - protein, 799 aa, Malus domestica
MEIDLNHAVREVEKNAYCNGDCGGVCVYCLSSSTSSSSSNSSSAPVASSIYLELWHACAGPLISLPKKGNVVVYFPQGHLEQVASSSPFSPMEMPTFDLHPQIFCKVVNVQLLANKENDEVYTHVTLLPQPELVGTNLEGKELEELGVDEGDGGSPTKSTPHMFCKTLTASDTSTHGGFSVPRRAAEDCFPPLDYKQQRPSQELVAKDLHGVEWRFRHIYRGQPRRHLLTTGWSIFISQKNLVAGDAVLFLRGENGELRLGIRRAVRPRNGLPDSVVGNQNSYPSVLSFVANAISTKSMFHVFYSPRASHAEFVIPYQKYVKSITNPVTTGTRFKMRFDRDDSPERRCSGVVTGITDLDPFRWPNSKWRCLMVRWDEDIGNDHQERVSLWEVDPSVSLPPLSIQSSPRLKKLRTSLQGTPPIPSITAGGAGFKDFEESVKSSKVLQGQENIGFISPLYGGDTVNRPQDFEMHAPAHLSLASNATQKATIDELMRARRTPYTGFAESDRFPKVLQGQEICPLRSLTGKANFTLGDWESNNGCAPFNVYQPPKPNYFSLASGSLPNMYFPYGDIHRAGQDPTMCSNATNLPREKMQINPYSMQMGVARNEVGRPNKPCEHKTQESTSTLPALVPNPRNPNDKDYNGTVTGCKLFGFSLTGDNPSPNSQSSSKRSCTKVHKQGSLVGRAIDLAKLNGYGDLLTELERLFGMEGLLRDSDKGWRILYTDSENDMMVVGDDPWHEFCNVVSKIHIYTREEVEKMTIGMISDDTQSCLEHAPVMLEVSKSSSVSQPDSSPTAIRV

SEQ ID NO: 65 - DNA, 614 bp, Malus domestica
  source 1..614: mol_type = genomic DNA
gagagagagagactgtgttgggtgggggggacaggctaagacagtacattaccaacactctccaccctcctctctctctttctctcctcccctctctctctgtcttctgttaaacagtcgaagtcgaaaagccatcaccacattggctttttttgccttttgtttctgtgttcgtatgcaattgtctacgtctgctgctgcttctgctccttcttctaaccacctgtaaatgggggaggaggaaatggaatcatactttgaattccttgttatcatttgataaaccacttcttcttcttgctcttctctctctctcactctcagcttttctctctccttcactctctgaaattttcctccaagccaaacatgattttcttcttcttttctgttcatctctttttgctttctgggttcccttcaaaacatcaaaactccttgaaatgatttgaatttcacctccccatttcaaattttccctttctgctttctgggttctcctcacttctcccatttggttactcattttcttgtttttgattatcttgtcaatttttcttttgggactgcttatctgggaagcactttttcttgagcaattctggggaacattc

SEQ ID NO: 66 - DNA, 228 bp, Malus domestica
  source 1..228: mol_type = genomic DNA
agagagagagactgtgttgggtgggggggacaggctaagacagtacattaccaacactctccaccctcctctctctctttctctcctcccctctctctctgtcttctgttaaacagtcgaagtcgaaaagccatcaccacattggctttttttgccttttgtttctgtgttcgtatgcaattgtctacgtctgctgctgcttctgctccttcttctaaccacctgtaa

SEQ ID NO: 67 - DNA, 111 bp, Malus domestica
  source 1..111: mol_type = genomic DNA
gagagagagactgtgttgggtgggggggacaggctaagacagtacattaccaacactctccaccctcctctctctctttctctcctcccctctctctctgtcttctgttaa

SEQ ID NO: 68 - DNA, 180 bp, Malus domestica
  source 1..180: mol_type = genomic DNA
gacagtacattaccaacactctccaccctcctctctctctttctctcctcccctctctctctgtcttctgttaaacagtcgaagtcgaaaagccatcaccacattggctttttttgccttttgtttctgtgttcgtatgcaattgtctacgtctgctgctgcttctgctccttcttctaa

SEQ ID NO: 69 - DNA, 339 bp, Malus domestica
  source 1..339: mol_type = genomic DNA
acagtcgaagtcgaaaagccatcaccacattggctttttttgccttttgtttctgtgttcgtatgcaattgtctacgtctgctgctgcttctgctccttcttctaaccacctgtaaatgggggaggaggaaatggaatcatactttgaattccttgttatcatttgataaaccacttcttcttcttgctcttctctctctctcactctcagcttttctctctccttcactctctgaaattttcctccaagccaaacatgattttcttcttcttttctgttcatctctttttgctttctgggttcccttcaaaacatcaaaactccttgaaatgatttga

SEQ ID NO: 70 - DNA, 186 bp, Malus domestica
  source 1..186: mol_type = genomic DNA
attccttgttatcatttgataaaccacttcttcttcttgctcttctctctctctcactctcagcttttctctctccttcactctctgaaattttcctccaagccaaacatgattttcttcttcttttctgttcatctctttttgctttctgggttcccttcaaaacatcaaaactccttgaaatga

SEQ ID NO: 71 - DNA, 153 bp, Malus domestica
  source 1..153: mol_type = genomic DNA
aatgatttgaatttcacctccccatttcaaattttccctttctgctttctgggttctcctcacttctcccatttggttactcattttcttgtttttgattatcttgtcaatttttcttttgggactgcttatctgggaagcactttttcttga

SEQ ID NO: 72 - DNA, 93 bp, Malus domestica
  source 1..93: mol_type = genomic DNA
tttgaatttcacctccccatttcaaattttccctttctgctttctgggttctcctcacttctcccatttggttactcattttcttgtttttga

SEQ ID NO: 73 - DNA, 162 bp, Malus domestica
  source 1..162: mol_type = genomic DNA
atttcacctccccatttcaaattttccctttctgctttctgggttctcctcacttctcccatttggttactcattttcttgtttttgattatcttgtcaatttttcttttgggactgcttatctgggaagcactttttcttgagcaattctggggaacattc

SEQ ID NO: 74 - protein, 799 aa, Corylus avellana
MEIDLNHAVTEVEKNAYCNGDCGKGACVCCLSSSTSSCSSNSSGAPVSSSIYLELWHACAGPLTSLPKKGNVVVYFPQGHLEQAASSSPFSPSEMPSFDLHPQIFCRVVNVQLLANKENDEVYTQVTLLPQPELAGKNLDGKELEELGVDEEGGGGSPTKSTPHMFCKTLTASDTSTHGGFSVPRRAAEDCFPPLDYKQQRPSQELVAKDLHGVEWRFRHIYRGQPRRHLLTTGWSIFVSQKNLVAGDAVLFLRGENGELRLGIRRAVRPRNGLPDSIVGNENSYPKAFSLVANAISAKSMFHVFYSPRASHAEFVISYQKYVKSITNPVTIGTRFKMRFEMDDSPERRCSGVVTGISDLDPYRWPNSKWRCLMVRWDDEDIASDPQDRVSPWEIDPSVSLPPLSIQSSPRLKKLRTSLQATPPDNPVTAGGGGYLDFEESVRSPKVLQGQENVGFISPFYGCDTVNRLLDFEMRSVAHQNRASTGVGKSNNNEFLKTHPTTYTGFAESDRFPKVLQGQEICPLRSLTRKSEFNAWGKLNPGCNSFNVYQEPKPNFYPLQSESLKSLYFPYNDMHQAGKNPRMLSYASNLPRENVSFNSSSVQTGVITNEFGMRSLPNEHTPAENVSASPNLGANMRNTKDEKFNGNVTGCKLFGFSLTGETPNSHVSGKRSCTKVHKQGSLVGRAIDLSRLNGYGDLLTELERLFSMEGLLQDPDKGWRILYTDSENDVMVVGDDPWHEFCNVVSKIHIYTREEVEKMTIGMISDDTQSCLEQAQVVMEASKSSSVGQPDSSPTVIRV

SEQ ID NO: 75 - DNA, 502 bp, Corylus avellana
  source 1..502: mol_type = genomic DNA
aagtccaaaagccactaccgcattggcttttatcgtcttttgttctgtgcttcctatgcaattgtctatgtctgctgcttctgctccctcttctcagtacctgcaaatgggggaggaatcatactttgaatttcttgttattatttgatacccatctctttttcttctcatcttttagccctctggctgggtatccctccaaacatgactttcttgttgttttctataattctattcatccgcttccattcctctcctgtttctgctttgtgggtacccctcaaaaatatgaattccttaccatgatttgaccccatttccaattttatatagtttttcctttgtggtactcttcttatgaccacttgattactctaatcccctccttttttttttttcttttttgttttttgtttttttcttttcaatattttgttggtgggtgggttggttatttggttgtacttctattcatccacacaagaaccgcactggggtgttc

SEQ ID NO: 76 - DNA, 129 bp, Corylus avellana
  source 1..129: mol_type = genomic DNA
aagtccaaaagccactaccgcattggcttttatcgtcttttgttctgtgcttcctatgcaattgtctatgtctgctgcttctgctccctcttctcagtacctgcaaatgggggaggaatcatactttga

SEQ ID NO: 77 - DNA, 147 bp, Corylus avellana
  source 1..147: mol_type = genomic DNA
agtccaaaagccactaccgcattggcttttatcgtcttttgttctgtgcttcctatgcaattgtctatgtctgctgcttctgctccctcttctcagtacctgcaaatgggggaggaatcatactttgaatttcttgttattatttga

SEQ ID NO: 78 - DNA, 309 bp, Corylus avellana
  source 1..309: mol_type = genomic DNA
gtccaaaagccactaccgcattggcttttatcgtcttttgttctgtgcttcctatgcaattgtctatgtctgctgcttctgctccctcttctcagtacctgcaaatgggggaggaatcatactttgaatttcttgttattatttgatacccatctctttttcttctcatcttttagccctctggctgggtatccctccaaacatgactttcttgttgttttctataattctattcatccgcttccattcctctcctgtttctgctttgtgggtacccctcaaaaatatgaattccttaccatgatttga

SEQ ID NO: 79 - DNA, 177 bp, Corylus avellana
  source 1..177: mol_type = genomic DNA
atttcttgttattatttgatacccatctctttttcttctcatcttttagccctctggctgggtatccctccaaacatgactttcttgttgttttctataattctattcatccgcttccattcctctcctgtttctgctttgtgggtacccctcaaaaatatgaattccttaccatga

SEQ ID NO: 80 - DNA, 189 bp, Corylus avellana
  source 1..189: mol_type = genomic DNA
ccccatttccaattttatatagtttttcctttgtggtactcttcttatgaccacttgattactctaatcccctccttttttttttttcttttttgttttttgtttttttcttttcaatattttgttggtgggtgggttggttatttggttgtacttctattcatccacacaagaaccgcactggggtgt

SEQ ID NO: 81 - DNA, 140 bp, Corylus avellana
  source 1..140: mol_type = genomic DNA
ccacttgattactctaatcccctccttttttttttttcttttttgttttttgtttttttcttttcaatattttgttggtgggtgggttggttatttggttgtacttctattcatccacacaagaaccgcactggggtgtt

SEQ ID NO: 82 - DNA, 123 bp, Corylus avellana
  source 1..123: mol_type = genomic DNA
tcccctccttttttttttttcttttttgttttttgtttttttcttttcaatattttgttggtgggtgggttggttatttggttgtacttctattcatccacacaagaaccgcactggggtgtt

SEQ ID NO: 83 - DNA, 587 bp, Persea americana
  source 1..587: mol_type = genomic DNA
acttctcactctccattgattctttagacgaacgtcccaaagcagtctccattgattctctagatgagaaccccagattgatcaatgcagaaaaaaaatatactgcaacagaaatcaaagaaccctcaaaagcattaaaccccaattcctttgttcctatcttaaacagtacacacttctcagatcatgctgaaatcaatcaaagaaaacaaaaaaaacaaaaacccaacttcaggctagagagaaaaagatcaccaaacccatcaataaaaacttctcaccttttttctctctggattacccaaagaaccctcaaaagcattaaaccccaaaattcctctgttcctatcctaatcagtacatacttctcatataatgctgaaatcaatcaaactgaacagaaaacataaaaacccaacagcaggctggggagaaacagaacaccaaaccatcaataaaaatctcaccttcttctctctagatcattcctgcctgaaaaaaacaatcactgggttttcaagacactccacgagaaagcaagagaaggaagatgaagagagcaaagagcagagatgggtctctctctc

SEQ ID NO: 84 - DNA, 81 bp, Persea americana
  source 1..81: mol_type = genomic DNA
tcaatctggggttctcatctagagaatcaatggagactgctttgggacgttcgtctaaagaatcaatggagagtgagaagt

SEQ ID NO: 85 - DNA, 183 bp, Persea americana
  source 1..183: mol_type = genomic DNA
tctgagaagtgtgtactgtttaagataggaacaaaggaattggggtttaatgcttttgagggttctttgatttctgttgcagtatattttttttctgcattgatcaatctggggttctcatctagagaatcaatggagactgctttgggacgttcgtctaaagaatcaatggagagtgagaag

SEQ ID NO: 86 - DNA, 78 bp, Persea americana
  source 1..78: mol_type = genomic DNA
gaagtttttattgatgggtttggtgatctttttctctctagcctgaagttgggtttttgttttttttgttttctttga

SEQ ID NO: 87 - DNA, 87 bp, Persea americana
  source 1..87: mol_type = genomic DNA
gataggaacagaggaattttggggtttaatgcttttgagggttctttgggtaatccagagagaaaaaaggtgagaagtttttattga

SEQ ID NO: 88 - DNA, 111 bp, Persea americana
  source 1..111: mol_type = genomic DNA
ttgatttcagcattatatgagaagtatgtactgattaggataggaacagaggaattttggggtttaatgcttttgagggttctttgggtaatccagagagaaaaaaggtga

SEQ ID NO: 89 - DNA, 114 bp, Persea americana
  source 1..114: mol_type = genomic DNA
tctagagagaagaaggtgagatttttattgatggtttggtgttctgtttctccccagcctgctgttgggtttttatgttttctgttcagtttgattgatttcagcattatatga

SEQ ID NO: 90 - DNA, 93 bp, Persea americana
  source 1..93: mol_type = genomic DNA
agagaagaaggtgagatttttattgatggtttggtgttctgtttctccccagcctgctgttgggtttttatgttttctgttcagtttgattga

SEQ ID NO: 91 - DNA, 105 bp, Persea americana
  source 1..105: mol_type = genomic DNA
gagagagagacccatctctgctctttgctctcttcatcttccttctcttgctttctcgtggagtgtcttgaaaacccagtgattgtttttttcaggcaggaatga

SEQ ID NO: 92 - DNA, 81 bp, Persea americana
  source 1..81: mol_type = genomic DNA
agagagagacccatctctgctctttgctctcttcatcttccttctcttgctttctcgtggagtgtcttgaaaacccagtga

SEQ ID NO: 93 - protein, 695 aa, Persea americana
MIMFGDSSKELMKETEKCLDSQLWHACAGGMVQMPPVNSKVYYFPQGQAEHANGNVDFGNCPRIPALILCRVAAVKFLADPETDEVYAKIQLVPVRSGEADYLEDDGGLSGNGAEVADKPASFAKTLTQSDANNGGGFSVPRYCAETIFPRLDYSADPPVQTILAKDVHGEIWKFRHIYRGTPRRHLLTTGWSTFVNQKKLVAGDSIVFLRTENGDLCVGIRRAKRGVGGGPESPSGWNSSAGNCVSPFGGFSVFLKEDERLMRNANVGMSNSSGGGLRGRGRVKPDSVIEAAALAANGQPFEVVYYPRASTPEFCVKAAAVRAAMRIQWCSGMRFKMAFETEDSSRISWFMGTISSVQVADPARWPNSPWRLLQVTWDEPDLLQNVKRVSPWLVELVSNMPTIQLSPFSPPRKKLRLLQHPEFPLDNQFPTPTFSGNPLGYSSPLCCLSDNTPAGIQGARHAQFGISLSDLHFNKLQTGLFPPTSFQRLEHAAPPSRISSGVIISTPNIRDNVSCLLTIGNSSQGAKKLDDGKTPQLMLFGKPILTEQQISLSCSGDTVSPVRTGNSSSDGNQDKVTNFSSDGLPEHPSCEGFPWYKDRLTSELGLETGHCKVFMESEDVGRTLDLSVLGSYEELYRRLTNMFGIERSEMLSHVLYRDAAGAVKHTGDEPFSEFMKTARRLMILTDSGSDNIGR

SEQ ID NO: 94 - DNA, 6311 bp, Persea americana
  source 1..6311: mol_type = genomic DNA
caacttacaaacgtcaaaacaggaaacaaccaaaacccgctttaggacagcactagcaagtcctttgagaatgcttggagcatttcaggaatatttgtgtgtcctctcactttatcttctcctcctcctccttctcataatatcttcatctcctcttctcttctccttctcttcttctcttttcttctccaaactcaatcagcttcttctattctagagaaaacagagttaccagatctatatacaacacctctctgctctctctgcttgttctctttcatgtaatcttctatgcaaaaaaccatggaagaaaagacgagtctttgctgaaatccgagattctcgttatagaattcattctgggttcttaacgaaacttccaattgaggcttttctgcgccatatttgatgtctcttgggcggttttgatgaagaatctgaaatgggtcgaagcgatctctgcagttttttgaccgtttttgaagttcttcagctggagactgtactgcttctcttgttgaattagggactcttctatcggtggtcgggattgaagaagcgcttctaaaaaaaagccgattcgattttgcagaagtttatgaaatctttcgtcttgtatatctgtgttggagatactgcgagaaaatttggattttgaaaaagaggtgattttaggctttgttttgtaagagttgtttgggaaaatgggagatggaagatatgaaaaacgaggagtgttaaaagttgaaagaggatgtggaaaatgtgtaaaatgagccctttttgttatttttcttgattggtgaagaggatcatagctgaagttggtggaaagcatcatacaagatatggttgttttgggcatttttgggctttcttgaagcaaggttgtagaaaacgctaagaaatgtggtgcttctcttgaagatggaaatatttcacataggaggaaggaaagactagtttttgagaggaaaggtttgatgatttctttctctttctttcttttctctcttccttcctttctcttcttttttttcccctgaagctggttatctttgtgtggttgtagcattttctaagagaatacagctatattggaaatgggtttttactttgaagaaaatggagacagagaaatgaagctgctcagaggaaggttagagatttttactgttctgggttcctctctatcaagttggattacatccacaaaatcttttctgctttagctgtttggatttctctcttctgttctctcatctgggttttattcatatatgatttttcaggttttgttaatatttaattttcagcgtttgagaactacttactaggaagaaaggaattgggacttttttgctttgtgggtttctgttgttgtgttgttttgagatgatcatgtttggagattcatcaaaagaattaatgaaggagactgagaaatgcttggattctcagctctggcatgcatgtgctgggggaatggttcagatgccccctgtgaactccaaggtgtactacttcccacagggccaggctgagcatgccaatgggaatgttgattttgggaattgtcccaggattccagcgctgatcctctgcagagttgccgccgtgaaattcttggctgatccggagaccgacgaggtctatgcaaagattcaattagttccagtgaggagcggggaagctgattatttagaggatgatgggggtcttagtggcaatggtgcagaggttgcagacaagcctgcatcctttgcaaagacattgacacaatctgatgccaacaatggtgggggcttctctgtccctcgctactgtgcagagacgatcttcccccgcttagactattcagctgatcctcctgtccagactatactggccaaggatgttcatggtgagatttggaaattccggcatatttacagagggactccacgccggcatttgcttacaacggggtggagcactttcgtgaaccagaaaaagcttgttgctggtgattcaatagtgttcttgagaacggaaaatggggatctctgtgttggaatccggcgagcgaagagaggggtcggcggtgggcctgagtctccgtctggatggaattcatctgccgggaattgcgtctcaccattcggtggattctccgttttcttgaaggaagatgaaaggctgatgagaaatgctaatgtggggatgtcgaattcctctggaggaggtttgaggggaaggggcagagtgaaaccagactctgtcattgaggccgcggctcttgctgcaaatggtcagccttttgaggttgtctactatccgcgcgcaagcactccagagttttgtgtaaaggccgcagctgtcagagcagcaatgaggatccaatggtgttcaggaatgaggtttaagatggcctttgagactgaggattcttcacggattagctggtttatgggtaccatatcctctgtccaggttgctgatcctgctcgatggcctaattcgccatggaggcttcttcaggtacccatttctccatcccactgtttctcttagtctcttacaaaatatgttgttatctgtatgccattgcggacagggtgactattttatttgctagctaaccacagatttttatggtaattttgaaattttcgttcctgtttttgttaccaaaaaatgaaaaacaaatttgagtagttggaatgtgttaaccccaaaggcctcagattaaccaaagaattttaggttactcattggaggggaaaaaaggagaataattgggatgtgttaacacaaaaggcctcagattaaccaaagagtgtctagttactcattggcactactgtatttgtcttcacactaatcagcctcatattggctctcaagtttcttgtcaatggtttaattttgtagtcagctatatggtctccatgaagtttttattttcattcaactctgtcggcatctcaagttacatgacattactaatttctagttaataaggatgatatctgcatactctcttgtggatcttctgttacacctatgggtctttagtgatgttctaaaatccaaaccggaccagttggactggattcaacagaaactgctgcctggaggttacattccaaactaaatcattctatgagcaactgaccactccagtactgctaaaaaatgttgaatctaatactaaccgttgttcagatcaggagcaatgaatgtttttgagtagttgagcatttattattattattattacttgacctttttctgtcatttgatgccactggtaagctgggtcgaaccggtttggtctcaatcatcaacttcagttgttcaaccttcagatcgttacatgcagggctgcttccctctggaatcttatgatcctttagcgttttggtcattttttgtttcccaactttcataactatagcaataaattaagaacctgcaatttgcaagtgaaaagggccttcacatggtattggcatactgcattgtttaaggtggcgtaggtattcaaaggtcagatgtgttctgatgtttgtctgtcttgaatgatctatggttacatgaacattcaaatgagtatggccctggcccataactataccattattacgaaaaaggaacagaaaatagaaacttaaccagagtgatgttatgtgaactttacagccaacaccagatttggatgggataaggctttattaatcatgaggatgatgtaatgtgaattttataattatatgcactttcattttccatttattgttgtcatgtatggagtgcataaataacattgcattgatttaggtatagattacgatcttgtaaatgaatcataaatacccccaccagcaaaaaggaaaaaaaggaagagagagagagagaccatcaatgttgtattaccataggttatctcgaacaattgagtgcatgcaaagcccctgtgatctccgtcttataaaaatttataaaattgtctgagaatcttgctggtaaattttagcaagcattctttcttttccttttcttttctatttcttttttttttttgggattgctacagtgaagaaaagagtgatcctcattcaaataggctatggttgcctgtgagaaatcttgggcaaatattcggtttcatggtccactgcagtttttttgttgttcattagcacattagattacagtactaagcagtccatttatctgctattcataacttgagatcttatacattttttttagcatttccagctcttatcttttgaatgcaaaatgcatatttcaggtgacatgggatgaaccagatttactgcagaatgttaaacgcgtcagcccatggttggttgaattggtgtcaaacatgccaaccatccaactttcacccttctcaccacctaggaaaaagttgcggcttcttcagcaccctgagtttcctcttgacaatcagtttccaacacccactttttccggcaaccccctcgggtacagcagcccattgtgctgtctatcagacaacactcctgcaggcatacagggagccaggcatgctcaatttggtatatctttatcagatctccactttaacaaactgcagacgggtctgtttccacccaccagttttcagcggcttgaacatgctgccccaccctccagaatctcctccggcgttatcatcagcaccccaaacattcgtgataatgtctcttgtttactgactataggaaattcttcacaaggtgcaaagaaacttgatgatggaaagacaccccagctaatgctctttgggaaaccaatactcactgagcagcaaatatctttaagctgttctggtgacacggtctcccctgtgcgtactggaaacagttcttctgatggaaatcaagataaggtgacaaatttctcatctgatgggctgccagaacacccatcgtgtgaaggatttccatggtataaggatcgtctaacaagtgagcttggtttggaaactgggcactgtaaggtgttcatggagtcggaggatgtaggtcgaactcttgatctgtcagttcttggctcatacgaagaactatacagaaggctgaccaacatgtttggcattgaaagatctgagatgctgagccacgtgctttaccgtgatgcagctggtgcagttaagcacactggagacgaaccatttaggtaaagatttttctttaggttgttactatatatatgtactgaaatattcttatcaagctttagaaatgtcctaatataccaaaaaatcagttacacacatacttcattttcatgcactatacagaattagatcccttcaactcaaccccagattgatgggataagctaagttgctggtggccaaaatctgaactagttaaaggggcagttgatgacttgtgcctactagaaacatctttgctttccaatttctttcccttcttaccattcaaaaaaataaaaaatcttacccttctttctcgatcgtttttcaagtgtattgacattttgaactatgtattaagtgcctttctcttgttgcagcgagttcatgaaaacagcgcgaagactgatgattctgacggattcaggcagcgacaacataggaaggtagaaagtactccttcatttgttaaatgtacagttgaatgggcaaattcttctgttcaagaccaactttatattgttgttcaaggaactgttttatccttttttttttccgagtatcttgtaagtcaactgcaaagattctaaacggtcagatcccgatatttatcaagttcattctgcaaaatgcatgattacagcctcttttttttcaggcaaattctaccagtatgcattggtatctgatgcaatgccccgattttattgcacatagccatgtgtaagcttgtgggtaagcactggaagtagggtatggttactgtccattatctgcattggtatgtatatcattttatgtattcttgggagaactgaaagaccttttggacatgatgatcagatcaatgcataggctatgtatcagctgaaatccaatcagctttaggatgcacccaggaaaatatgtgcaagttacatttcctgtttattgcaaagtagtttggttaatgagctcttatcctctttcactgtaattttgttattttgaagtttctgctctaattgagcttcagtttgtttccaagtgtttatggacttcgatataactggtgatggtgttggttgactctcatagttctttgcgaattattttctccggttgtagggatatttttaaatttcggacttttctc

SEQ ID NO: 95 - DNA, 216 bp, Persea americana
  source 1..216: mol_type = genomic DNA
aacttacaaacgtcaaaacaggaaacaaccaaaacccgctttaggacagcactagcaagtcctttgagaatgcttggagcatttcaggaatatttgtgtgtcctctcactttatcttctcctcctcctccttctcataatatcttcatctcctcttctcttctccttctcttcttctcttttcttctccaaactcaatcagcttcttctattctag

SEQ ID NO: 96 - DNA, 240 bp, Persea americana
  source 1..240: mol_type = genomic DNA
gacagcactagcaagtcctttgagaatgcttggagcatttcaggaatatttgtgtgtcctctcactttatcttctcctcctcctccttctcataatatcttcatctcctcttctcttctccttctcttcttctcttttcttctccaaactcaatcagcttcttctattctagagaaaacagagttaccagatctatatacaacacctctctgctctctctgcttgttctctttcatgtaa

SEQ ID NO: 97 - DNA, 231 bp, Persea americana
  source 1..231: mol_type = genomic DNA
tatcttcatctcctcttctcttctccttctcttcttctcttttcttctccaaactcaatcagcttcttctattctagagaaaacagagttaccagatctatatacaacacctctctgctctctctgcttgttctctttcatgtaatcttctatgcaaaaaaccatggaagaaaagacgagtctttgctgaaatccgagattctcgttatagaattcattctgggttcttaa

SEQ ID NO: 98 - DNA, 192 bp, Persea americana
  source 1..192: mol_type = genomic DNA
agaaaacagagttaccagatctatatacaacacctctctgctctctctgcttgttctctttcatgtaatcttctatgcaaaaaaccatggaagaaaagacgagtctttgctgaaatccgagattctcgttatagaattcattctgggttcttaacgaaacttccaattgaggcttttctgcgccatatttga

SEQ ID NO: 99 - DNA, 102 bp, Persea americana
  source 1..102: mol_type = genomic DNA
cgaaacttccaattgaggcttttctgcgccatatttgatgtctcttgggcggttttgatgaagaatctgaaatgggtcgaagcgatctctgcagttttttga

SEQ ID NO: 100 - DNA, 117 bp, Persea americana
  source 1..117: mol_type = genomic DNA
tgtctcttgggcggttttgatgaagaatctgaaatgggtcgaagcgatctctgcagttttttgaccgtttttgaagttcttcagctggagactgtactgcttctcttgttgaattag

SEQ ID NO: 101 - DNA, 186 bp, Persea americana
  source 1..186: mol_type = genomic DNA
agttcttcagctggagactgtactgcttctcttgttgaattagggactcttctatcggtggtcgggattgaagaagcgcttctaaaaaaaagccgattcgattttgcagaagtttatgaaatctttcgtcttgtatatctgtgttggagatactgcgagaaaatttggattttgaaaaagaggtga

SEQ ID NO: 102 - DNA, 90 bp, Persea americana
  source 1..90: mol_type = genomic DNA
aaaaaagccgattcgattttgcagaagtttatgaaatctttcgtcttgtatatctgtgttggagatactgcgagaaaatttggattttga

SEQ ID NO: 103 - DNA, 87 bp, Persea americana
  source 1..87: mol_type = genomic DNA
aatctttcgtcttgtatatctgtgttggagatactgcgagaaaatttggattttgaaaaagaggtgattttaggctttgttttgtaa

SEQ ID NO: 104 - DNA, 87 bp, Persea americana
  source 1..87: mol_type = genomic DNA
gagttgtttgggaaaatgggagatggaagatatgaaaaacgaggagtgttaaaagttgaaagaggatgtggaaaatgtgtaaaatga

SEQ ID NO: 105 - DNA, 105 bp, Persea americana
  source 1..105: mol_type = genomic DNA
ttggtgaagaggatcatagctgaagttggtggaaagcatcatacaagatatggttgttttgggcatttttgggctttcttgaagcaaggttgtagaaaacgctaa

SEQ ID NO: 106 - DNA, 165 bp, Persea americana
  source 1..165: mol_type = genomic DNA
agcaaggttgtagaaaacgctaagaaatgtggtgcttctcttgaagatggaaatatttcacataggaggaaggaaagactagtttttgagaggaaaggtttgatgatttctttctctttctttcttttctctcttccttcctttctcttcttttttttcccctga

SEQ ID NO: 107 - DNA, 102 bp, Persea americana
  source 1..102: mol_type = genomic DNA
gaggaaaggtttgatgatttctttctctttctttcttttctctcttccttcctttctcttcttttttttcccctgaagctggttatctttgtgtggttgtag

SEQ ID NO: 108 - DNA, 156 bp, Persea americana
  source 1..156: mol_type = genomic DNA
tttctttctctttctttcttttctctcttccttcctttctcttcttttttttcccctgaagctggttatctttgtgtggttgtagcattttctaagagaatacagctatattggaaatgggtttttactttgaagaaaatggagacagagaaatga

SEQ ID NO: 109 - DNA, 150 bp, Persea americana
  source 1..150: mol_type = genomic DNA
gagaatacagctatattggaaatgggtttttactttgaagaaaatggagacagagaaatgaagctgctcagaggaaggttagagatttttactgttctgggttcctctctatcaagttggattacatccacaaaatcttttctgctttag

SEQ ID NO: 110 - DNA, 189 bp, Persea americana
  source 1..189: mol_type = genomic DNA
agaaaatggagacagagaaatgaagctgctcagaggaaggttagagatttttactgttctgggttcctctctatcaagttggattacatccacaaaatcttttctgctttagctgtttggatttctctcttctgttctctcatctgggttttattcatatatgatttttcaggttttgttaatatttaa

SEQ ID NO: 111 - DNA, 120 bp, Persea americana
  source 1..120: mol_type = genomic DNA
agatttttactgttctgggttcctctctatcaagttggattacatccacaaaatcttttctgctttagctgtttggatttctctcttctgttctctcatctgggttttattcatatatga

SEQ ID NO: 112 - DNA, 105 bp, Persea americana
  source 1..105: mol_type = genomic DNA
ctgtttggatttctctcttctgttctctcatctgggttttattcatatatgatttttcaggttttgttaatatttaattttcagcgtttgagaactacttactag

SEQ ID NO: 113 - DNA, 84 bp, Persea americana
  source 1..84: mol_type = genomic DNA
ttttcagcgtttgagaactacttactaggaagaaaggaattgggacttttttgctttgtgggtttctgttgttgtgttgttttg

SEQ ID NO: 114 - protein, 1086 aa, Arabidopsis thaliana
MKAPSNGFLPSSNEGEKKPINSQLWHACAGPLVSLPPVGSLVVYFPQGHSEQVAASMQKQTDFIPNYPNLPSKLICLLHSVTLHADTETDEVYAQMTLQPVNKYDREALLASDMGLKLNRQPTEFFCKTLTASDTSTHGGFSVPRRAAEKIFPPLDFSMQPPAQEIVAKDLHDTTWTFRHIYRGQPKRHLLTTGWSVFVSTKRLFAGDSVLFVRDEKSQLMLGIRRANRQTPTLSSSVISSDSMHIGILAAAAHANANSSPFTIFFNPRASPSEFVVPLAKYNKALYAQVSLGMRFRMMFETEDCGVRRYMGTVTGISDLDPVRWKGSQWRNLQVGWDESTAGDRPSRVSIWEIEPVITPFYICPPPFFRPKYPRQPGMPDDELDMENAFKRAMPWMGEDFGMKDAQSSMFPGLSLVQWMSMQQNNPLSGSATPQLPSALSSFNLPNNFASNDPSKLLNFQSPNLSSANSQFNKPNTVNHISQQMQAQPAMVKSQQQQQQQQQQHQHQQQQLQQQQQLQMSQQQVQQQGIYNNGTIAVANQVSCQSPNQPTGFSQSQLQQQSMLPTGAKMTHQNINSMGNKGLSQMTSFAQEMQFQQQLEMHNSSQLLRNQQEQSSLHSLQQNLSQNPQQLQMQQQSSKPSPSQQLQLQLLQKLQQQQQQQSIPPVSSSLQPQLSALQQTQSHQLQQLLSSQNQQPLAHGNNSFPASTFMQPPQIQVSPQQQGQMSNKNLVAAGRSHSGHTDGEAPSCSTSPSANNTGHDNVSPTNFLSRNQQQGQAASVSASDSVFERASNPVQELYTKTESRISQGMMNMKSAGEHFRFKSAVTDQIDVSTAGTTYCPDVVGPVQQQQTFPLPSFGFDGDCQSHHPRNNLAFPGNLEAVTSDPLYSQKDFQNLVPNYGNTPRDIETELSSAAISSQSFGIPSIPFKPGCSNEVGGINDSGIMNGGGLWPNQTQRMRTYTKVQKRGSVGRSIDVTRYSGYDELRHDLARMFGIEGQLEDPLTSDWKLVYTDHENDILLVGDDPWEEFVNCVQNIKILSSVEVQQMSLDGDLAAIPTTNQACSETDSGNAWKVHYEDTSAAASFNR

SEQ ID NO: 115 - DNA, 60 bp, Arabidopsis thaliana
  source 1..60: mol_type = genomic DNA
tgagaatctgcagaaacacgagaaaaaaaagaaaagtttcaaagctttttaagagcttaa

SEQ ID NO: 116 - DNA, 63 bp, Arabidopsis thaliana
  source 1..63: mol_type = genomic DNA
tgaagcttcaaatattcttatgaactaaaaagaagaaaaaagcttttgtttctttttccttag

SEQ ID NO: 117 - DNA, 54 bp, Arabidopsis thaliana
  source 1..54: mol_type = genomic DNA
tgaactaaaaagaagaaaaaagcttttgtttctttttccttagcagcagaatga

SEQ ID NO: 118 - DNA, 78 bp, Arabidopsis thaliana
  source 1..78: mol_type = genomic DNA
taaaaagaagaaaaaagcttttgtttctttttccttagcagcagaatgatttttgtttccaaaattattactatttag

SEQ ID NO: 119 - DNA, 66 bp, Arabidopsis thaliana
  source 1..66: mol_type = genomic DNA
tagcagcagaatgatttttgtttccaaaattattactatttagtttctctcgtgctcttctcttga

SEQ ID NO: 120 - DNA, 147 bp, Arabidopsis thaliana
  source 1..147: mol_type = genomic DNA
tgatttttgtttccaaaattattactatttagtttctctcgtgctcttctcttgagcaaatacagattcgttaattttgctgaagaagaagaactctgtttcttccctgcaccaaaccaattttttcgttctttctataaaccatga

SEQ ID NO: 121 - DNA, 309 bp, Arabidopsis thaliana
  source 1..309: mol_type = genomic DNA
tgagcaaatacagattcgttaattttgctgaagaagaagaactctgtttcttccctgcaccaaaccaattttttcgttctttctataaaccatgaaagctccatcaaatggatttcttccaagttccaacgaaggagagaagaagccaatcaattctcaactatggcacgcttgtgcagggcctttagtttcattacctcctgtgggaagtcttgtggtttacttccctcaaggacacagcgagcaagttgcagcatcgatgcagaagcaaacagattttataccaaattacccaaatcttccttctaa

SEQ ID NO: 122 - DNA, 60 bp, Arabidopsis thaliana
  source 1..60: mol_type = genomic DNA
tgaagaagaagaactctgtttcttccctgcaccaaaccaattttttcgttctttctataa

SEQ ID NO: 123 - protein, 902 aa, Arabidopsis thaliana
MMASLSCVEDKMKTSCLVNGGGTITTTTSQSTLLEEMKLLKDQSGTRKPVINSELWHACAGPLVCLPQVGSLVYYFSQGHSEQVAVSTRRSATTQVPNYPNLPSQLMCQVHNVTLHADKDSDEIYAQMSLQPVHSERDVFPVPDFGMLRGSKHPTEFFCKTLTASDTSTHGGFSVPRRAAEKLFPPLDYSAQPPTQELVVRDLHENTWTFRHIYRGQPKRHLLTTGWSLFVGSKRLRAGDSVLFIRDEKSQLMVGVRRANRQQTALPSSVLSADSMHIGVLAAAAHATANRTPFLIFYNPRACPAEFVIPLAKYRKAICGSQLSVGMRFGMMFETEDSGKRRYMGTIVGISDLDPLRWPGSKWRNLQVEWDEPGCNDKPTRVSPWDIETPESLFIFPSLTSGLKRQLHPSYFAGETEWGSLIKRPLIRVPDSANGIMPYASFPSMASEQLMKMMMRPHNNQNVPSFMSEMQQNIVMGNGGLLGDMKMQQPLMMNQKSEMVQPQNKLTVNPSASNTSGQEQNLSQSMSAPAKPENSTLSGCSSGRVQHGLEQSMEQASQVTTSTVCNEEKVNQLLQKPGASSPVQADQCLDITHQIYQPQSDPINGFSFLETDELTSQVSSFQSLAGSYKQPFILSSQDSSAVVLPDSTNSPLFHDVWDTQLNGLKFDQFSPLMQQDLYASQNICMSNSTTSNILDPPLSNTVLDDFCAIKDTDFQNHPSGCLVGNNNTSFAQDVQSQITSASFADSQAFSRQDFPDNSGGTGTSSSNVDFDDCSLRQNSKGSSWQKIATPRVRTYTKVQKTGSVGRSIDVTSFKDYEELKSAIECMFGLEGLLTHPQSSGWKLVYVDYESDVLLVGDDPWEEFVGCVRCIRILSPTEVQQMSEEGMKLLNSAGINDLKTSVS

SEQ ID NO: 124 - DNA, 51 bp, Arabidopsis thaliana
  source 1..51: mol_type = genomic DNA
tgatggcacctttggtttctcttcttcattgaagggtcgaatttggtctga

SEQ ID NO: 125 - DNA, 93 bp, Arabidopsis thaliana
  source 1..93: mol_type = genomic DNA
tgaagggtcgaatttggtctgagaaaagaagagtggagagagagaaagagaagaggcaagagccccacaagagagacatgcttgtctgtgtag

SEQ ID NO: 126 - DNA, 201 bp, Arabidopsis thaliana
  source 1..201: mol_type = genomic DNA
tgagaaaagaagagtggagagagagaaagagaagaggcaagagccccacaagagagacatgcttgtctgtgtagtccctattttgtatgttctttaatctctcccactctgtttttgtcatgtgtaagggttttgtccttcgattttggagtcttctttaaccaaaaaaaaacagattccatgaaatggctttgtctttaa

SEQ ID NO: 127 - DNA, 90 bp, Arabidopsis thaliana
  source 1..90: mol_type = genomic DNA
tagtccctattttgtatgttctttaatctctcccactctgtttttgtcatgtgtaagggttttgtccttcgattttggagtcttctttaa

SEQ ID NO: 128 - DNA, 60 bp, Arabidopsis thaliana
  source 1..60: mol_type = genomic DNA
taagggttttgtccttcgattttggagtcttctttaaccaaaaaaaaacagattccatga

SEQ ID NO: 129 - DNA, 78 bp, Arabidopsis thaliana
  source 1..78: mol_type = genomic DNA
taaccaaaaaaaaacagattccatgaaatggctttgtctttaattcctctggtttgtcctggatttatcttcttgtag

SEQ ID NO: 130 - DNA, 102 bp, Arabidopsis thaliana
  source 1..102: mol_type = genomic DNA
tgaaatggctttgtctttaattcctctggtttgtcctggatttatcttcttgtagcctctttgctcaatagagctctttttgtctcttcttgtattaaataa

SEQ ID NO: 131 - DNA, 54 bp, Arabidopsis thaliana
  source 1..54: mol_type = genomic DNA
taattcctctggtttgtcctggatttatcttcttgtagcctctttgctcaatag

SEQ ID NO: 132 - DNA, 60 bp, Arabidopsis thaliana
  source 1..60: mol_type = genomic DNA
tagcctctttgctcaatagagctctttttgtctcttcttgtattaaataaaataaaatga

SEQ ID NO: 133 - DNA, 51 bp, Arabidopsis thaliana
  source 1..51: mol_type = genomic DNA
tgaagacttagtaaagaaaaagaaaaactcttgagtcttgctaaagcctag

SEQ ID NO: 134 - protein, 935 aa, Arabidopsis thaliana
MRLSSAGFNPQPHEVTGEKRVLNSELWHACAGPLVSLPPVGSRVVYFPQGHSEQVAASTNKEVDAHIPNYPSLHPQLICQLHNVTMHADVETDEVYAQMTLQPLNAQEQKDPYLPAELGVPSRQPTNYFCKTLTASDTSTHGGFSVPRRAAEKVFPPLDYSQQPPAQELMARDLHDNEWKFRHIFRGQPKRHLLTTGWSVFVSAKRLVAGDSVLFIWNDKNQLLLGIRRANRPQTVMPSSVLSSDSMHLGLLAAAAHAAATNSRFTIFYNPRASPSEFVIPLAKYVKAVYHTRVSVGMRFRMLFETEESSVRRYMGTITGICDLDPTRWANSHWRSVKVGWDESTAGERQPRVSLWEIEPLTTFPMYPSPFPLRLKRPWPPGLPSFHGLKEDDMGMSMSSPLMWDRGLQSLNFQGMGVNPWMQPRLDTSGLLGMQNDVYQAMAAAALQDMRGIDPAKAAASLLQFQNSPGFSMQSPSLVQPQMLQQQLSQQQQQLSQQQQQQQQLSQQQQQQLSQQQQQQLSQQQQQQLSQQQQQQAYLGVPETHQPQSQAQSQSNNHLSQQQQQVVDNHNPSASSAAVVSAMSQFGSASQPNTSPLQSMTSLCHQQSFSDTNGGNNPISPLHTLLSNFSQDESSQLLHLTRTNSAMTSSGWPSKRPAVDSSFQHSGAGNNNTQSVLEQLGQSHTSNVPPNAVSLPPFPGGRECSIEQEGSASDPHSHLLFGVNIDSSSLLMPNGMSNLRSIGIEGGDSTTLPFTSSNFNNDFSGNLAMTTPSSCIDESGFLQSSENLGSENPQSNTFVKVYKSGSFGRSLDISKFSSYHELRSELARMFGLEGQLEDPVRSGWQLVFVDRENDVLLLGDDPWPEFVSSVWCIKILSPQEVQQMGKRGLELLNSAPSSNNVDKLPSNGNCDDFGNRSDPRNLGNGIASVGGSFNY

SEQ ID NO: 135 - DNA, 75 bp, Arabidopsis thaliana
  source 1..75: mol_type = genomic DNA
tgattctcatttcaaataagagagagagagagagaagtaagtaaaactttagcagagagaagaataaacaaataa

SEQ ID NO: 136 - DNA, 99 bp, Arabidopsis thaliana
  source 1..99: mol_type = genomic DNA
taaaactttagcagagagaagaataaacaaataattatagcaccgtcacgtcgccgccgtatttcgttaccggaaaaaaaaaatcattcttcaacataa

SEQ ID NO: 137 - DNA, 207 bp, Arabidopsis thaliana
  source 1..207: mol_type = genomic DNA
taaacaaataattatagcaccgtcacgtcgccgccgtatttcgttaccggaaaaaaaaaatcattcttcaacataaaaataaaaacagtctctttctttctatctttgtctatctttgattattctctgtgtacccatgttctgcaacagttgagcaagtgcatgccccatatctctctgtttctcatttcccgatctttgcattaa

SEQ ID NO: 138 - DNA, 105 bp, Arabidopsis thaliana
  source 1..105: mol_type = genomic DNA
tagcaccgtcacgtcgccgccgtatttcgttaccggaaaaaaaaaatcattcttcaacataaaaataaaaacagtctctttctttctatctttgtctatctttga

SEQ ID NO: 139 - DNA, 75 bp, Arabidopsis thaliana
  source 1..75: mol_type = genomic DNA
taaaaacagtctctttctttctatctttgtctatctttgattattctctgtgtacccatgttctgcaacagttga

SEQ ID NO: 140 - DNA, 108 bp, Arabidopsis thaliana
  source 1..108: mol_type = genomic DNA
tgattattctctgtgtacccatgttctgcaacagttgagcaagtgcatgccccatatctctctgtttctcatttcccgatctttgcattaatcatatacttcgcctga

SEQ ID NO: 141 - DNA, 285 bp, Arabidopsis thaliana
  source 1..285: mol_type = genomic DNA
tgagcaagtgcatgccccatatctctctgtttctcatttcccgatctttgcattaatcatatacttcgcctgagatctcgattaagccagcttatagaagaagaaacggcaccagcttctgtcgttttagttagctcgagatctgtgtttctttttttcttggcttctgagcttttggcggtggtgggtttttctggagaaacccaaacgactatcaaagttttgttttttacaattttaagtgggagttatgagtggggtggattaagtaagttacaagtatga

SEQ ID NO: 142 - DNA, 81 bp, Arabidopsis thaliana
  source 1..81: mol_type = genomic DNA
taatcatatacttcgcctgagatctcgattaagccagcttatagaagaagaaacggcaccagcttctgtcgttttagttag

SEQ ID NO: 143 - DNA, 114 bp, Arabidopsis thaliana
  source 1..114: mol_type = genomic DNA
tagttagctcgagatctgtgtttctttttttcttggcttctgagcttttggcggtggtgggtttttctggagaaacccaaacgactatcaaagttttgttttttacaattttaa

SEQ ID NO: 144 - DNA, 87 bp, Arabidopsis thaliana
  source 1..87: mol_type = genomic DNA
tgagcttttggcggtggtgggtttttctggagaaacccaaacgactatcaaagttttgttttttacaattttaagtgggagttatga

SEQ ID NO: 145 - DNA, 51 bp, Arabidopsis thaliana
  source 1..51: mol_type = genomic DNA
taagtaagttacaagtatgaaggagttgaagattcgaagaagcggttttga

SEQ ID NO: 146 - DNA, 84 bp, Arabidopsis thaliana
  source 1..84: mol_type = genomic DNA
taagttacaagtatgaaggagttgaagattcgaagaagcggttttgaagtcggcgagaccaagattgcgagcttatttggctga

SEQ ID NO: 147 - DNA, 126 bp, Arabidopsis thaliana
  source 1..126: mol_type = genomic DNA
tgaagattcgaagaagcggttttgaagtcggcgagaccaagattgcgagcttatttggctgatgatttatttcagggaagaagaaataaatctgttttttttagggtttttagatttggttggtga

SEQ ID NO: 148 - DNA, 150 bp, Arabidopsis thaliana
  source 1..150: mol_type = genomic DNA
tgaagtcggcgagaccaagattgcgagcttatttggctgatgatttatttcagggaagaagaaataaatctgttttttttagggtttttagatttggttggtgaatgggtgggaggtggagggaaacagttaaaaaagttatgcttttag

SEQ ID NO: 149 - DNA, 84 bp, Arabidopsis thaliana
  source 1..84: mol_type = genomic DNA
tgaatgggtgggaggtggagggaaacagttaaaaaagttatgcttttagtgtctcttcttcataattacatttgggcatcttga

SEQ ID NO: 150 - DNA, 114 bp, Arabidopsis thaliana
  source 1..114: mol_type = genomic DNA
tagtgtctcttcttcataattacatttgggcatcttgaaatctttggatctttgaagaaacaaagttgtgtttttttttttgttctttgttgtttgctttttaagttagaataa

SEQ ID NO: 151 - DNA, 69 bp, Arabidopsis thaliana
  source 1..69: mol_type = genomic DNA
tgaaatctttggatctttgaagaaacaaagttgtgtttttttttttgttctttgttgtttgctttttaa

SEQ ID NO: 152 - DNA, 57 bp, Arabidopsis thaliana
  source 1..57: mol_type = genomic DNA
tgaagaaacaaagttgtgtttttttttttgttctttgttgtttgctttttaagttag

SEQ ID NO: 153 - DNA, 57 bp, Arabidopsis thaliana; identical to SEQ ID NO: 152
  source 1..57: mol_type = genomic DNA

SEQ ID NO: 154 - protein, 608 aa, Arabidopsis thaliana; identical to SEQ ID NO: 1

SEQ ID NO: 155 - protein, 1165 aa, Arabidopsis thaliana
MKAPSSNGVSPNPVEGERRNINSELWHACAGPLISLPPAGSLVVYFPQGHSEQVAASMQKQTDFIPSYPNLPSKLICMLHNVTLNADPETDEVYAQMTLQPVNKYDRDALLASDMGLKLNRQPNEFFCKTLTASDTSTHGGFSVPRRAAEKIFPALDFSMQPPCQELVAKDIHDNTWTFRHIYRGQPKRHLLTTGWSVFVSTKRLFAGDSVLFIRDGKAQLLLGIRRANRQQPALSSSVISSDSMHIGVLAAAAHANANNSPFTIFYNPRWAAPAEFVVPLAKYTKAMYAQVSLGMRFRMIFETEECGVRRYMGTVTGISDLDPVRWKNSQWRNLQIGWDESAAGDRPSRVSVWDIEPVLTPFYICPPPFFRPRFSGQPGMPDDETDMESALKRAMPWLDNSLEMKDPSSTIFPGLSLVQWMNMQQQNGQLPSAAAQPGFFPSMLSPTAALHNNLGGTDDPSKLLSFQTPHGGISSSNLQFNKQNQQAPMSQLPQPPTTLSQQQQLQQLLHSSLNHQQQQSQSQQQQQQQQLLQQQQQLQSQQHSNNNQSQSQQQQQLLQQQQQQQLQQQHQQPLQQQTQQQQLRTQPLQSHSHPQPQQLQQHKLQQLQVPQNQLYNGQQAAQQHQSQQASTHHLQPQLVSGSMASSVITPPSSSLNQSFQQQQQQSKQLQQAHHHLGASTSQSSVIETSKSSSNLMSAPPQETQFSRQVEQQQPPGLNGQNQQTLLQQKAHQAQAQQIFQQSLLEQPHIQFQLLQRLQQQQQQQFLSPQSQLPHHQLQSQQLQQLPTLSQGHQFPSSCTNNGLSTLQPPQMLVSRPQEKQNPPVGGGVKAYSGITDGGDAPSSSTSPSTNNCQISSSGFLNRSQSGPAILIPDAAIDMSGNLVQDLYSKSDMRLKQELVGQQKSKASLTDHQLEASASGTSYGLDGGENNRQQNFLAPTFGLDGDSRNSLLGGANVDNGFVPDTLLSRGYDSQKDLQNMLSNYGGVTNDIGTEMSTSAVRTQSFGVPNVPAISNDLAVNDAGVLGGGLWPAQTQRMRTYTKVQKRGSVGRSIDVNRYRGYDELRHDLARMFGIEGQLEDPQTSDWKLVYVDHENDILLVGDDPWEEFVNCVQSIKILSSAEVQQMSLDGNFAGVPVTNQACSGGDSGNAWRGHYDDNSATSFNR

SEQ ID NO: 156 - DNA, 63 bp, Arabidopsis thaliana
  source 1..63: mol_type = genomic DNA
tagttgtcctggtcgctgatcatccctttccgacaactctgttgctctttctctcacgtttga

SEQ ID NO: 157 - DNA, 54 bp, Arabidopsis thaliana
  source 1..54: mol_type = genomic DNA
tgatcatccctttccgacaactctgttgctctttctctcacgtttgagtcttaa

SEQ ID NO: 158 - DNA, 99 bp, Arabidopsis thaliana
  source 1..99: mol_type = genomic DNA
tgagtcttaatcttcgattgtccttggtcatccttaagttttgtttttctctttcgattcattttccagatttctttttatagaaacccgtctctttga

SEQ ID NO: 159 - DNA, 171 bp, Arabidopsis thaliana
  source 1..171: mol_type = genomic DNA
taagttttgtttttctctttcgattcattttccagatttctttttatagaaacccgtctctttgatttgattccccccaagaaaggaaagcaaaacaaagaagagcagctgaatcaagtaactttgtgggtgtttttgaattatctgactcgagataaagagcttcgttaa

SEQ ID NO: 160 - DNA, 96 bp, Arabidopsis thaliana
  source 1..96: mol_type = genomic DNA
tgatttgattccccccaagaaaggaaagcaaaacaaagaagagcagctgaatcaagtaactttgtgggtgtttttgaattatctgactcgagataa

SEQ ID NO: 161 - DNA, 156 bp, Arabidopsis thaliana
  source 1..156: mol_type = genomic DNA
tgactcgagataaagagcttcgttaaaaacggaatcttttttaaggtcgtatgctttgtttgtctctccggagattccgcgtgtgaaaagtcgtggtttttgatctctttgagatacttaaaatttccgagaaattggcggctctgtcggagataa

SEQ ID NO: 162 - DNA, 93 bp, Arabidopsis thaliana
  source 1..93: mol_type = genomic DNA
taaagagcttcgttaaaaacggaatcttttttaaggtcgtatgctttgtttgtctctccggagattccgcgtgtgaaaagtcgtggtttttga

SEQ ID NO: 163 - DNA, 81 bp, Arabidopsis thaliana
  source 1..81: mol_type = genomic DNA
tgaaaagtcgtggtttttgatctctttgagatacttaaaatttccgagaaattggcggctctgtcggagataaaaagctga

SEQ ID NO: 164 - DNA, 102 bp, Arabidopsis thaliana
  source 1..102: mol_type = genomic DNA
taaaatttccgagaaattggcggctctgtcggagataaaaagctgagatcttggagttccccatttctgattaacgattctctctgctgttacttgagctag

SEQ ID NO: 165 - DNA, 78 bp, Arabidopsis thaliana
  source 1..78: mol_type = genomic DNA
taggaagctattaacaacttgagtgaaagtttagggcttttgatgaagctctcacgcaaagagcttagctttgtataa

SEQ ID NO: 166 - DNA, 81 bp, Arabidopsis thaliana
  source 1..81: mol_type = genomic DNA
tgaagctctcacgcaaagagcttagctttgtataagctcagattcagattatttattgggtttattcttcagagaaagtaa

SEQ ID NO: 167 - DNA, 69 bp, Arabidopsis thaliana
  source 1..69: mol_type = genomic DNA
tagctttgtataagctcagattcagattatttattgggtttattcttcagagaaagtaaagttgagtga

SEQ ID NO: 168 - DNA, 96 bp, Arabidopsis thaliana
  source 1..96: mol_type = genomic DNA
taagctcagattcagattatttattgggtttattcttcagagaaagtaaagttgagtgatcatgaaagctccttcatcaaatggagtttctcctaa

SEQ ID NO: 169 - protein, 811 aa, Arabidopsis thaliana
MKLSTSGLGQQGHEGEKCLNSELWHACAGPLVSLPSSGSRVVYFPQGHSEQVAATTNKEVDGHIPNYPSLPPQLICQLHNVTMHADVETDEVYAQMTLQPLTPEEQKETFVPIELGIPSKQPSNYFCKTLTASDTSTHGGFSVPRRAAEKVFPPLDYTLQPPAQELIARDLHDVEWKFRHIFRGQPKRHLLTTGWSVFVSAKRLVAGDSVIFIRNEKNQLFLGIRHATRPQTIVPSSVLSSDSMHIGLLAAAAHASATNSCFTVFFHPRASQSEFVIQLSKYIKAVFHTRISVGMRFRMLFETEESSVRRYMGTITGISDLDSVRWPNSHWRSVKVGWDESTAGERQPRVSLWEIEPLTTFPMYPSLFPLRLKRPWHAGTSSLPDGRGDLGSGLTWLRGGGGEQQGLLPLNYPSVGLFPWMQQRLDLSQMGTDNNQQYQAMLAAGLQNIGGGDPLRQQFVQLQEPHHQYLQQSASHNSDLMLQQQQQQQASRHLMHAQTQIMSENLPQQNMRQEVSNQPAGQQQQLQQPDQNAYLNAFKMQNGHLQQWQQQSEMPSPSFMKSDFTDSSNKFATTASPASGDGNLLNFSITGQSVLPEQLTTEGWSPKASNTFSEPLSLPQAYPGKSLALEPGNPQNPSLFGVDPDSGLFLPSTVPRFASSSGDAEASPMSLTDSGFQNSLYSCMQDTTHELLHGAGQINSSNQTKNFVKVYKSGSVGRSLDISRFSSYHELREELGKMFAIEGLLEDPLRSGWQLVFVDKENDILLLGDDPWESFVNNVWYIKILSPEDVHQMGDHGEGSGGLFPQNPTHL

SEQ ID NO: 170 - DNA, 105 bp, Arabidopsis thaliana
  source 1..105: mol_type = genomic DNA
taaagagttatagctactgtagtagctggaagtgaagaagattttttaatagattgtacggaaaaattagggttttcaaagtttggtttcttgaagttgaattag

SEQ ID NO: 171 - protein, 788 aa, Arabidopsis thaliana
MEFDLNTEIAEVEEEENDDVGVGVGGGTRIDKGRLGISPSSSSSCSSGSSSSSSSTGSASSIYSELWHACAGPLTCLPKKGNVVVYFPQGHLEQDAMVSYSSPLEIPKFDLNPQIVCRVVNVQLLANKDTDEVYTQVTLLPLQEFSMLNGEGKEVKELGGEEERNGSSSVKRTPHMFCKTLTASDTSTHGGFSVPRRAAEDCFAPLDYKQQRPSQELIAKDLHGVEWKFRHIYRGQPRRHLLTTGWSIFVSQKNLVSGDAVLFLRDEGGELRLGIRRAARPRNGLPDSIIEKNSCSNILSLVANAVSTKSMFHVFYSPRATHAEFVIPYEKYITSIRSPVCIGTRFRMRFEMDDSPERRCAGVVTGVCDLDPYRWPNSKWRCLLVRWDESFVSDHQERVSPWEIDPSVSLPHLSIQSSPRPKRPWAGLLDTTPPGNPITKRGGFLDFEESVRPSKVLQGQENIGSASPSQGFDVMNRRILDFAMQSHANPVLVSSRVKDRFGEFVDATGVNPACSGVMDLDRFPRVLQGQEICSLKSFPQFAGFSPAAAPNPFAYQANKSSYYPLALHGIRSTHVPYQNPYNAGNQSSGPPSRAINFGEETRKFDAQNEGGLPNNVTADLPFKIDMMGKQKGSELNMNASSGCKLFGFSLPVETPASKPQSSSKRICTKVHKQGSQVGRAIDLSRLNGYDDLLMELERLFNMEGLLRDPEKGWRILYTDSENDMMVVGDDPWHDFCNVVWKIHLYTKEEVENANDDNKSCLEQAALMMEASKSSSVSQPDSSPTITRV

SEQ ID NO: 172 - DNA, 84 bp, Arabidopsis thaliana
  source 1..84: mol_type = genomic DNA
taggtgtgggttctgttctgtcttggctttgtctcccccgcttatctctccctctctttctctctctctctctctttccactaa

SEQ ID NO: 173 - DNA, 225 bp, Arabidopsis thaliana
  source 1..225: mol_type = genomic DNA
taaattgttttttttttgctctttaaaaagtatatttttttccaatctgcaaattttagtctatgtctgttccttgtgctccctcttctcagtacctgcaaatggaggaagaagaatccttctctgaaacccttgttctcatttgattcctccttcctctctcttcttctctctctgtctctgattcgttattccacacttatgactcatctttcccgtcaatag

SEQ ID NO: 174 - DNA, 90 bp, Arabidopsis thaliana
  source 1..90: mol_type = genomic DNA
tagtctatgtctgttccttgtgctccctcttctcagtacctgcaaatggaggaagaagaatccttctctgaaacccttgttctcatttga

SEQ ID NO: 175 - DNA, 60 bp, Arabidopsis thaliana
  source 1..60: mol_type = genomic DNA
tgaaacccttgttctcatttgattcctccttcctctctcttcttctctctctgtctctga

SEQ ID NO: 176 - DNA, 105 bp, Arabidopsis thaliana
  source 1..105: mol_type = genomic DNA
tgattcctccttcctctctcttcttctctctctgtctctgattcgttattccacacttatgactcatctttcccgtcaatagctaagtttgcctcttctttgtga

SEQ ID NO: 177 - DNA, 105 bp, Arabidopsis thaliana
  source 1..105: mol_type = genomic DNA
tgaaatttagctgaaaaaggagaggaattccgaattctgtcacttcaaagctcgaattttgcaaactttcctttgatgggttttacttgttttgttgtaatctga

SEQ ID NO: 178 - DNA, 69 bp, Arabidopsis thaliana
  source 1..69: mol_type = genomic DNA
tagctgaaaaaggagaggaattccgaattctgtcacttcaaagctcgaattttgcaaactttcctttga

SEQ ID NO: 179 - DNA, 174 bp, Arabidopsis thaliana
  source 1..174: mol_type = genomic DNA
tgaaaaaggagaggaattccgaattctgtcacttcaaagctcgaattttgcaaactttcctttgatgggttttacttgttttgttgtaatctgattaaaaatagaaactttttgttttcttcttgtctccttttgctcttaaaagagaagctttttcaatggaatttgacttga

SEQ ID NO: 180 - DNA, 51 bp, Arabidopsis thaliana
  source 1..51: mol_type = genomic DNA
tgattaaaaatagaaactttttgttttcttcttgtctccttttgctcttaa

SEQ ID NO: 181 - protein, 731 aa, Oryza sativa
MASSASSSSSPSSRPPLMALPSFYRPPWPSERGGEQRATDCWAGSPAAGGGRARATAMGIDLNNTASGGEEDAPAPGPVCRDLWHACAGPVVSLPRRGSAVVYLPQGHLSAAGAGGGIRGEVAVALPPHVACRVVDVELCADAATDEVYARLALRAEGEVFERNLHGGGIEREDDMEDGDEERKSRMLHMFCKTLTASDTSTHGGFSVPRRAAEDCFPPLDHKQLRPSQELVAKDLHGAKWRFRHIYRGQPRRHLLTTGWSSFVNKKKLVSGDAVLFLRGDDGELRLGVRRATQLKNEAIFKAFSSESSKMRTLSAVADSLKHGSVFHICYNPRATASEYVVPYWKFVKSFNHPVCIGMRFKFHFESEDVNERRSGMIAGVSEVDPIRWPGSKWRSLLVRWEDATDCNSQNRVSPWEIEIVGGSISVAHSLSASSSKRTKLCPQGNLDVPALYGNGRPDSVETEKFPRVLQGQELMGSRTHRATCSPQSIDITKSKSFDAWRFLTDTRSCMLGSSTSRLPVQYSGYTHQSVSFGESIGFPEVLQGQEISQTVPPFQGMLPDACSAKSRYELKNYVCTPATMNGLSSANEGYCLSLSTVPPSPPSSLMLYQTGVPQLELASKNNDKSGNDSQPALRQHKLLSETSWDQFKIGKASTPGNATKPGNGGREVDRTSCRLFGFSLTEKIIPTDKDGEKEVSYETDCQNPRMLDLFGYNCSTPGALHALCAAPLGI

SEQ ID NO: 182 - DNA, 180 bp, Oryza sativa
  source 1..180: mol_type = genomic DNA
tgactcgctccagccagcagcagcagcagcagccactgctcgcgacggaacagacgacaccaccacgacgcccgcgtcataaaggcatgggcattgtcgcaggctagccacagcgacggggtagcagcagcatcagcagcagcagcggcaacagcgaccggcattgatagcggcagctga

SEQ ID NO: 183 - DNA, 63 bp, Oryza sativa
  source 1..63: mol_type = genomic DNA
tagccacagcgacggggtagcagcagcatcagcagcagcagcggcaacagcgaccggcattga

SEQ ID NO: 184 - DNA, 78 bp, Oryza sativa
  source 1..78: mol_type = genomic DNA
tagcggcagctgaggaaggaagagggcgcgcggtcgccgcaggagcggagcagggggggcatgggctgggcgggctga

SEQ ID NO: 185 - DNA, 75 bp, Oryza sativa
  source 1..75: mol_type = genomic DNA
tgaggaaggaagagggcgcgcggtcgccgcaggagcggagcagggggggcatgggctgggcgggctgagcgctga

SEQ ID NO: 186 - protein, 622 aa, Oryza sativa
PEHGGGGSGGGGGGGGADAATDEVYARLALVAEGEMLQRNFREGGGEDGAGEMEGCDAEKKPRMPHMFCKTLTASDTSTHGGFSVPRRAAEDCFPPLDYKTVRPSQELIAVDLHGTQWKFRHIYRGQPRRHLLTIGWSSFVNRKKLVSGDAVLFLRGDDGQLRLGVRRAVQLRNEALFEPVNSSDSKLRILSSVASSLENKSVFHICFNPRSGASEFIVPYWRLLKSLNHPFSIGMRFRVCYESEDANERSAGLISGISEVDPIRWPGSRWKCLLVRWDDSTDSSHQNRVSPWEIERVGGSVSVTHSLSSGSKRTKLHFPQGSLDTPFLNGNGHPDSMGTENFHRVLQGQEFRGSRSHGVVCSESPGVPNFQSPDNRRFSADMRGYMMPASGPPQRNTEFTYQPIGFSESLGFPEVLQGQEMSQVVPLFRGATFGARTQNDRVVSANSVHRSAAQSGLLASTLGHPISQFTLSSSKVSSPSSVLMFNQATAPNHETVSGTNNKGMHVSQFASQEMLSETVTWPGTQRQTPSEITSNQFALARIPAPPSGAESGLPKRDAGRSSCRLFGFSLTGNMLGEDGEGLDDGAIEAGCENPPVLELFGHSHSTPGALHALCAAAPLGM

SEQ ID NO: 187 - DNA, 99 bp, Oryza sativa
  source 1..99: mol_type = genomic DNA
taacgcgctcgtcgtcggcgtcgtcggctgccctccaccgcaatcaaggccaatttggcgcgcggcgaggaggaggagggaggaggagatggggattga

SEQ ID NO: 188 - protein, 712 aa, Oryza sativa
MTGIDLNTVEEDEEEAAEEVAANGSSPAPARAGAVCLELWHACAGPVAPLPRKGGVVVYLPQGHLEHLGDAPAAAAAAAAVPPHVFCRVVDVTLLADAATDEVYAQLSLVPEKEEVARRADDGEGEDGDGMKQRFARMPHMFCKTLTASDTSTHGGFSVPRRAAEDCFPPLDYSQQRPSQELVAKDLHSTEWRFRHIYRGQPRRHLLTTGWSAFVNKKKLVSGDAVLFLRGDDGELRLGVRRAAQLKNGSAFPALYNQCSNLGTLANVAHAVATESVFNIYYNPRLSQSEFIVPYWKFMKSLSQPFSVGLRFKMRYESEDATERRYTGIITGSGDTDPMWHGSKWKCLLVRWDDDAEFRRPNRVSPWEIELTSSVSGSHLSTPHSKRLKPCLPHVNPEYMVPRGGGCPDFAESAQFHKVLQGQELLGFKSHGGTAAATSQPCEARHLQYIDERSCSSDASNSILGVPRLGDRAPLGNPGFSYHCSGFGESHRLQKVLQGQELFRPYRGTLVDASMGSNGFHQQDSPRAPGVVNKWQAQLHGRAAFHGPPALALPSQSSSPPSVLMFQQANSKMPRLEFGHGQLDKHENDRRVRFGPSEGIERREQRIPLQPYPTSGEVIDGQVTVEKSHSPGRHGKDGPDNKAVGTNSCKIFGISLTEKVPAREELDDGDANYSLQSLKQVPKSLGNSCATVHEQRPVVGRVIDISTMDMMI

SEQ ID NO: 189 - DNA, 99 bp, Oryza sativa
  source 1..99: mol_type = genomic DNA
tagcaacctacggacaccacccccacccgcttcatttatcacttgtctccccctctccctcgcctgctccgctgctcttgtgcttcgcgcctcgcctga

SEQ ID NO: 190 - protein, 720 aa, Zea mays
MGIDLNMADGESQERRPPPAVCRELWHACAGPVVALPRRGSLVVYLPQGHLAAAGGGNVAVDLPPHVACRVADVELCVSAAVIATSVCFAGRGCIGLHCFDDEARMAVCTVQADAATDEVYARLALVAEGEAFGRNLRGGGVDGDDDMEDFDVERKSRMLHMFCKTLTASDTSTHGGFSVPRRAAEDCFPPLDYNQLRPSQELVAKDLHGAKWKFRHIYRGQPRRHLLTTGWSSFVNKKKLVSGDAVLFLRGDDGELRLGVRRAIQLKNEALFDDFSCDSTKRHTLLAVADSLKHKSVFHISYNPRATASEYIIPHHKFLKSLNLPFCIGARINLQCHNEDVSERRSGMVVHVSEIDPMKWPGSKWRSLLVRWEDGVECNGQDRVSPWEIEIAGGSVSVAHSVSASSSKRTKLCPQGNLDVPTMWNGNGCTDSVETGKFPRVLQGQDLMSFRTRHVPSAPQTVEAAKLQSSGASRFLSNARGCALGGPTSRLAVHNSDFTYQSVGFNESIGFSEVLQGQEISRAVPMFQGMMSEACSLKGGYGLHSYMRTPVAVTGLSATTQECSLTLSTPPGAQVPSVYPDNIFNRTVVRQLGLASKFDGGATNAQQSVPFDRPREIWSKPQHEISDQTKMDHFETRRASAPGDDAAKHGSGGEVVRKTSCRLFGFSLTEKILPADDDGIKEVTYEPECQNPRMLDLFGYNCSAPSAALPALCAAPFGM

SEQ ID NO: 191 - DNA, 285 bp, Zea mays
  source 1..285: mol_type = genomic DNA
taaagcagcagcctgccgcagctgccttgcagcggccgcgcacggtggccagtagcgtgcaacagcagcgcagagcgcagcgccgtgccactccgcaacgacgcagcagtagcagcgccccacaccctggtgcggcggtgccccacggcaaaaggaaacagcccgtccaatccatcccctcaccccattgcctctccctctccctcacccgcacctcaccactcctcggcctcacttcttcacaagttcacatcagcagcagctcacgaaacaggagcgtcataa

SEQ ID NO: 192 - DNA, 60 bp, Zea mays
  source 1..60: mol_type = genomic DNA
tagcgtgcaacagcagcgcagagcgcagcgccgtgccactccgcaacgacgcagcagtag

SEQ ID NO: 193 - DNA, 198 bp, Zea mays
  source 1..198: mol_type = genomic DNA
tagcagcgccccacaccctggtgcggcggtgccccacggcaaaaggaaacagcccgtccaatccatcccctcaccccattgcctctccctctccctcacccgcacctcaccactcctcggcctcacttcttcacaagttcacatcagcagcagctcacgaaacaggagcgtcataaagacatggtcgtcgcaggatag

SEQ ID NO: 194 - DNA, 171 bp, Zea mays
  source 1..171: mol_type = genomic DNA
taaagacatggtcgtcgcaggatagcctcagacccagcgacgcagcggcagcgacccaggaagcaacagcgaccggcatcgatcgcggcagccgaggcggggggacgggaggagggagggagcggtcgccgcaggagcagagcggcacggcatggggtcctgagtgactga

SEQ ID NO: 195 - DNA, 141 bp, Zea mays
  source 1..141: mol_type = genomic DNA
tagcctcagacccagcgacgcagcggcagcgacccaggaagcaacagcgaccggcatcgatcgcggcagccgaggcggggggacgggaggagggagggagcggtcgccgcaggagcagagcggcacggcatggggtcctga

SEQ ID NO: 196 - DNA, 66 bp, Zea mays
  source 1..66: mol_type = genomic DNA
tgactgacgactgagtgtctgactgagaaggagatggctggagcgcgacggccccggagccattaa

SEQ ID NO: 197 - DNA, 117 bp, Zea mays
  source 1..117: mol_type = genomic DNA
tgagaaggagatggctggagcgcgacggccccggagccattaatggggcgctcatcctcctcgtcctcccactcctcgcctcccccccgccaggtcgcggtcggctccgctttttaa

SEQ ID NO: 198 - DNA, 144 bp, Zea mays
  source 1..144: mol_type = genomic DNA
taatggggcgctcatcctcctcgtcctcccactcctcgcctcccccccgccaggtcgcggtcggctccgctttttaaccggcttgtcctggagcggggcgaacggcgaacctcacgggcgccggaacacgaaccggaaccctag

SEQ ID NO: 199 - protein, 622 aa, Zea mays
MAGIDLNTVEEEDEEEAEALPLPGPGGRGAVCLELWHACAGPVPPLPRKGSAVVYLPQGHLEHIGGDAARGAAASAVPPHVLCRVVDVTLHADGATDEVYARVSLLPEDEDAEKRAQAQARVREDEDRRDGEDGGAMRPLARTPHMFCKTLTASDTSTHGGFSVPRRAAEDCFPPLDYSQQRPSQELVAKDLHGTEWKFRHIYRGQPRRHLLTTGWSAFVNRKKLISGDAVLFLRGEDGVLRLGVRRAAQLKIVTPIPAPHNQCSSNSNLGNVAQAVATKTVFHIYYNPRLTQSEFIVPYWKFTRSFNQPISVGMRCRMRYESDDASERRCTGIIIGSREADPIWYGSKWKCLVVRWDDGIECRWPNRVSPWEIELTGSVSGSQMCAPSSKRLKPCLPQVNPEIVLPNGSVSSDFAGSARFHKVLQGQELLGFKTHDGPAISASQATEARNLQYSDERSNNNLGIPRLGVRSPTGIPGFPYHCSGFGESQRFQKVLQGQEVFHPFRGGCLADGHIRTAGMYQPDGEAIEECTGTVNMHSPVSGAEHESNNESTVENGCKIFGISLAEKIRSCDEADSCSAKCNSRLQPLKSQMPKSLGSCWATVHEQRPVVGRVVDVSATDM

SEQ ID NO: 200 - DNA, 165 bp, Zea mays
  source 1..165: mol_type = genomic DNA
tagccccgtataggcctgcctctcgtctgcgccctactccccaccacttccctcccttccccgcctacaagcaagccgccccagccattcatggccatcaccaccaccatgtgtcgccgccactccacctccacctcccacccactcctctcaccgcaccactaa

SEQ ID NO: 201 - protein, 582 aa, Zea mays
MFGRNINDGETEEKNGEEEDGDGEKKHASHMFCKTLTASDTSTHGGFSVPRRAAEDCFPPLDYEQLRPSQELIAKDLHGMKWRFRHIYRGQPRRHLLTTGWSSFINKKKLVSGDAVLFLRGNDGELRLGVRRAVQLKNEALLEAVNCTDSKLLMLSAVANSLDNRSIFHICFNPRVGASEFIVPYCKFLKSLNYHFSVGTRFKVGCENEDANERSFGLIIGISEVDPIHWPGSKWKSLLIKWDGATKYSHQNRVSPWDIEGVGSSVSVTHRLSSSVSKRTKLCFPPSDLDTPILDGNGRPDSVETERFHRVLQGQELVHSSIHGTACSHSSDSPRCQGSYGRRFSADAWNCKMNDVMSGPRHLNATGFAYQPLGFSESVKFSEVLQGQEMSQAVPSFMRSAFNSGTQNGRVRPFDYVQRSDATQGYALQQFNLPATEVHSPSSVLMFNQTMVPHAELDGATNREEVHGSRYLSSNAIGREAEPWPSMQQQRASVNGSEPLDTTEASAPARNAESGSVGRGAGRSNCKLFGFSLTEKILGTDGGGVKEGNYEVDRQTPRVLDLFGHGSTPGALHALCAAPLGI

SEQ ID NO: 202 - DNA, 462 bp, Zea mays
  source 1..462: mol_type = genomic DNA
tagctggtcccgacgcccgagggccgacggcgagagcagaccgggcctcctccctgtgcggaaacagttcagtcctgctcggctcggcacaccaccaacggttacgcgcctacagacgccgtgccgcggcgcccgaggacaaggaaccccaccaaaaccacccgccgaaaccgagccaaaagctctgtggtcccctcgtccacggccaccgaccccaccatccataaactcgccacggccactgccgcagcggtccggtcaggaggggcgggaacaggaaccgcggcccgcacgcatcgaagccgctgcggtctgcgctctgcggtggtgggctctggctctgcctctggctctggcgcaggggcgtgggggacgggactggcattgccactgcccagccgcgggagcggcccctccggggggacaagcgcattaatgccactgccgcctccgccgatttaa

SEQ ID NO: 203 - DNA, 339 bp, Zea mays
  source 1..339: mol_type = genomic DNA
taaactcgccacggccactgccgcagcggtccggtcaggaggggcgggaacaggaaccgcggcccgcacgcatcgaagccgctgcggtctgcgctctgcggtggtgggctctggctctgcctctggctctggcgcaggggcgtgggggacgggactggcattgccactgcccagccgcgggagcggcccctccggggggacaagcgcattaatgccactgccgcctccgccgatttaactccccgcctctatcgccctcaccttcacctgtacgccgtcccgcttcgcttcggagaggtcgctgcctgtgcctgccatcaacgttccctcgctggttga

SEQ ID NO: 204 - protein, 659 aa, Zea mays
MAGIDLNDTVEEDEEEAEPGNACSQQSRTSSAATFPPPPPNQPRPSAAVCLELWHACAGPVAPLPRKGSVVVYLPQGHIEHLGDAAAAGGGAPPPVALPPHVFCRVVDVTLHADASTDEVYAQLALVAENEDVARRLRGRSEDGSAEDGDEGETVKQRFSRMPHMFCKTLTASDTSTHGGFSVPRRAAEDCFPPLDYSQQRPSQELVAKDLHGTEWRFRHIYRGQPRRHLLTTGWSAFVNKKKLVSGDAVLFLRGDNGELRLGVRRAAQLKNGSAFPALYNQCLNLGSLPNVAHAVATKSVFHIYYNPRLSQSEFIIPFSKFIKSFSQPFSAGSRFKVKYESDDASERRCTGIIAGIGDADPMWRGSKWKCLMVRWDDDVDFRQPNRISPWEIELTSSVSGSHMSAPNAKRLKPCLPHVNPDYLVPNGSGRPDFAESAQFHKVLQGQEVFHPYRGTLVDASLSNSGFHQQDGSHVPTQASKWHAQLHGCAFRGQQAPAVPSQSSSPPSVLMFQRGDPKMSPFEFGHFHVNKKEDRRAMFVHAGGIGGTEQTTMLQAHHVSGGTGNRDVTVEKSHPAVAAASDNREVSKNSCKIFGISLTEKVPAMKEKGCGDINTNYPSPFLSLKQQVPKSLGNSCATVHEQRPVVARVIDVSTVDMMI

SEQ ID NO: 205 - DNA, 141 bp, Zea mays
  source 1..141: mol_type = genomic DNA
tgaccagtgaccgaccacccccacaccgctccggcgccccaatcctctccctcttctttttcactactactactgtactgcacggtcgccaagcgccagaacgcagtggagaacggggggcaggactccaacaagcgttga

SEQ ID NO: 206 - DNA, 345 bp, Zea mays
  source 1..345: mol_type = genomic DNA
tgaccgaccacccccacaccgctccggcgccccaatcctctccctcttctttttcactactactactgtactgcacggtcgccaagcgccagaacgcagtggagaacggggggcaggactccaacaagcgttgatttctaccggcacgggcacgggcacgggcacgggcgtccaccctcacgcaccctgcgtcttttccggctgccgctgctggctggctggctctggctcacagctacaggctacagtgaccgccacgcaacccacactgtctctgtcctcgtctccctctcccctcctagctctagctggataggtgggctctggggaggaggaggagggtag

SEQ ID NO: 207 - DNA, 120 bp, Zea mays
  source 1..120: mol_type = genomic DNA
tgatttctaccggcacgggcacgggcacgggcacgggcgtccaccctcacgcaccctgcgtcttttccggctgccgctgctggctggctggctctggctcacagctacaggctacagtga

SEQ ID NO: 208 - DNA, 54 bp, Zea mays
  source 1..54: mol_type = genomic DNA
tgaccgccacgcaacccacactgtctctgtcctcgtctccctctcccctcctag

SEQ ID NO: 209 - DNA, 99 bp, Zea mays
  source 1..99: mol_type = genomic DNA
taggtagtagctgcctataggcctcggcccccattcatggccattaccacgatgtgtcaccccaccacaccgccctctccgatgctgcctccctcatga

SEQ ID NO: 210 - DNA, 162 bp, Zea mays
  source 1..162: mol_type = genomic DNA
taggcctcggcccccattcatggccattaccacgatgtgtcaccccaccacaccgccctctccgatgctgcctccctcatgataaccctctccctggtggttgttctttgccttgttgccgtgcagcctccacccccaccctcctcattaatcacttgctag

SEQ ID NO: 211 - DNA, 69 bp, Zea mays
  source 1..69: mol_type = genomic DNA
taaccctctccctggtggttgttctttgccttgttgccgtgcagcctccacccccaccctcctcattaa

SEQ ID NO: 212 - protein, 571 aa, Zea mays
MEDFDAERKSGMLHMFCKTLTASDTSTHGGFSVPRRAAEDCFPPLDYNQLRPSQELVAKDLHGAKWKFRHIYRGQPRRHLLTTGWSSFVNKKKLVSGDAVLFLRGDDGELRLGVRRAIQLKNEALFEDFNSDSTKRHSLTAVADSLKNRSVFHISYNPRATASEYIIPYAKFLKSLNHPVCIGARINFQCHNEDVSERRSGVVVRISEIDPMKWPGSKWRSLLVRWEDGAECNGQDRVSPWEIEIAGGSVSVSHSLSASSSKRTKLCPQGNLDVPAMWNGCTDSMETGKLPRVLQGQELMSFRTCHVPCAPQTAEAAKLQSSDASRFLSNAHGCALGGPTSRLAVHSSGFTYQCVGFNESIGFSEVLQGQEISRAVPKFQGMMSEACSLKDRYGLHSYMRAPVAVNGLSATTQECSLALSTPPGAQVPSLYPGNVVNRTVVPQLGLARKFGGGGTNGQQSGPFDRPRELWTRPQHETPDQMNLDQFETRRPSVPVDAAKLGSGGGEVRKTSCRLFGFSLTEKILPADDDDVKEVSYETECQNPRMLDLFGYNCSTPSAALPALCAAAPFGM

SEQ ID NO: 213 - DNA, 129 bp, Zea mays
  source 1..129: mol_type = genomic DNA
taatacgtgtgcaactacaactacaagcgctacgcaaaatgcagcagctactcccgtgaaaaaggtagcaggaccgatcaaacgaaccgagacgaatttcaggtcaaaaagaccacgtccggatgctag

SEQ ID NO: 214 - DNA, 78 bp, Zea mays
  source 1..78: mol_type = genomic DNA
tagcaggaccgatcaaacgaaccgagacgaatttcaggtcaaaaagaccacgtccggatgctagattacaacacctga

SEQ ID NO: 215 - DNA, 138 bp, Zea mays
  source 1..138: mol_type = genomic DNA
tgaccaccgatgcgcttagcggagtcatgcatgctggctcctgctgggggaacagtggcacctaaagcagcagcctgccgcgcacggtgtccagtaagcgcgcaacagcagcagcagccagcagcgggggcctcctaa

SEQ ID NO: 216 - DNA, 81 bp, Zea mays
  source 1..81: mol_type = genomic DNA
tagcggagtcatgcatgctggctcctgctgggggaacagtggcacctaaagcagcagcctgccgcgcacggtgtccagtaa

SEQ ID NO: 217 - DNA, 138 bp, Zea mays
  source 1..138: mol_type = genomic DNA
taaagcagcagcctgccgcgcacggtgtccagtaagcgcgcaacagcagcagcagccagcagcgggggcctcctaaaaaatacccggcgcggcgccctagggcaaaaggaaacagctcgtccagtccgtagtccatag

SEQ ID NO: 218 - DNA, 99 bp, Zea mays
  source 1..99: mol_type = genomic DNA
taagcgcgcaacagcagcagcagccagcagcgggggcctcctaaaaaatacccggcgcggcgccctagggcaaaaggaaacagctcgtccagtccgtag

SEQ ID NO: 219 - DNA, 138 bp, Zea mays
  source 1..138: mol_type = genomic DNA
tagggcaaaaggaaacagctcgtccagtccgtagtccatagtactccatttgcaaattgcattgccacacctcggctcatccccacctcacctcggcctcacttcacaagttcacatcacgaaacaggagcgtcataa

SEQ ID NO: 220 - DNA, 129 bp, Zea mays
  source 1..129: mol_type = genomic DNA
tagtccatagtactccatttgcaaattgcattgccacacctcggctcatccccacctcacctcggcctcacttcacaagttcacatcacgaaacaggagcgtcataaagacatgggcgtcgcagggtag

SEQ ID NO: 221 - DNA, 144 bp, Zea mays
  source 1..144: mol_type = genomic DNA
tagtactccatttgcaaattgcattgccacacctcggctcatccccacctcacctcggcctcacttcacaagttcacatcacgaaacaggagcgtcataaagacatgggcgtcgcagggtagcctcagacggtcagaccagtga

SEQ ID NO: 222 - DNA, 81 bp, Zea mays
  source 1..81: mol_type = genomic DNA
taaagacatgggcgtcgcagggtagcctcagacggtcagaccagtgacgcagcggcagcggcagcgggcatggggtcctga

SEQ ID NO: 223 - DNA, 63 bp, Zea mays
  source 1..63: mol_type = genomic DNA
tagcctcagacggtcagaccagtgacgcagcggcagcggcagcgggcatggggtcctgagtga

SEQ ID NO: 224 - DNA, 198 bp, Zea mays
  source 1..198: mol_type = genomic DNA
tgacgcagcggcagcggcagcgggcatggggtcctgagtgaccgactgcgaaggagatggctggagcgcgacggccccggagccattaatggggtgctcatcctcctccccctcctcgcctcctccccgccgggtcgcggacgcggtcggctccgccttttaaccggcctgtccgggagcggggcgccggaaccctag

SEQ ID NO: 225 - DNA, 129 bp, Zea mays
  source 1..129: mol_type = genomic DNA
tgagtgaccgactgcgaaggagatggctggagcgcgacggccccggagccattaatggggtgctcatcctcctccccctcctcgcctcctccccgccgggtcgcggacgcggtcggctccgccttttaa

SEQ ID NO: 226 - DNA, 51 bp, Zea mays
  source 1..51: mol_type = genomic DNA
tgaccgactgcgaaggagatggctggagcgcgacggccccggagccattaa

SEQ ID NO: 227 - DNA, 126 bp, Zea mays
  source 1..126: mol_type = genomic DNA
taaccggcctgtccgggagcggggcgccggaaccctagtgctgctggtggcacgcggcgcgccccatgggcatcgatctcaacatggtggacggcgagggccacgagcgccgcccgccgccggtga

SEQ ID NO: 228 - DNA, 345 bp, Zea mays
  source 1..345: mol_type = genomic DNA
tagtgctgctggtggcacgcggcgcgccccatgggcatcgatctcaacatggtggacggcgagggccacgagcgccgcccgccgccggtgacgtgccgggagctgtggcacgcgtgcgcggggcccgtggtcgcgctaccgcgccggggcagcctggtcgtgtacctgccgcagggccacctggccgcggcgggcggcggggacgtcgctgcggacctgccgccgcacgtggtgtgccgcgtcgcggatgtcgagctatgcgcggatgcggcgacggacgaggtgtgcgcgcggctggcgctggttgcggagggcgaggtaaaaatatcctgttcaaaaaattga

SEQ ID NO: 229 - DNA, 234 bp, Zea mays
  source 1..234: mol_type = genomic DNA
tgacgtgccgggagctgtggcacgcgtgcgcggggcccgtggtcgcgctaccgcgccggggcagcctggtcgtgtacctgccgcagggccacctggccgcggcgggcggcggggacgtcgctgcggacctgccgccgcacgtggtgtgccgcgtcgcggatgtcgagctatgcgcggatgcggcgacggacgaggtgtgcgcgcggctggcgctggttgcggagggcgaggtaa

SEQ ID NO: 230 - DNA, 129 bp, Zea mays
  source 1..129: mol_type = genomic DNA
taaaaatatcctgttcaaaaaattgaaccgattggatttgcatgctcacagggatcgcggagattcatgttccccgtgtttggaggagatatgtgtgccagccctgtttccttttctgttaagacttaa

SEQ ID NO: 231 - DNA, 99 bp, Zea mays
  source 1..99: mol_type = genomic DNA
tgaaccgattggatttgcatgctcacagggatcgcggagattcatgttccccgtgtttggaggagatatgtgtgccagccctgtttccttttctgttaa

SEQ ID NO: 232 - DNA, 69 bp, Zea mays
  source 1..69: mol_type = genomic DNA
taagacttaagactgctaccactaccagtgtgctgcattcgggaggaatctgggcggtggtggagttga

SEQ ID NO: 233 - protein, 707 aa, Triticum aestivum
MGIDLNSVEEDGEAAAAAAVGVCGELWHACAGPGVALPRRGSAVVYLPQAHLAAGGGEAPAPAPAPAGAPRVPPHVACRVVGVELCADAATDEVYARLALVADAEMFRQNVRESASEEGEDGMAGGDGEKKPRMPHMFCKTLTASDTSTHGGFSVPRRAAEDCFAPLDYKQVRPSQELVAKDLHGTQWRFRHIYRGQPRRHLLTTGWSSFVNKKKLVSGDAVLFLRGDDGELRLGVRRAVQLRNEALFKAVNSNESKLHTLSAVASSLENRSIFHVCFDPRSGASEFIVPYWRFSKSLNHSFSIGMRFKDSNESDDANERSTGLISGISEVDPIRWPGSKWRCLQVRWDDGNHCNHQRRVSPWEIERVGGSISVTDCLSASSSKRAKLYFPQGNLDAPVTDGNGYPDSVETGSFHRVLQGQELLMGSRAHGAARSQSPDVAKFRTPDHQRFSANARSYIPQQSPVEFPYHSSGFGESLRFPEVLQGQEMSQALRFYQGTAFDARAQHGGVVGPFGYAQRSAAPGGLSPAAQGYALGQFTPSAAAAAKVSSPSSVLMFNQATVPQFDLEGRASSYRGAYGGQCPPAMEMARETEEAWPCAQRRTPSVTGTGCRRFEEWIKASTPASADGGRARPAAAGDVVGRSNCRLFGFSLTDQKVLGGAGEGDAKEGDADDVVAECADPRVLDLFGRGHPAPGALHAIVAAPLGM

SEQ ID NO: 234 - DNA, 60 bp, Triticum aestivum
  source 1..60: mol_type = genomic DNA
tgagcgagcgagcacagcgaccgcgtccgggggagaccacggtgggggcgagcaccataa

SEQ ID NO: 235 - DNA, 75 bp, Triticum aestivum
  source 1..75: mol_type = genomic DNA
taactcgctcctctgcgccgccatccaccgcgctgcgacggcattaacgcggcgggggttggtctagctagctag

SEQ ID NO: 236 - protein, 735 aa, Triticum aestivum
MAGIDLNTVEEDEEESSAEVLLARSGGGGDCSSQSHSHSHSHSHSQTHSGHGQGSSSVAAATPPRPSAVCLELWHACAGPVAPMPRKGGVVVYLPQGHLDHLGDAPAPSPAAVPPHVFCRVVDVTLHADASTDEVYAQLSLLPENEEVVRRMREATEDGSGGEDGETVKQRFARMPHMFCKTLTASDTSTHGGFSVPRRAAEDCFPPLDYSQQRPCQELVAKDLHGTEWKFRHIYRGQPRRHLLTTGWSAFVNKKKLVSGDAVLFLRGDDGELRLGVRRAVQLKNGSAFPALYSQCSNLGTLANVAHAVATKSMFQIFYNPRLSQSEFIVPYWKFTKSFSQPFSVGSRFKMRYESEDAAERRYTGIITGTGDADPMWRGSKWKCLLVRWDDDGEFRRPNRVSPWEIELTSSASGSHLAAPTSKRMKPYLPHANPEFTVPHGGGRPDFAESAQVRKVLQGQELLGYRTHDGTAVATSQPCEARNLQYIDERGCSNNGSNNVLGGVPSHGVKTPLGIPYHCSGFGESQRFQKVLQGQEVFRPYRGSLVDARMRSGGFHQQDGPYASALLDKWRTQQQHAFGFGSSAPVLPSQPSLSPPSVLMFQQADPKVSRFEFGQGHLDKNMDDPYARFVSAEAIGRGEQTLSLRPHLGSEVIDTRVAVENKGVAPTNSCKIFGISLAEKVRARDEMGCDDGGANYPSSTQPLKQQVPKSLGNSCATVHEQRPVVGRAIDVQQWI

SEQ ID NO: 237 - DNA, 51 bp, Triticum aestivum
  source 1..51: mol_type = genomic DNA
tagccacacagcctctccccgaacccactcccgctccggtcgcttcattag

SEQ ID NO: 238 - protein, 653 aa, Triticum aestivum
MGIDLNAAGEPGKAAAVSVCRDLWHACAGPVVALPRRGSAVVYLPQAHLAGAGGGGDVPVALPPHVACRVVDVELCADPATDEVYARLAMVAEGEVFEKNMGGGRFEGEDDMEDGDGERKSRMLHMFCKTLTASDTSTHGGFSVPRRAAEDCFPPLDYQQVRPSQELVAKDLHGAKWRFRHIYRGQPRRHLLTTGWSSFVNKKKLVSGDAVLFLRGDDGELRLGVRRAIQLKNEALLKSFNSNSSKIHTLSAMANSLKHRSVFHICYNPRAAASEFIVPYWKFLKSLNRPFCIGMRFKIQYGSEDVNERRSGIITGINEVDPIRWTGSKWKSLQVRWEDGTDCNSQNRLSPWDIEIVGGSVSIAQSLSASSSKRTKLCPQGNVDVPTLYGNGRPDSVGADKLPRVLQGQELMGSGTHRVTCPPRPGGATEFRRSDGTGFLADTRSCMLSGPASRLPPQSPYFAYQPVGFGESVGFPEVLQGQEVCQTVPLFQGMVSDTCSAKGGYGLYNYMCTSSAMNGGFSSAAQGYPLSLSTPPPAEVASPSSTVAAPQLWLASKNNEEAAIGSQPNPLGTRKASGDGAAKLGSDGRKVARTSCMLFGFSLTEKMLPTEEDCAKEGNHEVDCQNPRMLDLFGYNRATPGALHALCAAPLGI

SEQ ID NO: 239 - DNA, 300 bp, Triticum aestivum
  source 1..300: mol_type = genomic DNA
taaaatcgtgggcgactcgtgtcccagtccccagcaggctcaggctcaggctcagccacagcccacagcgacgcggcagcggcagcggcagtggcagcaggaacagcaccccaacagcgaccttgcttccgcgcgcggcagccgagggaggggggaggaggagatcgagggcggcatggggacggggggctagaacaaggagatggctgcagagcgacgccctcgggggcattaatggggccctccccctgctccgcatcccgctgctcccacatcctccccgcggccgcgcgcggttga

SEQ ID NO: 240 - DNA, 87 bp, Triticum aestivum
  source 1..87: mol_type = genomic DNA
taatggggccctccccctgctccgcatcccgctgctcccacatcctccccgcggccgcgcgcggttgattcgcccgcctgcttttaa